(19) United States
(12) Patent Application Publication
(10) Pub. No.: US 2013/0122085 A1
(43) Pub. Date: May 16, 2013

(54) PHARMACEUTICAL COMPOSITIONS OF SELECTIVE ANDROGEN RECEPTOR MODULATORS AND METHODS OF USE THEREOF

(71) Applicant: GTx, Inc., Memphis, TN (US)

(72) Inventors: James T. DALTON, Lakeland, TN (US); Matthew GOSNELL, Lakeland, TN (US)

(73) Assignee: GTx, Inc., Memphis, TN (US)

(21) Appl. No.: 13/666,741

(22) Filed: Nov. 1, 2012

Related U.S. Application Data
(60) Provisional application No. 61/555,179, filed on Nov. 3, 2011.

(51) Int. Cl. A61K 31/277

(52) U.S. Cl. CPC A61K 31/277
USPC 424/451; 514/522

(57) ABSTRACT

This invention provides a pharmaceutical composition comprising Compound I-V, including inter alia solid dosage forms of powder-filled capsule formulations, liquid-filled softgel capsules (softgels), tablets, and sustained release dosage forms, and uses thereof in treating a variety of diseases or conditions in a subject, for example, treating a muscle wasting disease and/or disorder, a bone related disease and/or disorder, metabolic syndrome, diabetes and associated diseases, and others.

### CROSS-REFERENCE TO RELATED APPLICATIONS

This Application claims priority from U.S. Provisional Application Ser. No. 61/555,179, filed Nov. 3, 2011; which is hereby incorporated by reference in its entirety.

### FIELD OF INVENTION

This invention provides pharmaceutical compositions comprising a SARM compound, including inter alia solid dosage forms of powder-filled capsule formulations, liquid-filled softgel capsules (softgels), tablets, and sustained release dosage forms, and uses thereof in treating a variety of diseases or conditions in a subject, including, inter alia, muscle wasting diseases and/or disorders, bone related diseases and/or disorders, metabolic syndrome, diabetes and associated diseases, and others.

### BACKGROUND OF THE INVENTION

The androgen receptor (“AR”) is a ligand-activated transcriptional regulatory protein that mediates induction of male sexual development and functions through its activity with endogenous androgens. Androgens are generally known as the male sex hormones. The androgenic hormones are steroids which are produced in the body by the testes and the cortex of the adrenal gland or can be synthesized in the laboratory. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Testosterone is the principal steroid secreted by the testes and is the primary circulating androgen found in the plasma of males. Testosterone is converted to DHT by the enzyme 5 alpha-reductase in many peripheral tissues. DHT is thus thought to serve as the intracellular mediator for most androgen actions (Zhou, et al., Molec. Endocrinol. 9:208-18 (1995)). Other steroidal androgens include esters of testosterone, such as the cypionate, propionate, phenylpropionate, cyclopentylpropionate, isocarporate, enanthate, and decanoate esters, and other synthetic androgens such as 7-methyl-nortestosterone (“MENT”) and its acetate ester (Sundaram et al., “7 alpha-Methyl-nortestosterone (MENT): The optimal androgen for male contraception,” Ann Med., 25:199-205 (1993) (“Sundaram”). Because the AR is involved in male sexual development and function, the AR is a likely target for effecting male contraception or other forms of hormone replacement therapy.

Selective androgen receptor modulator (SARM) compounds and a pharmaceutical composition comprising the same are useful for, inter alia a) treating cachexia; b) treating osteoporosis and other bone-related diseases or disorders; c) treating sarcopenia, muscle wasting and other muscle related diseases or disorders; d) lowering circulating lipid levels or improving a lipid profile; e) improving insulin sensitivity; f) treating obesity; g) male contraception; h) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; i) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; j) preventing and/or treating dry eye conditions; k) oral androgen replacement therapy; and/or) decreasing the incidence of, halting or causing a regression of prostate cancer.

The use of Selective Androgen Receptor Modulators (SARMS) for these and other disorders are cited in the literature, for example in: Terry R. Brown., Nonsteroidal selective androgen receptor modulators (SARMs): Designer androgens with flexible structures provide clinical promise, Endocrinology (2004) 145(14): 5417-5419; Yin, D., et al., Key structural features of nonsteroidal ligands for binding and activation of the androgen receptor. Mol. Pharmacol. (2003) 63: 211-223; Yin D., et al. Pharmacodynamics of selective androgen receptor modulators. J. Pharmacol. Exp. Ther. (2003) 304: 1334-1340; Yin, D., et al. Pharmacology, pharmacokinetics, and metabolism of acetothiolutamide, a novel nonsteroidal agonist for the androgen receptor. J. Pharmacol. Exp. Ther. (2003) 304(8): 1323-1333; Chen, J. et al. In vitro and in vivo structure-activity relationships of novel androgen receptor ligands with multiple substituents in the B-ring, Endocrinology (2005) 146(12): 5444-5454.

Selective Androgen Receptor Modulators and their synthesis are also described in U.S. Pat. Nos. 6,995,284; 6,071,957; 6,482,861; 7,041,844; 7,129,377; 6,492,554; 6,569,896; 7,026,500; 6,998,500; 6,838,484; 7,645,898; 7,705,182; 7,772,433; 7,968,721; 7,977,386; and 7,968,603 which are incorporated herein in their entirety by reference.

### SUMMARY OF THE INVENTION

In one embodiment, this invention provides a pharmaceutical composition comprising a softgel capsule comprising the S-isomer of Compound I:

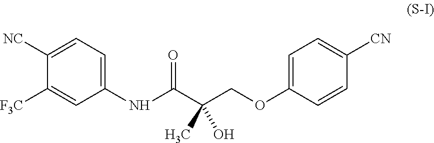 [SMILES: C[C@](O)(COc1ccc(C#N)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or a combination thereof. In another embodiment, the softgel capsule consists essentially of 3 mg, 1 mg, or 0.5 mg of Compound S-I.

In one embodiment, this invention provides a pharmaceutical composition comprising a micronized S-isomer of Compound I:

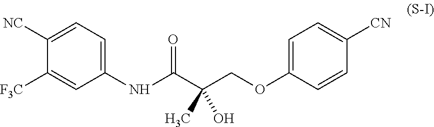 [SMILES: C[C@](O)(COc1ccc(C#N)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or a combination thereof. In another embodiment, the composition is in a solid form. In another embodiment, the composition is in the form of a capsule. In another embodiment, the capsule comprises 0.5 mg, 1 mg or 3 mg of Compound S-I. In another embodiment, the composition is in the form of a tablet. In another embodiment, the tablet comprises 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.

In one embodiment, this invention provides a sustained release pharmaceutical composition comprising an S-isomer of Compound I:

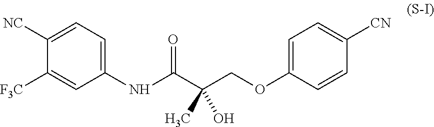 [SMILES: C[C@](O)(COc1ccc(C#N)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or a combination thereof. In another embodiment, the sustained release pharmaceutical composition is administered topically, transdermally, orally or via an infusion. In another embodiment, the composition comprises 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the composition comprises a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.

### BRIEF DESCRIPTION OF THE FIGURES

The present invention will be understood and appreciated more fully from the following detailed description taken in conjunction with the appended figures in which:

FIG. 1: schematically depicts a flow diagram illustrating an embodiment of a powder-filled capsule manufacturing process of the SARM of Compound I (S-I).

FIG. 2: schematically depicts embodiments of different blenders used in a process for the preparation of a tablet and/or capsule of this invention. A) Shear blender B) KG-5 blender; C) 4 qt V-Shell blender.

FIG. 3: graphically depicts distal trabecular bone density as a function of ovariectomy, S-I, and/or alendronate treatment.

FIG. 4: schematically depicts a flow diagram illustrating manufacturing process of 3 mg softgel capsules of Compound (S-I).

FIG. 5: Certificates of Analysis for: (A) S-I 3.0 mg softgels (liquid-filled); and (B) S-I capsules (powder-filled), 3 mg.

FIG. 6: depicts pharmacokinetics data from phase I clinical study: (A) 3 mg S-I Capsule (powder-filled), fasted; (B) 3 mg S-I softgels (liquid-filled), fasted; (C) 3 mg S-I softgel (liquid-filled), fed.

FIG. 7: depicts measurements of changes in fasting serum HDL (Δ HDL) and changes in lean body mass (Δ LBM) of OVX female rats that were treated for 14 days with (1) 3 mg/kg/day DHT administered daily via subcutaneous injection, (2) 0.13 mg/kg/day S-I administered daily via subcutaneous injection, and (3) 0.13 mg/kg/day S-I administered at a constant rate via subcutaneous infusion by an Alzet pump.

### DETAILED DESCRIPTION OF THE INVENTION

In one embodiment, this invention provides a pharmaceutical composition comprising an S-isomer of selective androgen receptor modulator (SARM) Compound I:

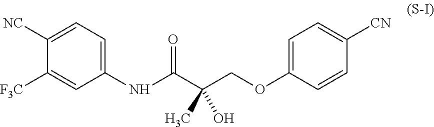 [SMILES: C[C@](O)(COc1ccc(C#N)cc1)C(=O)Nc1ccc(C#N)c(C(F)(F)F)c1]

or its isomer, polymorph pharmaceutically acceptable salt, N-oxide, hydrate or any combination thereof, and a carrier, diluent, or any combination thereof.

In one embodiment, this invention provides a pharmaceutical composition comprising an R-isomer of selective androgen receptor modulator (SARM) Compound I:

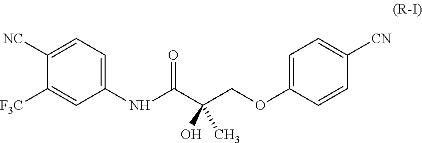 [SMILES: C[C@@](O)(COc1ccc(C#N)cc1)C(=O)Nc1ccc(C#N)c(C(F)(F)F)c1]

or its isomer, polymorph, pharmaceutically acceptable salt, N-oxide, hydrate or any combination thereof and a carrier, diluent, or any combination thereof.

In one embodiment, this invention provides a pharmaceutical composition comprising a racemic mixture of selective androgen receptor modulator (SARM) Compound I:

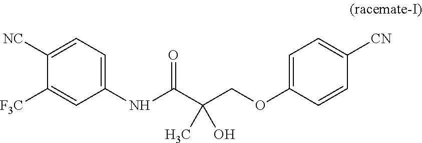 [SMILES: CC(O)(COc1ccc(C#N)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof. In another embodiment, the pharmaceutical composition comprises Compound I in a mixture of the (R) and (S) isomers.

In one embodiment, this invention provides a pharmaceutical composition comprising an S-isomer of a selective androgen receptor modulator (SARM) Compound II:

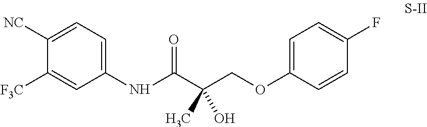 [SMILES: C[C@](O)(COc1ccc(F)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising an R-isomer of a selective androgen receptor modulator (SARM) Compound II:

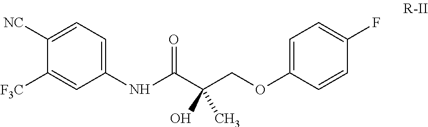 [SMILES: C[C@@](O)(COc1ccc(F)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising a racemic mixture of selective androgen receptor modulator (SARM) Compound II:

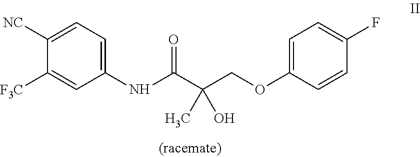 [SMILES: CC(O)(COc1ccc(F)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof. In another embodiment, the pharmaceutical composition comprises Compound II in a mixture of the (R) and (S) isomers.

In one embodiment, this invention provides a pharmaceutical composition comprising an S-isomer of the selective androgen receptor modulator (SARM) Compound III:

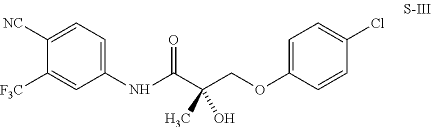 [SMILES: C[C@](O)(COc1ccc(Cl)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising an R-isomer of the selective androgen receptor modulator (SARM) Compound III:

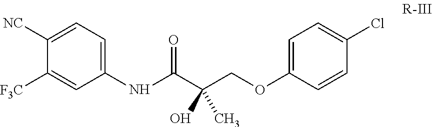 [SMILES: C[C@@](O)(COc1ccc(Cl)cc1)C(=O)Nc1ccc(C#N)c(C(F)(F)F)c1]

or its isomer, polymorph, pharmaceutically acceptable salt, N-oxide, hydrate or any combination thereof,and a carrier, diluent, or any combination thereof.

In one embodiment, this invention provides a pharmaceutical composition comprising a racemic mixture of selective androgen receptor modulator (SARM) Compound III:

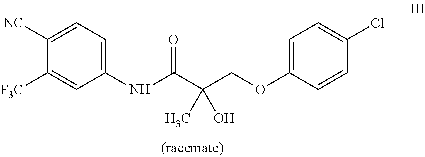 [SMILES: CC(O)(COc1ccc(Cl)cc1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof. In another embodiment, the pharmaceutical composition comprises Compound III in a mixture of the (R) and (S) isomers.

In one embodiment, this invention provides a pharmaceutical composition comprising an S-isomer of the selective androgen receptor modulator (SARM) Compound IV:

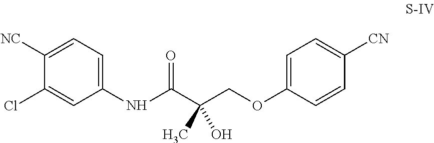 [SMILES: C[C@](O)(COc1ccc(C#N)cc1)C(=O)Nc1ccc(C#N)c(Cl)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising an R-isomer of the selective androgen receptor modulator (SARM) Compound IV:

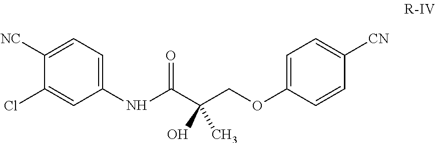 [SMILES: C[C@@](O)(COc1ccc(C#N)cc1)C(=O)Nc1ccc(C#N)c(Cl)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising a racemic mixture of selective androgen receptor modulator (SARM) Compound IV:

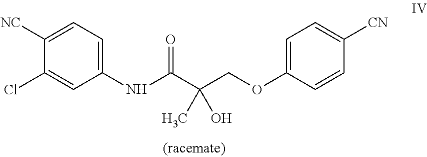 [SMILES: CC(O)(COc1ccc(C#N)cc1)C(=O)Nc1ccc(C#N)c(Cl)c1]

or its isomer, polymorph, pharmaceutically acceptable salt, N-oxide, hydrate or any combination thereof, and a carrier, diluent, or any combination thereof. In another embodiment, the pharmaceutical composition comprises Compound IV in a mixture of the (R) and (S) isomers.

In one embodiment, this invention provides a pharmaceutical composition comprising an S-isomer of the selective androgen receptor modulator (SARM) Compound V:

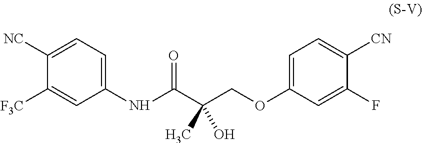 [SMILES: C[C@](O)(COc1ccc(C#N)c(F)c1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising an R-isomer of the selective androgen receptor modulator (SARM) Compound V:

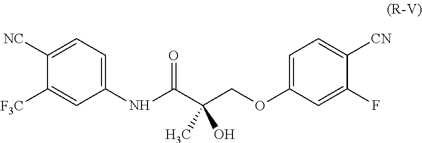 [SMILES: C[C@@](O)(COc1ccc(C#N)c(F)c1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof.

In one embodiment, this invention provides a pharmaceutical composition comprising a racemic mixture of selective androgen receptor modulator (SARM) of Compound V:

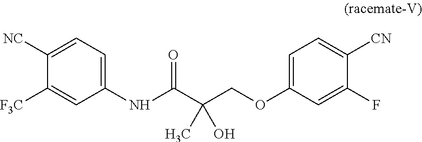 [SMILES: CC(O)(COc1ccc(C#N)c(F)c1)C(=O)Nc1ccc(C#N)c(C(F)(F)F)c1]

or its isomer, polymorph, pharmaceutically acceptable salt, N-oxide, hydrate or any combination thereof, and a carrier, diluent, or any combination thereof. In another embodiment, the pharmaceutical composition comprises Compound V in a mixture of the (R) and (S) isomers.

It is to be understood that reference to a compound as described herein, i.e. Compounds I, II, III, IV and V as described herein, is to be considered to include the S-isomer, R-isomer or racemic mixture thereof.

In one embodiment, this invention provides a pharmaceutical composition comprising an isomer of Compound I, Compound II, Compound III, Compound IV or Compound V (Compound I-V). In another embodiment, this invention provides a pharmaceutical composition comprising a polymorph of Compound I-V. In another embodiment, the polymorph is an amorphous form of Compound I-V. In another embodiment, this invention provides a pharmaceutical composition comprising a pharmaceutically acceptable salt of Compound I-V. In another embodiment, this invention provides a pharmaceutical composition comprising a hydrate of Compound I-V. In another embodiment, this invention provides a pharmaceutical composition comprising an N-oxide of Compound I-V. In another embodiment, this invention provides a pharmaceutical composition comprising a combination of any of an isomer, polymorph, pharmaceutically acceptable salt, N-oxide, hydrate or any combination thereof, of Compound I-V.

In one embodiment, this invention provides a pharmaceutical composition comprising a polymorph of Compound I. In another embodiment, the polymorph is an amorphous form of Compound S-I. In another embodiment, this invention provides a pharmaceutical composition comprising a pharmaceutically acceptable salt of Compound S-I. In another embodiment, this invention provides a pharmaceutical composition comprising a hydrate of Compound S-I. In another embodiment, this invention provides a pharmaceutical composition comprising an N-oxide of Compound S-I. In another embodiment, this invention provides a pharmaceutical composition comprising a combination of any of an isomer, polymorph, pharmaceutically acceptable salt, N-oxide, hydrate or any combination thereof, of Compound S-I. In another embodiment, the polymorph is an amorphous form of Compound S-I. In another embodiment, the polymorph of compound S-I is form A as described in Example 2. In another embodiment, the polymorph of compound S-I is form B′ as described in Example 2. In another embodiment, the polymorph of compound S-I is form B″ as described in Example 2. In another embodiment, the polymorph of compound S-I is form D as described in Example 2. In another embodiment, the polymorph of compound S-I is form C as described in Example 2.

In one embodiment, the pharmaceutical composition comprises one SARM compound as an active ingredient. In alternative embodiment, the pharmaceutical composition comprises more than one SARM compound, for example a combination of two, three or more SARM compounds. In some embodiments, the active ingredient in the pharmaceutical composition is an isomer, polymorph, pharmaceutically acceptable salt, N-oxide, hydrate or any combination thereof, of the SARM compound.

In some embodiments, the term “isomer” includes, but is not limited to, optical isomers and analogs, structural isomers and analogs, conformational isomers and analogs, and the like.

In one embodiment, this invention encompasses a pharmaceutical composition and uses thereof comprising various optical isomers of Compounds I-V as herein described. It will be appreciated by those skilled in the art that Compounds I-V of the present invention contain at least one chiral center. Accordingly, Compounds I-V in the pharmaceutical composition and used in the methods of the present invention may exist in, and be isolated in, optically-active or racemic forms. Some compounds may also exhibit polymorphism, as described in U.S. Ser. Nos. 60/960,012; 12/209,137 (now U. S. Pat. No. 7,977,386); Ser. No. 12/228,100 (now U. S. Pat. No. 7,968,603); and U.S. Ser. No. 13/153,427 as incorporated herein by reference. It is to be understood that the pharmaceutical composition(s) of the present invention encompass any racemic, optically-active, polymorphic, or stereroisomeric form, or mixtures thereof, which form possesses properties useful in the methods as described herein. In another embodiment, the pharmaceutical composition comprises Compound I-V in a pure (S)-isomer. In one embodiment, the pharmaceutical composition comprises Compound I-V in a pure (R)-isomer. In another embodiment, the pharmaceutical composition comprises Compound I-V in a mixture of the (R) and (S) isomers. In another embodiment, Compound I-V is a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase). In some embodiments, the synthesis of racemate or (R) or (S) isomers of Compound I-V is as disclosed in U.S. application Ser. Nos. 11/505,363, 11/826,195, U.S. Pat. No. 6,995,284, and U.S. Pat. No. 7,968,721, which are hereby incorporated by reference, in their entirety. In some embodiments, the compounds may be synthesized by methods similar thereto, modified as will be appreciated by one skilled in the art, or in some embodiments, the synthesis may be accomplished by any means known to one skilled in the art. The pharmaceutical composition of the invention includes “pharmaceutically acceptable salts” of the compounds as herein described, which may be produced, by reaction of such compounds with an acid or base.

Suitable pharmaceutically-acceptable salts of amines of Compound I-V may be prepared from an inorganic acid or from an organic acid. In one embodiment, inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrate, persulfates, phosphates, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, organic salts of amines may comprise aliphatic, cycloaliphatic, aromatic, araliphatic, heterocyclic, carboxylic and sulfonic classes of organic acids, including acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxylates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonate gluconates, glutamates, glycolates, glucorates, glucoheptanoates, glycerophosphates, gluceptates, glycollylarsanilates, glutarates, glutamates, heptanoates, hexanoates, hydroxymaleates, hydroxycarboxlic acids, hexylresorcinates, hydroxybenzoates, hydroxynaphthoate, hydrofluorate, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartarates, theophyllineacetates, p-toluenesulfonates(tosylates), trifluoroacetates, terephthalates, tannates, teoclates, trihaloacetates, triethiodide, tricarboxylates, undecanoates and valerates.

In one embodiment, inorganic salts of carboxylic acids or phenols may comprise ammonium; alkali metals including lithium, sodium, potassium, cesium; alkaline earth metals, including calcium, magnesium, aluminium, zinc, barium, choline or quaternary ammonium.

In another embodiment, organic salts of carboxylic acids or phenols may comprise arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In one embodiment, salt forms of SARM compounds may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

The preparation, isolation and purification of these salts are well known to those skilled in the art, as they are commonly employed in a therapeutic setting for a variety of uses other than described herein. Specific preparation procedures for each salt are described in general terms in Remington's Pharmaceutical Sciences, Mack Publishing Company, Easton, Pa., 16th Ed., 1982, which is incorporated herein by reference.

In one embodiment, the invention also includes N-oxides of the amino substituents of the SARM compounds described herein. In some embodiments, esters of the phenolic compounds can be prepared with aliphatic and aromatic carboxylic acids, for example, acetic acid and benzoic acid esters.

In another embodiment, this invention further includes a pharmaceutical composition comprising derivatives of the compounds described herein. The term “derivatives” includes but is not limited to ether derivatives, acid derivatives, amide derivatives, ester derivatives and the like.

The term “hydrate” includes but is not limited to hemihydrate, monohydrate, dihydrate, trihydrate and the like.

In another embodiment, the invention provides a pharmaceutical composition comprising metabolites of the described compounds. The term “metabolite” refers, in some embodiments, to any substance produced from another by metabolism or a metabolic process. In one embodiment, the metabolite of Compound I-V is a glucuronide metabolite. In another embodiment, the metabolite of Compound I-V is hydroxylation metabolite. In another embodiment, the metabolite of Compound I-V is an amide hydrolysis metabolite. In another embodiment, the metabolite of Compound I-V is a methylated metabolite. In another embodiment, the metabolite of Compound I-V is a sulfated metabolite.

The pharmaceutical composition of this invention may comprise crystals of Compound I-V. In some embodiments, the pharmaceutical composition may comprise polymorphs of Compound I-V. The term “crystal” refers to a substance in a crystalline state. The term “polymorph” refers, in some embodiments, to a particular crystalline state of a substance, having particular physical properties described by X-ray diffraction patterns, IR spectra, phase transition point, and the like. In another embodiment, this invention is directed to pharmaceutical composition consisting essentially of polymorph A of Compound S-I as described in Example 2. In another embodiment, this invention is directed to pharmaceutical composition consisting essentially of polymorph B′ of Compound S-I as described in Example 2. In another embodiment, this invention is directed to pharmaceutical composition consisting essentially of polymorph B″ of Compound S-I as described in Example 2. In another embodiment, this invention is directed to pharmaceutical composition consisting essentially of polymorph C of Compound S-I as described in Example 2. In another embodiment, this invention is directed to pharmaceutical composition consisting essentially of polymorph D of Compound S-I as described in Example 2. In another embodiment, this invention is directed to pharmaceutical composition consisting essentially of polymorph A, B′, B″, C , D or any combination thereof of Compound S-I as described in Example 2.

In some embodiments, this invention provides methods of use, which comprise administering a pharmaceutical composition comprising the described compounds. In some embodiments, the term “pharmaceutical composition” refers to a composition administered to a subject comprising a “therapeutically effective amount” of the active ingredient, i.e. Compound I-V, and/or another SARM, and optionally with other therapeutic compounds disclosed herein, together with a pharmaceutically acceptable carrier or diluent. A “therapeutically effective amount” refers, in some embodiments, to that amount which provides a therapeutic or prophylactic effect for a given condition and administration regimen.

In one embodiment, this invention includes a variety of oral dosage forms such as powder-filled capsules (0.1 mg, 0.3 mg, 1 mg, 3 mg, and 10 mg), compressed tablets (0.1 mg, 0.3 mg, 1 mg, and 3 mg), and liquid-filled softgels (0.5 mg, 1 mg, 3 mg, 4 mg, 5 mg and 9 mg) of S-I. As mentioned in Example 2, S-I is present in multiple polymorphic solid forms which could impact a variety of physical properties of a pharmaceutical composition which delivers S-I as a solid. Polymorphs can present challenges for formulating an active pharmaceutical ingredient (API) into an oral dosage form. For instance, if polymorphs have different physical properties such as solubility, dissolution rate, melting point, etc. then poorly controlled relative levels of individual polymorphs or interconversion between polymorphs in the pharmaceutical composition can cause variation in the physical or chemical stability or the pharmacokinetics of the pharmaceutical composition. Also once multiple polymorphic forms have been observed, it is always possible that further polymorphs exist with unknown physicochemical properties. The liquid-filled softgel pharmaceutical composition of this invention removes the uncertainties of working with multiple polymorphs because the API is in a solution phase, but still allows for a convenient and attractive solid oral dosage form.

In some embodiments, any of the pharmaceutical compositions of this invention comprises Compound I-V, in any form or embodiment as described herein. In some embodiments, the pharmaceutical compositions of this invention will consist of Compound I-V, in any form or embodiment as described herein. In some embodiments the pharmaceutical composition of this invention will consist essentially of Compound I-V, in any form or embodiment as described herein. In some embodiments, the term “comprise” refers to the inclusion of the indicated active agent, such as Compound I-V as well as inclusion of other active agents, and pharmaceutically acceptable carriers, excipients, emollients, stabilizers, etc., as are known in the pharmaceutical industry; or the term “consisting essentially of” refers to a pharmaceutical composition, whose only active ingredient is the indicated active ingredient, however, other compounds may be included which are for stabilizing, preserving, etc. the formulation, but are not involved directly in the therapeutic effect of the indicated active ingredient; or the term “consisting essentially of” refers to a pharmaceutical composition, whose only active ingredient from a particular class of compounds is the indicated active ingredient, however, other therapeutic compounds with other activity, for example acting on a molecule downstream in a particular pathway than that of the indicated active, or acting on another pathway, or ameliorating symptoms associated with a particular condition being treated, etc., may be included in the pharmaceutical composition. In some embodiments, the active ingredient is Compound I-V, however other SARM compounds are incorporated therein, and such a pharmaceutical composition may be said to consist essentially of Compound I-V. Any compound as herein described in any combination may be combined with Compound I-V and be referred to herein as a pharmaceutical composition “consisting essentially of Compound I-V. In some embodiments, the term “consisting essentially of” may refer to components which facilitate the release of the active ingredient. In some embodiments, the term “consisting” refers to a pharmaceutical composition, which contains the active ingredient and a pharmaceutically acceptable carrier or excipient.

As used herein, the term “administering” refers to bringing a subject in contact with a SARM compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

As used herein, the term “subject” refers to a living individual who takes or receives one or more compounds of the instant invention. In one embodiment, a subject may be a mammal, a canine, a feline, a primate or belong to another class of living organisms. In another embodiment, a subject may be a human or an animal.

In some embodiments, the term “about” when in regard to an amount, will refer to an amount that is equal to the indicated amount, or in some embodiments, exceeds or is less than the indicated amount by 1%, or in some embodiments, 2.5%, or in some embodiments, 5%, or in some embodiments, 7.5%, or in some embodiments, 10%, or in some embodiments, 15%, or in some embodiments, any amount therebetween.

In one embodiment, the pharmaceutical composition is administered orally, and is thus formulated in a form suitable for oral administration, i.e. as a solid or a liquid preparation. Suitable solid oral formulations include tablets, dragees, troche, beads, capsules, powder-filled capsules, liquid-filled capsules, liquid-filled softgel capsules (softgels), pills, granules, pellets or powders. Suitable liquid oral formulations include solutions, suspensions, dispersions, emulsions, oils or syrups.

In one embodiment, the pharmaceutical compositions provided herein are sustained or controlled release compositions, i.e. compositions in which the compound of this invention is released over a period of time after administration. Controlled or sustained release compositions include formulation in lipophilic depots (e.g. fatty acids, waxes, oils). Further, in another embodiment, the pharmaceutical compositions are administered by subcutaneous implantation of a pellet, rods or powders. In a further embodiment, the pellet provides for controlled release of a compound as herein described over a period of time. In another embodiment, the composition is an immediate release composition, i.e. a composition in which all of the compound is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:607 (1980); Saudek et al., N. Engl. J. Med. 321:674 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 116-138 (1984). Other controlled release systems are discussed in the review by Langer (Science 249:1627-1633 (1990)). In another embodiment, the pharmaceutical composition is delivered in a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound of the invention. In another embodiment, the pharmaceutical composition is delivered in a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In one embodiment, through an intravenous infusion. In another embodiment, through an implantable osmotic pump. In another embodiment, through a transdermal patch. In another embodiment, through a liposome.

In one embodiment, compositions according to this invention may be in the form of controlled-release or sustained-release compositions, wherein the compounds of this invention and the optional carrier are encapsulated or otherwise contained within a material such that they are released following oral administration or via the skin in a controlled manner over time. The compound of this invention and optional carrier may be contained within matrixes, liposomes, vesicles, microcapsules, microspheres and the like, or within a solid particulate material, all of which are selected and/or constructed to provide release of the compound of this invention over time. The compound of this invention and the optional carrier may be encapsulated together (e.g., in the same capsule) or separately (in separate capsules).

As used herein, the term “controlled release” refers to the solid composition in the form of a tablet, capsule, softgel capsule, etc., that releases the compound of this invention over an extended period of time as opposed to a rapid release, and where the extended period of time can be measured in seconds, minutes, hours, days, or weeks. In another embodiment, the composition is delivered in a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound of the invention. In another embodiment, the composition is delivered in a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.

As used herein, the term “delayed release” refers to the solid composition in the form of a tablet, capsule, softgel capsule, etc., that releases the compound of this invention after a period of time as opposed to a immediate release, and where the period of time can be initiated upon the pharmaceutical composition entering particular portion of the gastrointestinal tract.

In one embodiment, delayed release pharmaceutical compositions contain a drug core, capsule or tablet, and/or a delayed release coating surrounding the core (capsule or tablet). The core is a mixture of the active pharmaceutical compound, a diluent, a binding agent and optionally other conventional processing aids such as anti-sticking agents, fillers and glidants. The delayed release coating surrounds the core and is comprised of an enteric polymer and optionally other conventional processing aids such as surfactants, plasticizers and anti-sticking agents.

In a preferred embodiment, the present invention is a delayed release pharmaceutical dosage formulation for oral administration comprising a core with a compound of this invention, and an enteric polymer surrounding the core.

In one embodiment this invention is directed to a delayed release formulation comprising a compound of formula I-V or its isomer, polymorph, pharmaceutically acceptable salt, N-oxide, hydrate or any combination thereof, and a carrier, diluent, or any combination thereof.

In one embodiment this invention is directed to a controlled release formulation comprising a compound of formula I-V or its isomer, polymorph, pharmaceutically acceptable salt, N-oxide, hydrate or any combination thereof, and a carrier, diluent, or any combination thereof.

Although, as discussed above, multiple polymorphs can be a challenge in formulating an API, their existence can also present opportunities for optimizing drug delivery. For instance, a known untoward effect of agonizing the AR is the lowering of HDL. Although not proven to have deleterious cardiovascular implications (e.g., testosterone-replacement therapies are not necessarily associated with increased cardiovascular risk), the ability to limit HDL-lowering with a SARM by judicious selection of a mode of drug delivery would be advantageous. Example 11 demonstrates that a controlled release system (subcutaneous infusion via osmotic pump) increased efficacy (greater increase in lean body mass (LBM)) while limiting the untoward HDL-lowering effect. Consequently it is believed that controlled release dosage forms such as patches, topically applied formulations (ointments, creams, oils, etc.), sub-dermal implants, implanted pumps (osmotic or mechanized), controlled release injections, controlled release oral dosage forms (i.e. slowly dissolving matrices, extrusion-limited systems, etc.), delayed release oral dosage forms (i.e. pH sensitive coatings), etc., may exhibit advantageous pharmacodynamics (PD). These PD advantages can be rationalized by lowering SARM levels in the liver thereby eliminating the first-pass as the drug is absorbed in the gut before delivery to the systemic circulation via the enterohepatic vasculature. Alternatively, changing the pharmacokinetics of delivery of the hormone to the target tissues (muscle, liver, etc.) may yield a favorable PD profile. Regardless of the mechanism behind the phenomenon seen in Example 12 for S-I, the use of polymorphs of S-I disclosed herein, including amorphous forms or polymorphs, in development of HDL-sparing delivery systems for S-I and other SARMs is explicitly contemplated by this invention.

In one embodiment of the present invention, the SARM compounds are formulated in a capsule. In accordance with this embodiment, the pharmaceutical composition of the present invention comprise in addition to the SARM active compound an inert carrier or diluent.

In one embodiment of the present invention, the SARM compounds are formulated in a softgel capsule. In accordance with this embodiment, the pharmaceutical composition of the present invention comprise in addition to the SARM active compound an inert carrier or diluent.

In one embodiment, the capsule can be a gelatin capsule or a polysaccharide capsule such as a cellulose capsule. The gelatin capsule can be formulated as soft gelatin capsule (softgel) or as hard gelatin capsule. Any gelatin known by one of skill in the art to be suitable for preparation of capsules can be used to form the gelatin capsules, including, but not limited to, bovine gelatin, porcine gelatin, fish gelatin, and pure isinglass. In a cellulose capsule the film-forming material can be a cellulosic polymer, including, but not limited to, hydroxypropyl cellulose, hydroxyethyl cellulose, hydroxypropyl methyl cellulose, hydroxymethyl cellulose, methyl cellulose, ethyl cellulose, cellulose acetate, cellulose acetate phthalate, cellulose acetate trimelliate, hydroxypropylmethyl cellulose phthalate, hydroxypropylmethyl cellulose succinate, carboxymethyl cellulose sodium, and mixtures thereof. The capsule can also be formed from pullulan or other glucans such as scieroglucan, polyvinyl alcohol, pectin, modified starches, alginates including sodium, ammonium, potassium, or calcium alginate, or propylene alginate, polyvinyl pyrrolidone, carboxyvinyl polymer, polyacrylic acid, soligel, chitin, chitosan, levan, elsinan, gelatin, collagen, zein, gluten, soy protein isolate, whey protein isolate, casein, or gums including xanthan gum, tragacanth gum, guar gum, acacia gum, Arabic gum, locust bean gum, and gum ghatti. The modified starches can, in particular, be starch ethers or oxidized starch and more particularly hydroxypropylated starch or hydroxyethylated starch. The capsule can take any suitable form known in the pharmacological arts. For example, the capsule can be a hard-shell capsule or a soft-shell capsule. In one particular aspect, the capsule can comprise pullulan. In one form, a capsule can be enterically coated. The capsule can also include stabilizing agents.

In one embodiment, the term “film forming material” refers to a film covering as protection against oxidation, moisture, light, abrasion, rough handling, etc. of solid pharmaceutical materials, tablets, granules and seeds. A pigment is usually present as added protection against light and as a color aid for product identification. In another embodiment, the film is free of roughness, irregularities, cracks or mottled colorations. Film smoothness is important as an aid in swallowing. A hard, shiny surface is desirable for an attractive appearance. Of course, films and coating compositions for ingestion must be edible or physiologically compatible. In another embodiment, film-forming compositions for coating pharmaceutical tablets preferably contain as the film-forming element, a film-forming resinous material, either naturally occurring or synthetic. Normally, film-forming compositions are applied as a liquid coating formulation comprising a liquid carrier medium having dispersed or dissolved therein the film-forming components. In another embodiment, application of the liquid coating formulation is effected by spraying dry pharmaceutical forms in rotation in a coating pan or in a fluidized air bed. After evaporation of the liquid medium, the film coated pharmaceuticals are recovered.

In one embodiment, this invention provides any one of a pharmaceutical composition comprising a selective androgen receptor modulator (SARM) Compound I-V: having mean particle size of between about 0.5-200 μm, or isomer, pharmaceutically acceptable salt, N-oxide, hydrate or any combination thereof, and a carrier, diluent, or any combination thereof. In one embodiment, the pharmaceutical composition of this invention comprises non-micronized Compound I-V, wherein the term “non-micronized” used herein refers to particles having Gaussian type particle size distribution, having a mean particle diameter of about 100 to about 200 microns. In another embodiment, non-micronized particles refer to particles having Gaussian type particle size distribution, having a mean particle diameter of between 200-300 microns. In another embodiment, particles having Gaussian type particle size distribution, have a mean particle diameter of between about 100 to about 150 microns. In another embodiments, to particles having Gaussian type particle size distribution, having a mean particle diameter of between about 100 to about 300 microns.

In one embodiment, the pharmaceutical composition of this invention comprises micronized Compound I-V, wherein the term “micronized” used herein refers to particles having Gaussian type particle size distribution, having a mean particle diameter of less than 100 microns, or in another embodiment, Gaussian type particle size distribution, having a mean particle diameter of less than 100 microns, or in another embodiment, Gaussian type particle size distribution, having a mean particle diameter of less than 60 microns, or in another embodiment, Gaussian type particle size distribution, having a mean particle diameter of less than 36 microns, or in another embodiment, Gaussian type particle size distribution, having a mean particle diameter of less than 16 microns, or in another embodiment, Gaussian type particle size distribution, having a mean particle diameter of less than 10 microns, or in another embodiment, Gaussian type particle size distribution, having a mean particle diameter of less than 6 microns, or in another embodiment Gaussian type particle size distribution, having a mean particle diameter of between 0.5-1 microns, or in another embodiment Gaussian type particle size distribution, having a mean particle diameter of between 5-10 microns, or in another embodiment, Gaussian type particle size distribution, having a mean particle diameter of between 40-60 microns, or in another embodiment, Gaussian type particle size distribution, having a mean particle diameter of between 0.5-30 microns, or in another embodiment, Gaussian type particle size distribution, having a mean particle diameter of between 0.5-200 microns.

In one embodiment, with reference to particles having Gausian type particle size distribution as herein described, such particle distribution will have a standard deviation of between 0.2-0.5 microns. In another embodiment, such particle distribution will have a standard deviation of between 0.3-0.7 microns. In another embodiment, such particle distribution will have a standard deviation of between 0.3-1 microns. In another embodiment, such particle distribution will have a standard deviation of between 0.5-0.7 microns.

In some embodiments, pharmaceutical compositions of this invention comprising micronized Compound I, Compound II, Compound III, Compound IV or Compound V exhibit greater bioavailability. In some embodiments, pharmaceutical compositions of this invention comprising micronized Compound I, Compound II, Compound III, Compound IV or Compound V may be administered at a lower dose than similar compositions comprising larger particle sizes of the compound, yet yielding the same or a comparable therapeutic effect, which in some embodiments may result in reduced side-effects associated with drug administration. In some embodiments, pharmaceutical compositions of this invention comprising micronized Compound I, Compound II, Compound III, Compound IV or Compound V have a longer circulating half life in a subject. In some embodiments, pharmaceutical compositions of this invention comprising micronized Compound I, Compound II, Compound III, Compound IV or Compound V have a longer shelf life than similar compositions comprising larger particle sizes of the compound, which in some embodiments, is a function of greater stability of the compound/composition.

In another embodiment, micronization aids in processing for blend uniformity and time to uniformity. It can further make processing more reproducible and limits the need for crystallization particle size control during the drug synthesis. In another embodiment, micronization speeds dissolution and make absorption quicker due to the dissolution rate. In another embodiment, micronization reduces the risk of aggregation and particle size growth in vivo resulting in reduced/delayed absorption. In another embodiment, uniform micronized particle size promotes more consistent clinical efficacy across various batch productions, by creating more consistant dissolution patterns.

The process used to reduce size of particles is chosen according to the powder characteristics (structure, hardness, and chemical stability), and the chemical size required. The granulation of powder can be defined as the average particle size measured using an appropriate device such as: a particle size counter, or electronic or laser diffraction for smaller granules capable to screen down to a few tens of micrometer. Conventional techniques to reduce particle size may be used, for example grinding in an air-jet mill or impact mill, a ball mill, vibration mill, mortar mill or pin mill In one embodiment, a Jet-O-Mill Micronizer is used.

In one embodiment, this invention provides oral dosage forms of crystalline forms, amorphous forms or hydrate forms of Compound I-V, which can be administered to a mammal (including humans). The dosage form may comprise Compound I-V and, a carrier or diluent. The dosage form, in some embodiments, is in the form of tablets, capsules, softgels, beads, or granulates. The core tablets, beads or granulates may be coated to provide improved swallowability, moisture and light protection, gastric pH-resistance, masking of bitterness, better appearance or any other desired characteristic, as known to those skilled in the art. In another embodiment, this invention provides oral dosage forms of crystalline forms, amorphous forms or hydrate forms of Compound S-I.

In one embodiment, this invention provides a tablet comprising Compound S-I, for example, as described herein in Example 5, which presents physical properties of tablets containing Compound S-I. As exemplified herein, such tablets have been characterized, including their mean hardness, percent of loss (friability), disintegration and mean weight of the tablet.

In one embodiment, this invention provides a capsule comprising compound S-I. In another embodiment, the capsule is a softgel capsule. In another embodiment, this invention provides a softgel capsule comprising Compound S-I, for example, as described herein in Example 7. In another embodiment, this invention provides a softgel capsule consisting essentially of Compound S-I. In another embodiment, this invention provides a softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg dosage of Compound S-I. In another embodiment, this invention provides a softgel capsule consisting essentially of 0.1 mg dosage of Compound S-I. In another embodiment, this invention provides a softgel capsule consisting essentially of 0.3 mg dosage of Compound S-I. In another embodiment, this invention provides a softgel capsule consisting essentially of 0.5 mg dosage of Compound S-I. In another embodiment, this invention provides a softgel capsule consisting essentially of 1 mg dosage of Compound S-I. In another embodiment, this invention provides a softgel capsule consisting essentially of 1.5 mg dosage of Compound S-I. In another embodiment, this invention provides a softgel capsule consisting essentially of 2 mg dosage of Compound S-I. In another embodiment, this invention provides a softgel capsule consisting essentially of 2.5 mg dosage of Compound S-I. In another embodiment, this invention provides a softgel capsule consisting essentially of 3 mg dosage of Compound S-I. In another embodiment, this invention provides a softgel capsule consisting essentially of 4 mg dosage of Compound S-I. In another embodiment, this invention provides a softgel capsule consisting essentially of 5 mg dosage of Compound S-I. In another embodiment, this invention provides a softgel capsule consisting essentially of 9 mg dosage of Compound S-I. In another embodiment, this invention provides a softgel capsule consisting essentially of 10 mg dosage of Compound S-I. In another embodiment, this invention provides a softgel capsule consisting essentially of 30 mg dosage of Compound S-I.

In one embodiment “softgel” capsule refers to a gelatin based shell surrounding a fill material. softgel shells are a combination of gelatin, water, opacifier and a plasticiser such as glycerin and/or sorbitol(s). In one embodiment, gelatin can be replaced with any capsule shell polymer, including inter-alia polymers based on starch and carrageenan. In one embodiment, the softgel shell comprises capsule shell polymer, e.g. gelatin; plasticizer, e.g. sorbitol; and colorant or opacifier, e.g. titanium dioxide. In another embodiment, the sorbitol is Sorbitol Special—Glycerin Blend A810. In one embodiment, the fill material comprises the active agent and a solubilizer or filler. In another embodiment, the active agent comprises compound I-V or its isomer, pharmaceutical acceptable salt, hydrate, N-oxide or any combination thereof. In another embodiment, the active agent comprises compound S-I. In another embodiment, the solubilizer or filler comprises polyethylene glycol including inter alia polyethylene glycol 400.

Softgel capsules are normally produced in a process known as encapsulation using the “Rotary Die Encapsulation” process. In one embodiment a process for preparing softgel capsules was as described in U.S. Pat. No. 7,213,511, U.S. Pat. No. 5,735,105 and U.S. Pat. No. 6,769,226 patents which are incorporated herein by reference.

In one embodiment, “Rotary Die Encapsulation” process refers to a “form/fill/seal process”. Accordingly, two flat ribbons of shell material are manufactured on the machine and brought together on a twin set of rotating dies. The dies contain recesses in the desired size and shape, which cut out the ribbons into a two dimensional shape, and form a seal around the outside. At the same time a pump delivers a precise dose of fill material through a nozzle incorporated into a filling wedge whose tip sits between the two ribbons in between two die pockets at the point of cut out. The wedge is heated to facilitate the sealing process. The wedge injection causes the two flat ribbons to expand into the die pockets, giving rise to the three dimensional finished product. After encapsulation, the softgels are dried for two days to two weeks depending on the product.

In one embodiment, this invention provides pharmaceutical composition that can contain, in addition to the compounds of this invention, excipients such as polyethylene glycol, gelatin, starch, sorbitol, lactose, microcrystalline cellulose, talc, sodium lauryl sulfate, silicic acid, silicon dioxide, aluminum hydroxide, titanium dioxide, calcium silicates, magnesium stearate, and polyamide powder, or mixtures of these substances.

In one embodiment, the term “pharmaceutically acceptable carrier or excipient” refers to a non-toxic, inert solid, diluent, encapsulating material or formulation auxiliary of any type. Excipients in some embodiments comprise diluents, disintegrants, carriers, binders, flow regulators, plastisizers, lubricants, colorants, fillers, solubilizers and solvents. Some examples of materials, which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents such as titanium dioxide, releasing agents, coating agents, sweetening, plasticizers such as sorbitol-glycerin blend, flavoring and perfuming agents, preservatives and antioxidants can also be present in the pharmaceutical composition, according to the judgment of the formulator. Some excipients can serve multiple functions, for example as both binder and disintegrant.

A variety of materials may be used as fillers or diluents. Examples are spray-dried monohydrate or anhydrous lactose, sucrose, dextrose, mannitol, sorbitol, starch (e.g. corn starch, pregeletanized starch), a gum, an acrylate (e.g. polymethylacrylate), magnesium oxide, talc, cellulose (e.g. microcrystalline cellulose), dehydrated, or anhydrous dibasic calcium phosphate calcium carbonate, calcium sulfate, polyethylene glycol, or mixtures thereof. In one embodiment, the filler is polyethylene glycol 400. In another embodiment, the filler is pregelatinized starch. In another embodiment, the filler is lactose monohydrate. In another embodiment, the filler is microcrystalline cellulose.

In one embodiment, pharmaceutical composition of this invention may further comprise binders such as acacia, cornstarch, starch, gelatin, carbomer, cellulose derivatives (such as methylcellulose, ethyl cellulose, carboxymethylcellulose, hydroxypropylmethylcellulose (HPMC), hydroxypropylcellulose, hydroxyethylcellulose, hydroxymethylcellulose), guar gum, povidone, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, bentonites or any combination thereof.

In another embodiment, other excipients such as glidants and coloring agents may also be added to the pharmaceutical composition of this invention. Coloring agents may include titanium dioxide and/or dyes suitable for food such as those known as F. D. & C., dyes and natural coloring agents such as grape skin extract, beet red powder, beta carotene, annato, carmine, turmeric, paprika, and so forth. In one embodiment, the coloring agent is titanium dioxide.

In one embodiment, the pharmaceutical composition comprises a sweetener, such as a sugar or sorbitol, aspartame, citric acid, or any combination thereof.

In one embodiment, solid formulations of this invention may comprise flow regulators (glidants) such as: silica, for example in the form of colloidal anhydrous silica, colloidal silicon dioxide, calcium silicate, magnesium silicate, talc or any combination thereof. In one embodiment the glidant is colloidal silicon dioxide.

In one embodiment, solid formulations of this invention may comprise disintegrants such as starch, cornstarch, potato starch, alginic acid, silicon dioxide, croscarmelose sodium, crospovidone, pregelatinised starch, guar gum, sodium starch glycolate, cross-linked polyvinylpyrrolidone, sodium carboxymethylcellulose, clays (e.g. magnesium aluminum silicate), microcrystalline cellulose, alginates, gums, surfactants, effervescent mixtures, and hydrous aluminum silicate. In one embodiment, the surfactant is sodium lauryl sulfate.

In one embodiment, pharmaceutical composition/formulation of this invention comprises lubricants. Examples of lubricants include, but are not limited to magnesium stearate, calcium stearate, stearic acid, glycerylbehaptate, polyethylene glycol, ethylene oxide polymers, sodium lauryl sulfate, magnesium lauryl sulfate, sodium oleate, sodium stearyl fumarate, DL-leucine, colloidal silica, talc or any combination thereof. Lubricants generally comprise 0.5 to 7.0% of the total tablet weight. In one embodiment, the lubricant is magnesium stearate.

In one embodiment the pharmaceutical composition/formulation of this invention comprises a plasticizer, such as long-chain alcohols, ethylene glycol, propylene glycol, glycerol, sorbitol, trimethylolpropane, triethylene glycol, butanediols, pentanols such as pentaerythritol, hexanols, polyethylene glycols, polypropylene glycols, polyethylene/propylene glycols, silicones, aromatic carboxylic esters (for example dialkyl phthalates, trimellitic esters, benzoic esters, terephthalic esters) or aliphatic dicarboxylic esters (for example dialkyl adipates, sebacic esters, azelaic esters, citric and tartaric esters), fatty acid esters, such as glycerol mono-, di- or triacetate or sodium diethyl sulfosuccinate, or combinations thereof. In one embodiment, the plasticizer is sorbitol. In another embodiment, the plasticizer is sorbitol special—glycerin blend A810.

Flavors incorporated in the pharmaceutical composition may be chosen from synthetic flavor oils and flavoring aromatics and/or natural oils, extracts from plants leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. The amount of flavoring may depend on a number of factors including the organoleptic effect desired. Generally the flavoring will be present in an amount of from 0.5 to about 3.0 percent by weight based on the total tablet weight, when a flavor is used.

In some embodiments, the pharmaceutical composition of the present invention can include a preservative, antioxidant, buffering agent, acidifying agent, alkalizing agent, antibacterial agent, antifungal agent, solubility enhancing agent, complexation enhancing agent, solvent, electrolyte, salt, water, stabilizer, tonicity modifier, antifoaming agent, oil, emulsifying agent, bulking agent, cryoprotectant or a combination thereof. In one embodiment, the solubility enhancing agent is polyethylene glycol. In another embodiment, the solubility enhancing agent is polyethylene glycol 400.

In some embodiments, the term “alkalizing agent” refers to a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

In some embodiments, the term “acidifying agent” refers to a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

In some embodiments, the term “preservative” refers to a compound used to prevent the growth of microorganisms. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl or butyl parabens and others known to those of ordinary skill in the art. Particularly useful preservatives include EDTA, pentetate, and combinations thereof.

In some embodiments, the term “antioxidant” refers to an agent that inhibits oxidation and thus is used to prevent the deterioration of preparations by the oxidative process. Such compounds include, by way of example and without limitation, acetone sodium bisulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid and sodium metabisulfite and others known to those of ordinary skill in the art.

In some embodiments, the term “buffering agent” refers to a compound used to resist change in pH upon dilution or addition of acid or alkali. Such compounds include, by way of example and without limitation, acetic acid, sodium acetate, adipic acid, benzoic acid, sodium benzoate, citric acid, maleic acid, monobasic sodium phosphate, dibasic sodium phosphate, lactic acid, tartaric acid, potassium metaphosphate, potassium phosphate, monobasic sodium acetate, sodium bicarbonate, sodium tartrate and sodium citrate anhydrous and dihydrate and others known to those of ordinary skill in the art.

In some embodiments, the term “stabilizer” refers to a compound used to stabilize the therapeutic agent against physical, chemical, or biochemical process which would reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and other known to those of ordinary skill in the art.

In some embodiments, the term “bulking agent” refers to a compound used to add bulk to the lyophilized product and/or assist in the control of the properties of the formulation during lyophilization. Such compounds include, by way of example and without limitation, dextran, trehalose, sucrose, polyvinylpyrrolidone, lactose, inositol, sorbitol, dimethylsulfoxide, glycerol, albumin, calcium lactobionate, and others known to those of ordinary skill in the art.

In some embodiments, the term “cryoprotectant” refers to a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

In one embodiment, the pharmaceutical composition of this invention may include, a compound as described herein, any SARM compound, or other therapeutic agent as herein described, or any combination thereof, together with one or more pharmaceutically acceptable excipients.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the compounds or agents as herein described or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, and hard or soft gelatin capsules.

In some embodiments, an active component can be formulated into the pharmaceutical composition as a neutralized pharmaceutically acceptable salt form. Pharmaceutically acceptable salts include the acid addition salts, which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In some embodiments, the salts of Compounds I-V are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

In one embodiment, this invention is directed to a softgel capsule comprising a compound of this invention. In another embodiment, this invention is directed to a softgel capsule consisting essentially of a compound of this invention. In another embodiment, this invention is directed to a softgel capsule consisting essentially of compound S-I. In one embodiment, this invention provides a method of preparation of a softgel capsule comprising Compound S-I, as exemplified in Example 8. In another embodiment, the manufacturing process of S-I softgels pharmaceutical composition comprises the preparation of gel mass, preparation of S-I fill material, encapsulation and printing, drying, finishing, inspection and bulk packaging.

In one embodiment, softgels pharmaceutical composition is prepared by mixing gel mass materials, including, inter alia: gelatin, sorbitol, water and titanium dioxide; dissolving one or more of Compounds I-V or its isomer, polymorph, pharmaceutically acceptable salt, N-oxide, hydrate or any combination thereof, and/or a carrier, diluent, or a combination thereof, in a filler or solubilizer such as but not limited to polyethylene glycol to generate the fill material; following by encapsulation with the gel mass materials of the fill material using conventional encapsulation technology and drying using conventional drying technology. Further, washing and grading using a standard grading technology can take place in order to remove any gross over and undersized softgels.

In one embodiment, preparation of gel mass comprises mixing gel mass ingredients together according to U.S. Pat. No. 7,213,511, U.S. Pat. No. 5,735,105 and U.S. Pat. No. 6,769,226 patents which are incorporated herein by reference and manufacturing process for gel preparation. In another embodiment, the gel mass ingredients comprise gelatin, sorbitol (optionally blended with glycerin), water and titanium dioxide.

In another embodiment, preparation of active compound fill material comprises mixing a solubilizing agent with the active compound. In another embodiment, the solubilizing agent comprises polyethylene glycol (including but not limited to polyethylene glycol 400). In another embodiment, mixing takes place under vacuum until the active compound is fully dissolved. In another embodiment, following the mixing, the mixture is allowed to stand for de-aeration. In another embodiment, following de-aeration, the mixing vessel is vented with inert gas. In another embodiment, in the end of the process the mixture is stored under inert atmosphere. In another embodiment, the inert gas is nitrogen. In another embodiment, the active compound comprises compound I-V, and/or its isomer, polymorph, pharmaceutically acceptable salt, N-oxide, hydrate or any combination thereof. In another embodiment, the active compound comprises compound S-I and/or its isomer, polymorph, pharmaceutically acceptable salt, N-oxide, hydrate or any combination thereof. In another embodiment, the active compound comprises compound S-I.

In another embodiment, encapsulation of the active compound fill material is performed on a rotary die encapsulation machine. In another embodiment, the active compound comprises compound I-V, and/or its isomer, polymorph, pharmaceutically acceptable salt, N-oxide, hydrate or any combination thereof. In another embodiment, the active compound comprises compound S-I and/or its isomer, polymorph, pharmaceutically acceptable salt, N-oxide, hydrate or any combination thereof. In another embodiment, the active compound comprises compound S-I. In another embodiment, following encapsulation the outer shell of the softgel is optionally imprinted.

In another embodiment, drying of the softgel takes place in drying tunnels (tray drying). In another embodiment, the drying process takes place until the softgel required hardness is achieved. In another embodiment, the required hardness is about 9-11 Newton (N). In another embodiment, once the required hardness is achieved, the softgels are washed on a spray washer with wash solution. In another embodiment, the wash solution comprises denatured ethanol/Phosal solution.

In another embodiment, following washing grading of the softgels takes place to remove any gross over and under sized softgels. In another embodiment, grading takes place through a sizing device.

In another embodiment, following packaging, storage of the softgels takes place at 15-30° C. with no more than 50% relative humidity (RH).

In one embodiment, the pharmaceutical composition is prepared by obtaining a mixture of one or more of Compounds I-V or its isomer, polymorph, pharmaceutically acceptable salt, N-oxide, hydrate or any combination thereof, and a carrier, diluent, or a combination thereof, granulated according to conventional granulation technology and drying the granules using conventional drying technology. The dried granules may optionally be resized. In the case where the pharmaceutical composition is a capsule, the granules are filled into the capsule, (e.g. gelatin capsule). In the case the pharmaceutical composition is a tablet, the granules may be mixed with glidants/lubricants and compressed into tablets using conventional technology.

In one embodiment, granules may be produced in a manner known per se, for example using wet granulation methods known for the production of “built-up” granules or “broken-down” granules. Methods for the formation of built-up granules may operate continuously and comprise, for example simultaneously spraying the granulation mass with granulation solution and drying, for example in a drum granulator, in pan granulators, on disc granulators, in a fluidized bed, by spray-drying or spray-solidifying, or operate discontinuously, for example in a fluidized bed, in a batch mixer or in a spray-drying drum.

In another embodiment, methods for the production of broken-down granules, which may be carried out discontinuously and in which the granulation mass first forms a wet aggregate with the granulation solution, which aggregate is then comminuted or formed into granules of the desired particle size and the granules then being dried. Suitable equipment for the granulation step are planetary mixers, low and high shear mixers, wet granulation equipment including extruders and spheronisers include, for example, apparatus from the companies Loedige, Glatt, Diosna, Fielder, Collette, Aeschbach, Alexanderwerk, Ytron, Wyss & Probst, Werner & Pfleiderer, HKD, Loser, Fuji, Nica, Caleva and Gabler.

The granulation mass consists of comminuted, preferably ground, Compound I-V and the excipients mentioned above. Depending on the method used, the granulation mass may be in the form of a premix or may be obtained by mixing Compound I-V into one or more excipients or mixing the excipients into Compound I-V. The wet granules are preferably dried, for example in the described manner by tray drying or in a fluidised bed.

In some embodiments, tablet cores are produced using the so-called compacting or dry granulation method in which the active ingredient is compressed with the excipients to form relatively large mouldings, for example slugs or ribbons, which are comminuted by grinding, and the ground material is compressed to form tablet cores.

Suitable excipients for the compacting method comprise those which are suitable for the conventional direct compression methods, for example dry binders, such as starches, for example potato, wheat and maize starch, microcrystalline cellulose, for example commercial products available under the trademarks Avicel®, Filtrak®, Heweten® or Pharmacel®, highly dispersed silicon dioxide, for example Aerosil®, mannitol, lactose, and also polyethylene glycol, especially having a molecular weight of from 4000 to 6000, crosslinked polyvinylpyrrolidone (Polyplasdone® XL or Kollidon® CL), crosslinked carboxymethylcellulose (Acdisol® CMC-XL), carboxymethylcellulose [Nymcel®, for example ZSB-10, (Nyma)], hydroxypropylmethylcellulose, for example the quality HPMC 603, carboxymethyl starch [Explotab® (Mendell) or Primojel® (Scholtens)], microcrystalline cellulose, for example Avicel® PH 102, dicalcium phosphate, for example Emcompress® or talcum. The addition of small amounts of, for example, lubricants, such as magnesium stearate, is also advantageous.

Compression to form tablet cores may be carried out in conventional tabletting machines, for example EK-0 Korsch eccentric tabletting machines or rotary tabletting machines. The tablet cores may be of various shapes, for example round, oval, oblong, cylindrical etc., and various sizes, depending on the amount of Compound I-V.

In one embodiment, this invention provides a method of preparation of a tablet comprising Compound S-I, as exemplified in Example 5.

In order to produce Compound I-V particles, e.g. crystals having a desired particle size, conventional comminution and de-agglomeration techniques may be used, for example grinding in an air-jet mill or impact mill, a ball mill, vibration mill, mortar mill or pin mill.

The milling step is typically performed on particles of varying sizes, i. e., large particles, powders, and fine powders to obtain a preferred and more uniform particle size. The milling can include several separating, recycling, and screening steps to obtain the desired particle sizes.

Drying is generally performed using a suitable drying instrument selected by one of skill in the art such as a fluid bed dryer.

In one embodiment, the pharmaceutical composition prepared according to these routes can be encapsulated in a capsule or compressed into a tablet or caplet, which can optionally be encapsulated in a capsule. If the pharmaceutical composition is compressed into a tablet or caplet, the tablets or caplets can optionally be film-coated. Suitable film-coatings are known to those of skill in the art. For example, the film-coating can be selected from among suitable polymers such as hydroxpropylmethylcellulose, ethyl cellulose, polyvinyl alcohol, and combinations thereof. Other suitable film-coatings can be readily selected by one of skill in the art. In one embodiment, the tablet or caplet is coated with an Opadry® seal coat. Where applied, the weight percent of the film coat is generally in the range of 2% wt/wt to 6% wt/wt of the tablet or caplet.

In one embodiment, the capsule can be made as a hard capsule filled with a powder or granular pharmaceutical agent. The hard capsule is produced by mixing and/or granulating an active ingredient with, for example, an excipient (e.g., lactose, sucrose, starch, crystalline cellulose, D-mannitol and the like), a disintegrant (e.g., low substituted hydroxypropyl cellulose, carmellose calcium, corn starch, croscarmellose sodium and the like), an “active” excipient (e.g. polyoxyl 35 castor oil, polyoxyl 4 lauryl ether, polyoxyethylenesorbitan monolaurate, etc.), a binder (e.g., hydroxypropyl cellulose, polyvinylpyrrolidone, hydroxypropylmethyl cellulose and the like), a lubricant (e.g., magnesium stearate and the like) and the like, and filling the mixture or granule in a capsule formed from the aforementioned gelatin, pullulan, and the like. The cellulose can be hydroxymethyl cellulose, hydroxypropylmethyl cellulose, or any other form of cellulose known in the art.

In another embodiment, hard capsules are made with gelatin by a dip molding process. The dip molding process is based on the ability of hot gelatin solutions to set by cooling. For the industrial manufacture of pharmaceutical capsules, gelatin is preferred for its gelling, film forming and surface active properties. A typical dip molding process comprises the steps of dipping mold pins into a hot solution of gelatin, removing the pins from the gelatin solution, allowing the gelatin solution attached on pins to set by cooling, drying and stripping the so-formed shells from the pins. The rapid setting of the solution on the mold pins after dipping is the critical step to obtain a uniform thickness of the capsule shell. On a totally automatic industrial hard gelatin capsule machine, the pins having a coating of gelatin are turned from downside to upside to dry the gelatin solution attached on the pins. When the gelatin is cool and set, the capsule shell is stripped from the pin and subsequently cut and the cap and body are joined.

In one embodiment, this invention provides methods for preparing capsules comprising Compound I-V. In another embodiment, this invention provides methods for preparing softgel capsules comprising Compound I-V. In another embodiment, this invention provides methods for preparing hard gelatin capsules comprising Compound I-V. In one embodiment the capsules are size 5 capsules. In another embodiment the capsules are size 3 capsules. In another embodiment, the capsules are size 1 capsules. In another embodiment, the capsules are white opaque NP capsules. In another embodiment, the capsules are white opaque hard gelatin capsules. In another embodiment, the capsules are white to off-white opaque softgel capsules. In another embodiment, all drug substance is micronized using a Jet-O-Mill® Micronizer. In another embodiment, the drug substance is dissolved in a solubilizer and encapsulated using a “Rotary Die Encapsulation” process as described herein above. In another embodiment, the method of preparation capsules formulation for 0.1 mg Compound I-V comprises high shear dry blend of the active ingredient and excipients and fill into size 3 capsules. In another embodiment, the method of preparation of capsules formulation for 0.1 mg Compound I-V comprises high sheer wet granulation followed by excipient dilution and fill into size 3 capsules. In another embodiment, the method of preparation of capsules formulation for 0.1 mg Compound I-V comprises direct blend and fill into size three capsules.

In another embodiment, the method of preparation of capsules of a formulation for 0.1, 0.3 mg, 0.5 mg, 1 mg, 1.5 mg, 2 mg, 2.5 mg, 3 mg, 4 mg, 5 mg, 9 mg, 10 mg, 30 mg, 100 mg, or 300 mg of Compounds I-V comprises mixing the active ingredient together with the excipients in a blender and once formulated, the mixed components are fed into an automatic capsule filler.

In another embodiment, the method of preparation of capsules of a formulation for about 0.1 mg to about 300 mg of Compounds I-V comprises mixing the active ingredient together with the excipients in a blender and once formulated, the mixed components are fed into an automatic capsule filler. In another embodiment, the method of preparation of capsules of a formulation for about 0.1 mg to about 3 mg of Compounds I-V comprises mixing the active ingredient together with the excipients in a blender and once formulated, the mixed components are fed into an automatic capsule filler. In another embodiment, the method of preparation of capsules of a formulation for about 1 mg to about 100 mg of Compounds I-V comprises mixing the active ingredient together with the excipients in a blender and once formulated, the mixed components are fed into an automatic capsule filler. In another embodiment, the method of preparation of capsules of a formulation for about 3 mg to about 30 mg of Compounds I-V comprises mixing the active ingredient together with the excipients in a blender and once formulated, the mixed components are fed into an automatic capsule filler.

In another embodiment, methods of preparation of softgel capsules comprise dissolving the drug substance in a solubilizer or filler, e.g. polyethylene glycol 400, following by encapsulation with a gel mass comprising gelatin, sorbitol blended with glycerin, titanium dioxide and water. In one embodiment, the drug substance consists essentially of 9 mg Compound I-V. In one embodiment, the drug substance consists essentially of 5 mg Compound I-V. In one embodiment, the drug substance consists essentially of 4 mg Compound I-V. In one embodiment, the drug substance consists essentially of 3 mg Compound I-V. In another embodiment, the drug substance consists essentially of 0.1 mg Compound I-V. In another embodiment, the drug substance consists essentially of 0.3 mg Compound I-V. In another embodiment, the drug substance consists essentially of 0.5 mg Compound I-V. In another embodiment, the drug substance consists essentially of 1 mg Compound I-V. In another embodiment, the drug substance consists essentially of 1.5 mg Compound I-V. In another embodiment, the drug substance consists essentially of 2 mg Compound I-V. In another embodiment, the drug substance consists essentially of 2.5 mg Compound I-V. In another embodiment, the drug substance consists essentially of 0.1 mg, 0.3 mg, 0.5 mg, 1 mg, 1.5 mg, 2 mg, 2.5 mg, 3 mg, 4, mg, 5 mg, 9 mg, 10 mg, 30 mg, 100 mg, or 300 mg of Compounds I-V. In another embodiment, the drug substance consists essentially of 0.1 mg to about 300 mg Compounds I-V.

In one embodiment, the methods of this invention may comprise administration of pharmaceutical compositions comprising Compounds I-V at various dosages.

In one embodiment, the compound is administered at a dosage of 0.1-200 mg per day. In one embodiment, the compound is administered at a dose of 0.1-10 mg per day, or in another embodiment, 0.1-26 mg per day, or in another embodiment, 0.1-60 mg per day, or in another embodiment, 0.3-16 mg per day, or in another embodiment, 0.3-30 mg per day, or in another embodiment, 0.6-26 mg per day, or in another embodiment, 0.6-60 mg per day, or in another embodiment, 0.76-16 mg per day, or in another embodiment, 0.76-60 mg per day, or in another embodiment, 1-6 mg per day, or in another embodiment, 1-20 mg per day, or in another embodiment, 3-16 mg per day, or in another embodiment, 30-60 mg, or in another embodiment, 30-76 mg per day, or in another embodiment, 100-200 mg per day, or in another embodiment, 0.1-200 mg per day, or in another embodiment 0.01-500 mg per day.

In one embodiment, any one of Compounds I-V is administered at a dosage of 0.1 mg per day. In one embodiment, the compound is administered at a dosage of 0.3 mg per day. In one embodiment, the compound is administered at a dosage of 0.5 mg per day. In one embodiment, the compound is administered at a dosage of 1 mg per day. In one embodiment, the compound is administered at a dosage of 3 mg per day. In one embodiment, the compound is administered at a dosage of 4 mg per day. In one embodiment, the compound is administered at a dosage of 5 mg per day. In one embodiment, the compound is administered at a dosage of 9 mg per day. In one embodiment, the compound is administered at a dosage of 10 mg per day. In one embodiment, the compound is administered at a dosage of 30 mg per day. In another embodiment the compound is administered at a dosage of 0.1 mg per day, 0.3 mg per day, 0.5 mg per day, 1 mg per day, 1.5 mg per day, 2 mg per day, 3 mg per day, 4 mg per day, 5 mg per day, 9 mg per day, 10 mg per day, 30 mg per day or 100 mg per day. In another embodiment the compound is administered at a dosage of 6 mg per day, 10 mg per day, 16 mg per day, 20 mg per day, 26 mg per day, 30 mg per day, 36 mg per day, 40 mg per day, 46 mg per day, 50 mg per day, 56 mg per day, 60 mg per day, 66 mg per day, 70 mg per day, 76 mg per day, 80 mg per day, 86 mg per day, 90 mg per day, 96 mg per day or 100 mg per day.

In another embodiment, the compound is administered at a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg.

In one embodiment, the compound is administered at a dosage of 0.1-200 mg per kg. In one embodiment, the compound is administered at a dose of 0.1-10 mg per kg, or in another embodiment, 0.1-26 mg per kg, or in another embodiment, 0.1-60 mg per kg, or in another embodiment, 0.3-16 mg per kg, or in another embodiment, 0.3-30 mg per kg, or in another embodiment, 0.6-26 mg per kg, or in another embodiment, 0.6-60 mg per kg, or in another embodiment, 0.76-16 mg per kg, or in another embodiment, 0.76-60 mg per kg, or in another embodiment, 1-6 mg per kg, or in another embodiment, 1-20 mg per kg, or in another embodiment, 3-16 mg per kg, or in another embodiment, 30-60 mg per kg, or in another embodiment, 30-76 mg per kg, or in another embodiment, 100-200 mg per kg, or in another embodiment, 0.1-200 mg per kg, or in another embodiment 0.01-500 mg per kg.

In one embodiment, any one of Compounds I-V is administered at a dosage of 0.1 mg. In one embodiment, the compound is administered at a dosage of 0.3 mg. In one embodiment, the compound is administered at a dosage of 0.5 mg. In one embodiment, the compound is administered at a dosage of 1 mg. In one embodiment, the compound is administered at a dosage of 3 mg. In one embodiment, the compound is administered at a dosage of 4 mg. In one embodiment, the compound is administered at a dosage of 5 mg. In one embodiment, the compound is administered at a dosage of 9 mg. In one embodiment, the compound is administered at a dosage of 10 mg. In one embodiment, the compound is administered at a dosage of 30 mg. In another embodiment the compound is administered at a dosage of 6 mg, 10 mg, 16 mg, 20 mg, 26 mg, 30 mg, 36 mg, 40 mg, 46 mg, 50 mg, 56 mg, 60 mg, 66 mg, 70 mg, 76 mg, 80 mg, 86 mg, 90 mg, 96 mg or 100 mg.

In one embodiment, the compound of this invention is administered in multiple dosage units which results in 1 to 200 mg dose. In another embodiment, 3 mg dose is being administered using three 1 mg dosage units of this invention. In another embodiment, 3 mg dose is being administered using six 0.5 mg dosage units of this invention.

In one embodiment, this invention provides methods for preparing capsules, hard gelatin capsules, softgel capsules, and/or tablets comprising Compound S-I as exemplified in Examples 3, 4, 5, 7, 8 and 11.

The solid dosage forms of tablets, dragees, troche, capsules, powder-filled capsules, softgels, liquid-filled softgels, pills, beads and granules can be prepared with coatings and shells such as enteric coatings and other coatings well known in the pharmaceutical formulating art. They may optionally contain opacifying agents and can also be of a pharmaceutical composition that they release the active ingredient(s) only, or preferentially, in a certain part of the intestinal tract, optionally, in a delayed manner. Examples of embedding pharmaceutical composition that can be used include polymeric substances and waxes.

In one embodiment, this invention provides a pharmaceutical composition comprising Compound I-V or a SARM or other compounds as herein described. In one embodiment, such pharmaceutical composition is useful for oral testosterone replacement therapy.

In one embodiment, this invention also provides a pharmaceutical composition comprising two or more SARMs as described herein, or polymorphs, isomers, hydrates, salts, N-oxides, or any combination thereof. The present invention also relates to pharmaceutical composition, which comprises Compound I-V alone or in combination with a progestin or estrogen, or in another embodiment, chemotherapeutic compound, osteogenic or myogenic compound, or bisphosphonate, such as for example, alendronate, or other agents suitable for the applications as herein described. In one embodiment, the pharmaceutical composition of this invention will comprise a suitable carrier, diluent or salt.

In some embodiments, the methods herein comprise administration of a therapeutically effective amount of a pharmaceutical composition to achieve the desired or stated effect. Typically, the pharmaceutical composition of this invention will be administered from about 1 to about 6 times per day or alternatively, as a continuous infusion or continuous release dosage form. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active ingredient (w/w). Alternatively, such preparations may contain from about 20% to about 80% active ingredient.

Lower or higher doses than those recited above may be required. Specific dosage and treatment regimens for any particular patient will depend upon a variety of factors, including the activity of the specific pharmaceutical composition employed, the age, body weight, general health status, sex, diet, time of administration, rate of excretion, drug combination, the severity and course of the disease, condition or symptoms, the patient's disposition to the disease, condition or symptoms, and the judgment of the treating physician.

In one embodiment, the present invention provides methods of use comprising the administration of a pharmaceutical composition comprising a) any embodiment of a compound as described herein; and b) a pharmaceutically acceptable carrier or diluent; which is to be understood to include an isomer, polymorph, pharmaceutically acceptable salt, N-oxide, hydrate or any combination thereof, of a compound as herein described.

In some embodiments, the present invention provides methods of use of a pharmaceutical composition comprising a) any embodiment of the compounds as described herein, including an isomer, polymorph, pharmaceutically acceptable salt, N-oxide, hydrate or any combination thereof; b) a pharmaceutically acceptable carrier or diluent; c) a flow-aid; and d) a lubricant.

In another embodiment, the present invention provides methods of use of a pharmaceutical composition comprising a) any embodiment of the compounds as described herein, including an isomer, polymorph, pharmaceutically acceptable salt, N-oxide, hydrate or any combination thereof; b) lactose monohydrate; c) microcrystalline cellulose; d) magnesium stearate; and e) colloidal silicon dioxide.

In another embodiment, the present invention provides methods of use of a pharmaceutical composition comprising a) any embodiment of the compounds as described herein, including an isomer, polymorph, pharmaceutically acceptable salt, N-oxide, hydrate or any combination thereof; b) polyethylene glycol; c) gelatin; d) sorbitol; and e) titanium dioxide.

In some embodiments, the methods of this invention make use of pharmaceutical composition comprising Compound I-V, which offer the advantage that the compounds are nonsteroidal ligands for the androgen receptor, and exhibit anabolic activity in vivo. According to this aspect, such compounds are unaccompanied by serious side effects, provide convenient modes of administration, and lower production costs and are orally bioavailable, lack significant cross-reactivity with other undesired steroid receptors, and may possess long biological half-lives.

For administration to mammals, and particularly humans, it is expected that the physician will determine the actual dosage and duration of treatment, which will be most suitable for an individual and can vary with the age, weight and response of the particular individual.

In one embodiment, the invention provides a pharmaceutical composition, including any embodiment described herein, for use in any of the methods of this invention, as described herein. In one embodiment, use of a pharmaceutical composition comprising Compound I-V, will have utility in inhibiting, suppressing, enhancing or stimulating a desired response in a subject, as will be understood by one skilled in the art. In another embodiment, the pharmaceutical composition may further comprise additional active ingredients, whose activity is useful for the particular application for which the compound is being administered.

In some embodiments, the pharmaceutical composition will further comprise a 5α-reductase inhibitor (SARI), a SARM, a selective estrogen receptor modulator (SERM), an aromatase inhibitor (AI), such as but not limited to anastrazole, exemestane, or letrozole; a GnRH agonist or antagonist, a steroidal or nonsteroidal GR ligand, a steroidal or nonsterodial PR ligand, a steroidal or nonsteroidal AR antagonist, a 17-aldoketoreductase inhibitor or 17β-hydroxysteroid dehydrogenase inhibitor. Such a pharmaceutical composition may be used, in some embodiments, for treating a hormone dependent condition, such as, for example, infertility, neoplasia of a hormone-responsive cancer, for example, a gonadal cancer, or a urogenital cancer.

In some embodiments, the pharmaceutical composition will comprise Compound I-V, or any embodiment thereof as described herein, as well as another therapeutic compound, including inter alia, a SARI, including but not limited to, finasteride, dutasteride, izonsteride; other SARMs, including but not limited to, RU-58642, RU-56279, WS9761 A and B, RU-59063, RU-58841, bexlosteride, LG-121071, LG-121091, LG-121104, LGD-2226, LGD-2941, YM-92088, YM-175735, BMS-357597, BMS-391197, S-40503, BMS-482404, EM-4283, EM-4977, BMS-564929, BMS-391197, BMS-434588, BMS-487745, BMS-501949, SA-766, YM-92088, YM-580, LG-123303, LG-123129, YM-175735, BMS-591305, BMS-591309, BMS-665139, BMS-665539, CE-590, 116BG33, 154BG31, arcarine, ACP-105; SERMs, including but not limited to, tamoxifen, 4-hydroxytamoxifen, idoxifene, toremifene, ospemifene, droloxifene, raloxifene, arzoxifene, bazedoxifene; GnRH agonists or antagonists, including but not limited to, leuprolide, goserelin, triptorelin, alfaprostol, histrelin, detirelix, ganirelix,; FSH agonist/antagonist, LH agonist/antagonists, aromatase inhibitors, including but not limited to, letrozole, anastrazole, atamestane, fadrozole, minamestane, exemestane; Steroidal or nonsteroidal glucocorticoid receptor ligands, including but not limited to, ZK-216348, ZK-243149, ZK-243185, LGD-5552, mifepristone,; Steroidal or nonsterodial progesterone receptor ligands; Steroidal or nonsteroidal AR antagonists such as flutamide, hydroxyflutamide, bicalutamide, nilutamide, hydroxysteroid dehydrogenase inhibitors, PPARα ligand including but not limited to bezafibrate, fenofibrate, gemfibrozil; PPARγ ligands including but not limited to darglitazone, pioglitazone, rosiglitazone,; Dual acting PPAR ligands, including but not limited to naveglitazar, farglitazar, tesaglitazar; a 17-ketoreductase inhibitors, 3β-DHΔ4,6-isomerase inhibitors, 3β-DHΔ4,5-isomerase inhibitors, 17,20 desmolase inhibitors, p450c17 inhibitors, p450ssc inhibitors, 17,20-lyase inhibitors, or combinations thereof.

In some embodiments, the pharmaceutical composition will further comprise ghrelin receptor ligand or growth hormone analogues and secretagogues, IGF-1, IGF-1 analogues and secretagogues, myostatin analogues, proteasome inhibitors, androgenic/anabolic steroid, Enbrel, melanocortin 4 receptor agonist, insulins, or combinations thereof. Such a pharmaceutical composition may be used, in some embodiments, for treating sarcopenia or a musculoskeletal condition.

The invention contemplates, in some embodiments, administration of a pharmaceutical composition comprising the individual agents, administered separately and by similar or alternative routes, formulated as appropriately for the route of administration. The invention contemplates, in some embodiments, administration of a pharmaceutical composition comprising the individual agents, administered in the same formulation. The invention contemplates, in some embodiments, staggered administration, concurrent administration, of administration of the various agents over a course of time; however, their effects are synergistic in the subject.

It is to be understood that any of the above means, timings, routes, or combinations thereof, of administration of two or more agents is to be considered as being encompassed by the phrase “administered in combination”, as described herein.

In one embodiment, the pharmaceutical composition of compound I-V as herein described,comprises a compound of this invention in combination with an anti-cancer agent. In one embodiment, the anti-cancer agent is a monoclonal antibody. In some embodiments, the monoclonal antibodies are used for diagnosis, monitoring, or treatment of cancer. In one embodiment, monoclonal antibodies react against specific antigens on cancer cells. In one embodiment, the monoclonal antibody acts as a cancer cell receptor antagonist. In one embodiment, monoclonal antibodies enhance the patient's immune response. In one embodiment, monoclonal antibodies act against cell growth factors, thus blocking cancer cell growth. In one embodiment, anti-cancer monoclonal antibodies are conjugated or linked to anti-cancer drugs, radioisotopes, other biologic response modifiers, other toxins, or a combination thereof. In one embodiment, anti-cancer monoclonal antibodies are conjugated or linked to the compound as described hereinabove.

In another embodiment, the present invention includes a pharmaceutical composition in which Compound I-V is either combined with, or covalently bound to, an agent bound to a targeting agent, such as a monoclonal antibody (e.g., a murine or humanized monoclonal antibody). In one embodiment, the agent bound to a targeting agent is a cytotoxic agent. It will be appreciated that the latter combination may allow the introduction of cytotoxic agents into for example cancer cells with greater specificity. Thus, the active form of the cytotoxic agent (i.e., the free form) will be present only in cells targeted by the antibody. Of course, the compounds of the invention may also be combined with monoclonal antibodies that have therapeutic activity against cancer.

In one embodiment, the pharmaceutical composition of compound I-V as herein described, comprises a compound of this invention in combination with a selective tyrosine kinase inhibitor. In some embodiments, the selective tyrosine kinase inhibitor inhibits catalytic sites of cancer promoting receptors thereby inhibiting tumor growth. In one embodiment, a selective tyrosine kinase inhibitor modulates growth factor signaling. In some embodiments, the selective tyrosine kinase inhibitor targets EGFR (ERB B/HER) family members. In one embodiment, the selective tyrosine kinase inhibitor is a BCR-ABL tyrosine kinase inhibitor. In one embodiment, the selective tyrosine kinase inhibitor is an epidermal growth factor receptor tyrosine kinase inhibitor. In one embodiment, the selective tyrosine kinase inhibitor is a vascular endothelial growth factor tyrosine kinase inhibitor. In one embodiment, the selective tyrosine kinase inhibitor is a Platelet Derived Growth Factor (PDGF) inhibitor.

In one embodiment, the pharmaceutical composition of compound I-V as herein described, comprises a compound of this invention in combination with an anaplastic lymphoma kinase (ALK) inhibitor. The anaplastic lymphoma kinase (ALK) is a receptor tyrosine kinase that belongs to the insulin receptor superfamily and is normally expressed in neural tissues during embryogenesis. Various cytoplasmic ALK fusion proteins and the full-length ALK in its transmembrane receptor form are valid molecular targets for anticancer drugs. Consequently, a small-molecule inhibitor of ALK kinase is likely to be a drug for suppressing of tumor growth and angiogenesis. Examples of A1K inhibitors are disclosed in WO 2009/117097 and references therein which is incorporated herein by reference.

In one embodiment, the pharmaceutical composition of this invention comprises Compound I-V or other actives as herein described, in combination with a cancer vaccine. In one embodiment, the cancer vaccine is a therapeutic vaccine thus, treating an existing cancer. In some embodiments, the cancer vaccine is a prophylactic vaccine thus, preventing the development of cancer. In one embodiment, both types of vaccines have the potential to reduce the burden of cancer. In one embodiment, treatment or therapeutic vaccines are administered to cancer patients and are designed to strengthen the body's natural defenses against cancers that have already developed. In one embodiment, therapeutic vaccines may prevent additional growth of existing cancers, prevent the recurrence of treated cancers, or eliminate cancer cells not killed by prior treatments. In some embodiments, prevention or prophylactic vaccines are administered to healthy individuals and are designed to target cancer in individuals who present high risk for the disease. In one embodiment, the cancer vaccine is an antigen/adjuvant vaccine. In one embodiment, the cancer vaccine is a whole cell tumor vaccine. In one embodiment, the cancer vaccine is a dendritic cell vaccine. In one embodiment, the cancer vaccine comprises viral vectors and/or DNA vaccines. In one embodiment, the cancer vaccine is an idiotype vaccine.

In one embodiment, the pharmaceutical composition of this invention comprises Compound I-V or embodiments thereof or other actives as herein described, in combination with an anti-cancer chemotherapeutic agent. In one embodiment, the anti-cancer chemotherapeutic agent is an alkylating agent, such as but not limited to cyclophosphamide. In one embodiment, the anti-cancer chemotherapeutic agent is a cytotoxic antibiotic such as but not limited to doxorubicin. In one embodiment, the anti-cancer chemotherapeutic agent is an antimetabolite, such as but not limited to methotrexate. In one embodiment, the anti-cancer chemotherapeutic agent is a vinca alkaloid, such as but not limited to vindesine. In some embodiments, the anti-cancer chemotherapeutic agents include platinum compounds such as but not limited to carboplatin, and taxanes such as docetaxel. In one embodiment, the anti-cancer chemotherapeutic agent is an aromatase inhibitor such as but not limited to anastrazole, exemestane, or letrozole.

In one embodiment, the pharmaceutical composition of this invention comprises Compound I-V or embodiments thereof or other actives as herein described, in combination with a Bax activity modulator such as alisol B acetate. In one embodiment, the pharmaceutical composition of this invention comprises

In one embodiment, the pharmaceutical composition of this invention comprises Compound I-V or embodiments thereof or other actives as herein described, in combination with a vaccine for prostate cancer, Alisol B acetate, angiotensin II receptor blocker, or others known in the art.

In one embodiment, the pharmaceutical composition of this invention comprises Compound I-V or embodiments thereof or other actives as herein described, in combination with an immunomodulating agent. In one embodiment, the immunomodulating agent is an immunosuppressive agent. In one embodiment, immunosuppressive agents comprise corticosteroids, cyclosporine, azathioprine, methotrexate, cyclophosphamide, tacrolimus or FK-506, anti-thymocyte globulin, mycophenylate moeftil, or a combination thereof. In one embodiment, the corticosteroid is a glucocorticoid.

In one embodiment, the immunomodulating agent is an immunostimulatory agent. In one embodiment, the immunostimulatory agent is a specific immunostimulator thus, provides antigenic specificity during an immune response, such as a vaccine or any antigen. In one embodiment, the immunostimulatory agent is a non-specific immunostimulator thus, acting irrespective of antigenic specificity to augment immune response of other antigen or stimulate components of the immune system without antigenic specificity.

In some embodiments, the pharmaceutical composition of this invention comprises the Compound I-V or embodiments thereof or other actives as herein described, in combination with an anti-inflammatory agent. In one embodiment, the immunomodulating agent is an anti-inflammatory agent. In one embodiment, the anti-inflammatory agent is a non-steroidal anti-inflammatory agent. In one embodiment, the non-steroidal anti-inflammatory agent is a cox-1 inhibitor. In one embodiment, the non-steroidal anti-inflammatory agent is a cox-2 inhibitor. In one embodiment, the non-steroidal anti-inflammatory agent is a cox-1 and cox-2 inhibitor. In some embodiments, non-steroidal anti-inflammatory agents include but are not limited to aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib.

In one embodiment, the pharmaceutical composition of this invention comprises Compound I-V or embodiments thereof or other actives as herein described, in combination with an antidiabetic agent. In one embodiment, the antidiabetic agent is a sulfonylurea. In one embodiment, sulfonylureas include but are not limited to tolbutamide, acetohexamide, tolazamide, chlorpropamide, glipizide, glyburide, glimepiride, or gliclazide. In one embodiment, the antidiabetic agent is a meglitinide. In one embodiment, meglitinides include but are not limited to Prandin or nateglinide. In one embodiment, the antidiabetic agent is a biguanide. In one embodiment, biguanides include but are not limited to metformin In one embodiment, the antidiabetic agent is a thiazolidinedione. In one embodiment, thiazolidinediones include but are not limited to rosiglitazone, pioglitazone, or troglitazone. In one embodiment, the antidiabetic agent is an alpha glucosidase inhibitor. In one embodiment, alpha glucosidase inhibitors include but are not limited to miglitol or acarbose. In one embodiment, the antidiabetic agent is PPARα/γ ligand, dipeptidylpeptidase 4 (DPP-4) inhibitor, SGLT (sodium-dependent glucose transporter 1) inhibitor, or FBPase (fructose 1,6-bisphosphatase) inhibitor. In one embodiment, the antidiabetic agent is insulin. In one embodiment, the insulin is rapid-acting insulin. In one embodiment, the insulin is short-acting insulin. In one embodiment, the insulin is intermediate-acting insulin. In one embodiment, the insulin is intermediate- and short-acting insulin mixtures. In one embodiment, the insulin is long-acting insulin. In one embodiment, the antidiabetic agents are inhibitors of fatty acid binding protein (aP2) such as those disclosed in U.S. Ser. No. 09/519,079 filed Mar. 6, 2000, glucagon-like peptide-1 (GLP-1), and dipeptidyl peptidase IV (DPP4) inhibitors such as those disclosed in WO 0168603, which are incorporated by reference.

In one embodiment, the antidiabetic agent is a glucokinase activator. In one embodiment, the antidiabetic agent is a fructose-1,6-bisphosphatase inhibitor. In one embodiment, the antidiabetic agent is a glycogen phosphorylase inhibitor. In one embodiment, the antidiabetic agent is a Na⁺/glucose cotransporter (SGLT) inhibitor. In one embodiment, the antidiabetic agent is a beta3-adrenergic receptor agonist. In one embodiment, the antidiabetic agent is an adipocyte fatty acid binding protein (aFABP or aP2) ligand. In one embodiment, the antidiabetic agent is a hormone sensitive lipase inhibitor. In one embodiment, the antidiabetic agent is a GPR40 (FFAR1) ligand. In one embodiment, the antidiabetic agent is a CETP inhbitor. In one embodiment, the antidiabetic agent is a insulin-like growth factor 1 receptor modulator. In one embodiment, the antidiabetic agent is an insulin receptor modulator. In one embodiment, the antidiabetic agent is a glucocorticoid receptor antagonist. In one embodiment, the antidiabetic agent is a retinoic acid X receptor (RXR) modulator. In one embodiment, the antidiabetic agent is a liver X receptor (LXR) modulator. In one embodiment, the antidiabetic agent is a farnesyl X receptor (FXR) modulator. In one embodiment, the antidiabetic agent is a phosphotyrosine phosphatase type 1B (PTP-1B (PTPN1)) inhibitor. In one embodiment, the antidiabetic agent is an AMP-activated protein kinase (AMPK) activator. In one embodiment, the antidiabetic agent is a glycogen synthase kinase-3 (GSK3beta) inhibitor. In one embodiment, the antidiabetic agent is a 11beta-hydroxysteroid dehydrogenase type 1 (11β-HSD1) inhibitor. In one embodiment, the antidiabetic agent is an inhibitor of inhibitor kappaB kinase (IKKbeta). In one embodiment, the antidiabetic agent is a ghrelin receptor or growth hormone secretagogue receptor ligand. In one embodiment, the antidiabetic agent is a glucagon receptor ligand. In one embodiment, the antidiabetic agents are modulators of resistin or adiponectin. In one embodiment, the antidiabetic agent is an inhibitor of triacylglyercol lipases or lipid phosphorylases. In one embodiment, the antidiabetic agent is a c-jun N-terminal kinase (JNK) inhibitor. In one embodiment, the antidiabetic agent is a melanocortin receptor (MC4R) modulator.

In one embodiment, the pharmaceutical composition of this invention comprises the compound I-V or embodiments thereof or other actives as herein described, in combination with an agent treating the nervous system. In one embodiment, the agent treating the autonomic nervous system is an adrenomimetic drug. In one embodiment, the adrenomimetic drug is a beta-adrenoceptor agonist, alpha-adrenoceptor agonist, or a combination thereof. In one embodiment, the adrenomimetic drug is a catecholamine.

In one embodiment, the agent treating the autonomic nervous system is an adrenoceptor antagonist. In one embodiment, the adrenoceptor antagonist is a haloalkylamine, imidazoline, quinazoline, phenoxybenzamine. In one embodiment, imidazolines, combined alpha and beta blocking activity, cholinomimetic agent, or a cholinesterase inhibitor. In one embodiment, the inhibitor targets acetylcholine in the central nervous system such as tacrine, donepezil, or galanthamine. In one embodiment, the agent treating the autonomic nervous system is a muscarinic blocking agent, ganglionic blocking agent.

In one embodiment, the agent treating the nervous system is an agent treating the central nervous system. In one embodiment, the agent treating the central nervous system is a local anesthetic agent, a general anaesthetic agent or combinations thereof.

In one embodiment, the agent treating the central nervous system is an analgesic agent, including but not limited to paracetamol or non-steroidal anti-inflammatory agent, opiates or morphinomimetics, or combinations thereof.

In one embodiment, the agent treating the central nervous system is a muscle relaxant, a vasoconstrictor agent a muscle relaxant, including but are not limited to methocarbamol, baclofen, carisoprodol, chlorzoxazone, cyclobenzaprine, dantrolene, metaxalone, orphenadrine, amyl nitrite, pancuronium, tizanidine, clonidine, or gabapentin. In one embodiment, vasoconstrictor agents include but are not limited to antihistamines, adrenalin dimethylarginine, caffeine, cannabis, catecholamines, decongestants, pseudoephedrinse, norepinephrines, tetrahydrozoline, or thromboxane.

In one embodiment, the agent treating the central nervous system is an antiemetic drug, a dopamine antagonist, an antihistamine such as cyclizine, diphenhydramine, dimenhydrinate, or meclizine or a cannabinoid such as cannabis or marinol a sedative, an antidepressant,a barbiturate, a benzodiazepine, an imidazopyridines, an antihistamine an antipsychotic, an herbal sedative such as valerian plant mandrake, or kava. In some embodiments, the sedative agent is eszopiclone, ramelteon, methaqualone, ethchlorvynol, chloral hydrate, meprobamate, glutethimide, methyprylon, gamma-hydroxybutyrate, ethyl alcohol, methyl trichloride, zopiclone, or diethyl ether.

In one embodiment, the agent treating the central nervous system is a neurodegenerative disorder medication, an acetylcholinesterase, an N-methyl-D-aspartate (NMDA) antagonist such as memantine. In one embodiment, the neurodegenerative disorder medication reduces damage to motor neurons such as riluzole. In one embodiment, the neurodegenerative disorder medication silences the gene that causes the progression of the disease. In one embodiment, the agent treating the central nervous system is an antiepileptic drug (AED), sodium channel blockers, GABA receptor agonists, GABA reuptake inhibitors, GABA transaminase inhibitor, AEDs with a potential GABA mechanism of action, glutamate blockers, carbamazepine, fosphenytoin, oxcarbazepine, lamotrigine, zonisamide, clobazam, clonazepam, phenobarbital, primidone, tiagabine, vigabatrin, gabapentin, valproate, felbamate, topiramate, levetiracetam, or pregabalin.

In one embodiment, the agent treating the central nervous system is an anti-addiction drug, an anti-alcoholism drug such as disulfiram, a serotonin uptake inhibitor, dopaminergic agonist, or opioid antagonist.

In one embodiment, the pharmaceutical composition of this invention comprises Compound I-V or embodiments thereof or other actives as herein described, in combination with a behavior-modulating agent. In some embodiments, behavior-modulating agents include but are not limited to an anti-anxiety agent, anti-psychotic agent, anti-depressant, beta-blocker, beta-2 agonist, anticholinergic bronchodilator, theophylline, aminophylline, nedocromil sodium, sodium cromoglycate, leukotriene receptor antagonist, corticosteroid, expectorant, mucolytic agent, antihistamine, pseudoephedrine, methylphenidate, amphetamine, buspirone, benzodiazepine, dextroamphetamine, tricyclic antidepressant, serotonin reuptake inhibitor, phenothiazines, benztropine, bupropion, propranolol, lithium, venlafaxine, haloperidol, buspirone, a neuraminidase inhibitor, a benzodiazepine, a phenothiazine, a tricyclic antidepressant or a serotonin reuptake inhibitor.

In one embodiment, the pharmaceutical composition of this invention comprises Compound I-V or embodiments thereof or other actives as herein described, in combination with an agent treating the cardiovascular system. In one embodiment, the agent treating the cardiovascular system is treating a congestive heart failure. In one embodiment, the agent treating congestive heart failure is an angiotensin converting enzyme inhibitor (ACE inhibitor, a beta-blocker, digoxin, a diuretic including but not limited to thiazide diuretic, loop diuretic, potassium-sparing diuretic, or a combination thereof. In some embodiments, potassium-sparing diuretics include but are not limited to amiloride, triamterene, aldosterone antagonists, or spironolactone.

In one embodiment, the agent treating the cardiovascular system is an anti-arrhythmic agent. In one embodiment, the anti-arrhythmic agent is a sodium channel blocker, beta-adrenergic blocker, calcium channel blocker, or an agent that prolong repolarization.

In one embodiment, the agent treating the cardiovascular system is an anti-anginal agent. In one embodiment, the anti-anginal agent is an antiplatelet agent, adrenoceptor antagonist, calcium channel blocker, or a vasodilator. In some embodiments, the adrenoceptor antagonists and calcium channel blockers comprise agents as described hereinabove. In one embodiment, the antiplatelet agent is a cyclooxygenase inhibitor, ADP inhibitor, phosphodiesterase III inhibitor, glycoprotein IIb/IIIa inhibitor, or an adenosine reuptake inhibitor. In one embodiment, cyclooxygenase inhibitors include but are not limited to acetylsalicylic acid or an acetylsalicylic acid in combination with dipyridimole. In one embodiment, ADP inhibitors include but are not limited to clopidogrel, CS-747, or ticlopdipine. In one embodiment, phosphodiesterase III inhibitors include but are not limited to cilostazol. In one embodiment, glycoprotein IIb/IIIa inhibitors include but are not limited to abciximab, rheopro, eptifibatide, integrilin, tirofiban, or aggrastat. In one embodiment, adenosine reuptake inhibitors include but are not limited to dipyridimole. In one embodiment, vasodilator agents include but are not limited to isosorbide dinitrate, isosorbide mononitrate, or nitroglycerine. In one embodiment, cardiac glycosides such as digitalis or ouabain may be used in combination with a SARM compound.

In one embodiment, the agent treating the cardiovascular system is a vasoactive agent or an inotrope. In one embodiment, vasocative agents or inotropes include but are not limited to digoxin, dopamine, dobutamine, hydralazine, nitroprusside, nitroglycerin, captopril, nifedipine, diltiazem, furosemide, spironolactone, AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), or nitrates.

In one embodiment, the agent treating the cardiovascular system is an anticoagulant agent. In one embodiment, the anticoagulant agent is a coumarin derivative or an unfractionated heparin. In one embodiment, the anticoagulant agent is a fractionated heparin. In one embodiment, coumarin derivatives include but are not limited to warfarin.

In one embodiment, the agent treating the cardiovascular system is a fibrinolytic agent such as streptokinase, urokinase, alteplase, anistreplase, prourokinase, reteplase, tenecteplase, lanoteplase, staphylokinase, vampire, or alfimeprase.

In one embodiment, the agent treating the cardiovascular system is a hypercholesterolemic agent such as niacin-lovastatin, colestipol HCl, fluvastatin sodium, atorvastatin calcium, simvastatin, gemfibrozil, lovastatin, pravastatin sodium, cholestyramine, cholestyramine light, fenofibrate, colesevelam HCl, or ezetimibe.

In one embodiment, the pharmaceutical composition of this invention comprises Compound I-V or embodiments thereof or other actives as herein described, in combination with an agent treating the gastrointestinal system. In one embodiment, the agent treating the gastrointestinal (GI) system is enhancing GI motility. In one embodiment, the agent enhancing GI motility is a prokinetic agent such as metoclopramide, cisapride, tegaserod, or erythromycin. In one embodiment, the agent treating the GI system is decreasing GI motility.

In one embodiment, the agent decreasing GI motility is an opioid such as morphine, diphenoxylate, loperamide hydrochloride, or opium.

In one embodiment, the agent treating the GI system is an adsorbent or a bulking agent, a stool softener, a laxative, a cathartic stimulant, an emetic agent. In one embodiment, the emetic agent is a 5-HT₃ antagonist such as ondansetron or granisetron. In one embodiment, the agent treating the GI system is an antacid, an H2-receptor antagonist, a proton pump inhibitor, an agent treating inflammation. In one embodiment, the agent treating inflammation is 5-amino-salicylate, corticosteroid, metronidazole, ciprofloxacin, infiximab, budesonide, or anti-TNF alpha antibody.

In one embodiment, the SARM compound is administered in combination with an agent treating a dermatological disorder.

In one embodiment, the agent treating a dermatological disorder is photochemotherapy agent. In one embodiment, the photochemotherapy agent is PUVA or psoralen such as oxsoralen.

In one embodiment, the agent treating a dermatological disorder is daspone, thalidomide, anti-malarial agent, antimicrobial agent, antiviral, antihistamine, immunosuppressant, antifungal agent, or an antibiotic systemic, or topical, as disclosed herein.

In one embodiment, the agent treating a dermatological disorder is treating pigmentation such as hydroquinone or monobenzone or a protein or a recombinant protein such as becaplermin, etanercept, denileukin diftitox, or botulinum toxin. In one embodiment, the agent treating a dermatological disorder is capsaicin, anthralin, benzoyl peroxide, or calcipotriene.

In one embodiment, the agent treating a dermatological disorder is a keratolytic, a growth factor such as epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), or any combination thereof.

In one embodiment, the pharmaceutical composition of this invention comprises Compound I-V or embodiments thereof or other actives as herein described, in combination with an anti-infective agent. In one embodiment, the anti-infective agent is an antibiotic agent. In one embodiment the antibiotic is a beta-lactam antibiotic. In one embodiment beta-lactam antibiotics include but are not limited to penicillin, amoxicillin, dicloxacillin ampicillin, methicillin, azlocillin, carbenicillin, ticarcillin, mezlocillin, piperacillin, co-amoxiclav, cephalosporin, imipenem, meropenem, ertapenem, faropenem, monobactam, aztreonam, or carbapenem.

In one embodiment the antibiotic is a tetracycline antibiotic, a macrolide antibiotic, an aminoglycoside antibiotic, a quinolone antibiotic, a cyclic peptide antibiotic including but are not limited to vancomycin, streptogramins, Microcin J25, Bacteriocin AS-48, RTD-1, or polymyxins.

In one embodiment the antibiotic is a lincosamide antibiotic, an oxazolidinone antibiotic, a sulfa antibiotic, or an antiseptic agent.

In one embodiment, the pharmaceutical composition of this invention comprises Compound I-V or embodiments thereof or other actives as herein described, in combination with an agent treating the kidney. In one embodiment, the agent treating the kidney is a diuretic.

In one embodiment, the agent treating the kidney is erythropoietin. In one embodiment, erythropoietin is obtained by natural sources (e.g., urinary erythropoietin; See U.S. Pat. No. 3,865,801), or is a recombinantly produced protein and analogs thereof, for example, as described in U.S. Pat. Nos. 5,441,868, 5,547,933, 5,618,698 and 5,621,080 as well as human erythropoietin analogs with increased glycosylation and/or changes in the amino acid sequence as those described in European Patent Publication No. EP 668351 and the hyperglycosylated analogs having 1-14 sialic acid groups and changes in the amino acid sequence described in PCT Publication No. WO 91/05867. In one embodiment, erythropoietin-like polypeptides are administered in combination with SARM compounds. In some embodiments, erythropoietin-like polypeptides comprise darbepoietin (from Amgen; also known as Aranesp and novel erthyropoiesis stimulating protein (NESP)).

In one embodiment, the pharmaceutical composition of this invention comprises Compound I-V or embodiments thereof or other actives as herein described, in combination with an agent treating a wasting disease. In other embodiments, agents treating a wasting disease may comprise growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or, in other embodiments, with growth hormone releasing factor and its analogs or growth hormone and its analogs, or with alpha-adrenergic agonists, such as clonidine or serotinin 5-HTD agonists, such as sumatriptan, or agents which inhibit somatostatin or its release, such as physostigmine and pyridostigmine. In some embodiments, agents treating a wasting disease may comprise parathyroid hormone, PTH(1-34) or bisphosphonates, such as MK-217 (alendronate). In other embodiments, agents treating wasting disease may further comprise estrogen, a selective estrogen receptor modulator, such as tamoxifene or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999). In some embodiments, agents treating a wasting disease may further comprise a progesterone receptor agonists (“PRA”), such as levonorgestrel, medroxyprogesterone acetate (MPA). In some embodiments, agents treating a wasting disease may include nutritional supplements, such as those described in U.S. Pat. No. 5,179,080, which, in other embodiments are in combination with whey protein or casein, amino acids (such as leucine, branched amino acids and hydroxymethylbutyrate), triglycerides, vitamins (e.g., A, B₆, B₁₂, folate, C, D and E), minerals (e.g., selenium, magnesium, zinc, chromium, calcium and potassium), carnitine, lipoic acid, creatinine, β-hyroxy-β-methylbutyriate (Juven®) and coenzyme Q. In one embodiment, agents treating a wasting disease may further comprise antiresorptive agents, vitamin D analogues, elemental calcium and calcium supplements, cathepsin K inhibitors, MMP inhibitors, vitronectin receptor antagonists, Src SH₂ antagonists, vacular-H⁺-ATPase inhibitors, ipriflavone, fluoride, tibolone, prostanoids, 17-beta hydroxysteroid dehydrogenase inhibitors and Src kinase inhibitors.

In one embodiment, growth promoting agents such as but not limited to TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890 are utilized as agents treating a wasting disease.

In one embodiment, the pharmaceutical composition of this invention comprises Compound I-V or embodiments thereof or other actives as herein described, in combination with an inhibitor of an enzyme involved in the androgen biosynthetic pathway. In some embodiments, inhibitors of enzymes involved in the androgen biosynthetic pathway include but are not limited to 17-ketoreductase inhibitor, 3-ΔH4,6-isomerase inhibitor, 3-ΔH4,5-isomerase inhibitor, 17,20 desmolase inhibitor, p450c17 inhibitor, p450ssc inhibitor, or 17,20-lyase inhibitor.

In one embodiment, the pharmaceutical composition of this invention comprises Compound I-V or embodiments thereof or other actives as herein described, in combination with an agent treating pharmacotherapy induced hypogonadal and/or osteopenic and/or sarcopenic state. In some embodiments, agents treating pharmacotherapy induced hypogonadal and/or osteopenic and/or sarcopenic states include but are not limited to opioids, narcotics, opiates, opioids, methadone, Kadian®, D₂ dopamine receptor antagonist, zotepine, haloperidol, amisulpride, risperidone, anti-epileptic agent, valproic acid, carbamazepine, oxcarbamazepine, chemotherapeutic agent, methotrexate, cyclophosphamide, ifosfamide, adriamycin, doxorubicin, glucocorticoids, cyclosporine, L-thyroxine, SERMs, aromatase inhibitor, fulvestrant, gonadotropin-releasing hormone agent, androgen depravation agent, prolactinemia-inducing agent, serotonergic antidepressant, selective serotonin reuptake inhibitor, monoamine oxidase inhibitor, tricyclic antidepressant, antihypertensive agents, methyldopa, reserpine, clonidine, verapamil, antidopaminergic agent, anti-emetic agent, metoclopramide, H₂ receptor antagonist, cimetidine, ranitidine, estrogen, or amphetamine.

In one embodiment, the pharmaceutical composition of this invention comprises Compound I-V or embodiments thereof or other actives as herein described, in combination with an agent treating a connective tissue. In some embodiments, agents treating a connective tissue include but are not limited to an anti-malaria agent, a cytotoxic agent, a steroid, corticosteroid, lupus medication, Imuran®, Cytoxan®, anti-rheumatic agent, corticosteroid, nifedipine, aspirin, colchicine, captopril, penicillamine, azathioprine, methotrexate, cyclophosphamide, prednisone, nicardipine, or a non-steroidal anti-inflammatory agent.

In one embodiment, the anti-rheumatic agent is a corticosteroid. In one embodiment, the corticosteroid is prednisone or dexamethasone. In one embodiment, the anti-rheumatic agent is a disease modifying anti-rheumatic drug. In one embodiment, the disease modifying anti-rheumatic drug is a slow-acting anti-rheumatic drug. In one embodiment, the disease modifying anti-rheumatic drug is an antimalarial agent. In one embodiment, disease modifying anti-rheumatic drugs include but are not limited to chloroquine, hydroxychloroquine, methotrexate, sulfasalazine, cyclosporine, azathioprine, cyclophosphamide, azathioprine, sulfasalazine, penicillamine, aurothioglucose, gold sodium thiomalate, or auranofin. In one embodiment, the anti-rheumatic agent is an immunosuppressive cytotoxic drug. In one embodiment, immunosuppressive cytotoxic drugs include but are not limited to methotrexate, mechlorethamine, cyclophosphamide, chlorambucil, or azathioprine.

In one embodiment the pharmaceutical composition of this invention comprises Compound I-V or embodiments thereof or other actives as herein described, in combination with an agent treating an ophthalmic disease. In some embodiments, agents treating an ophthalmic disease include but are not limited to betagan, betimol, timoptic, betoptic, betoptic, ocupress, optipranolol, Xalatan®, Alphagan®, Azopt®, Trusopt®, Cosopt®, Pilocar®, Pilagan®, Propine, Opticrom®, Acular®, Livostin®, Alomide, Emadine, Patanol®, Alrex®, Poly-Pred®, Pred-G®, Dexacidin, Erythromycin, Maxitrol®, Tobradex®, Blephamide®, FML®, Ocufen®, Voltaren®, Profenal, Pred Forte®, Econpred Plus®, Eflone®, Flarex®, Inflamase Forte®, betadine, gramicidin, prednisolone, betaxolol, humorsol, proparacaine, Betoptic®, Hylartin, Inflamase Mild®, Lotemax®, flurbiprofen, chloramphenicol, cyclosporine, methazolamide, timolol, Ciloxan®, terramycin, ciprofloxacin, Miostat, triamcinolone, miconazole, tobramycin, physostimine, gentamicin, pilocarpine, bacitracin, goniosol, polymyxin, oxytetracycline, Viroptic®, Vexol®, Suprofen®, Celluvisc®, Polytrim, Illotycin, Ciloxan®, Ocuflox®, brinzolamide, cefazolin, Tobrex®, latanoprost, indocycanine, trifluridine, phenylephrine, demecarium, neomycin, tropicamide, dexamethasone, neptazane, dipivefrin, vidarabine, dorzolamide, ofloxacin, epinephrine, acyclovir, carbonic anhydrase inhibitor, antihistamine, vitamin A, vitamin C, vitamin E, zinc, copper, atropine,or garamycin.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, the combined preparations can be varied, e.g., in order to cope with the needs of a patient subpopulation to be treated or the needs of the single patient which different needs can be due to the particular disease, severity of the disease, age, sex, or body weight as can be readily determined by a person skilled in the art. In some embodiments, the methods of the present invention comprise personalized medicine methods which treat the needs of a single patient. In one embodiment, different needs can be due to the particular disease, severity of the disease, the overall medical state of a patient, or the age of the patient. In some embodiments, personalized medicine is the application of genomic data to better target the delivery of medical interventions. Methods of personalized medicine, in some embodiments, serve as a tool in the discovery and clinical testing of new products of the present invention. In one embodiment, personalized medicine involves the application of clinically useful diagnostic tools that may help determine a patient's predisposition to a particular disease or condition. In some embodiments, personalized medicine is a comprehensive approach utilizing molecular analysis of both patients and healthy individuals to guide decisions throughout all stages of the discovery and development of pharmaceuticals and diagnostics; and applying this knowledge in clinical practice for a more efficient delivery of accurate and quality healthcare through improved prevention, diagnosis, treatment, and monitoring methods.

It is to be understood that this invention is directed to a pharmaceutical composition and combined therapies as described herein, for any disease, disorder or condition, as appropriate, as will be appreciated by one skilled in the art. Certain applications of such a pharmaceutical composition and combined therapies have been described hereinabove, for specific diseases, disorders and conditions, representing embodiments of this invention, and methods of treating such diseases, disorders and conditions in a subject by administering a SARM as herein described, alone or as part of the combined therapy or using the pharmaceutical composition of this invention represent additional embodiments of this invention.

### Applications of the Pharmaceutical Compositions of this Invention

In one embodiment, this invention provides a pharmaceutical composition as herein described, comprising Compound I-V and/or an isomer, polymorph, pharmaceutically acceptable salt, N-oxide, hydrate or any combination thereof, for a) treating a bone related disorder; b) preventing a bone related disorder; c) suppressing a bone related disorder; d) inhibiting a bone related disorder; e) increasing a strength of a bone of a subject; f) increasing a bone mass in a subject; g) use for osteoclastogenesis inhibition; h) accelerating bone repair; i) treating bone density loss; j) treating low bone mineral density (BMD); k) treating reduced bone mass; 1) treating metabolic bone disease; m) promoting bone growth or regrowth; n) promoting bone restoration; o) promoting bone fracture repair; p) promoting bone remodeling; q) treating bone damage following reconstructive surgery including of the face, hip, or joints; r) enhancing of bone strength and function; s) increasing cortical bone mass; and t) increasing trabecular connectivity. In another embodiment, the pharmaceutical composition comprises Compound I-V. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg,5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a liquid-filled capsule (softgel capsule) comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the bone related disorder is a genetic disorder, or in another embodiment, is induced as a result of a treatment regimen for a given disease. For example, and in one embodiment, the SARMs and/or a pharmaceutical composition comprising the same as herein described are useful in treating a bone-related disorder that arises as a result of cancer metastasis to bone, or in another embodiment, as a result of androgen-deprivation therapy, for example, given in response to prostate carcinogenesis in the subject.

In one embodiment, the bone-related disorder is osteoporosis. In another embodiment, the bone-related disorder is osteopenia. In another embodiment, the bone-related disorder is increased bone resorption. In another embodiment, the bone-related disorder is bone fracture. In another embodiment, the bone-related disorder is bone frailty. In another embodiment, the bone-related disorder is a loss of bone mineral density (BMD). In another embodiment, the bone-related disorder is any combination of osteoporosis, osteopenia, increased bone resorption, bone fracture, bone frailty and loss of BMD. Each disorder represents a separate embodiment of the present invention.

“Osteoporosis” refers, in one embodiment, to a thinning of the bones with reduction in bone mass due to depletion of calcium and bone protein. In another embodiment, osteoporosis is a systemic skeletal disease, characterized by low bone mass and deterioration of bone tissue, with a consequent increase in bone fragility and susceptibility to fracture. In osteoporotic patients, bone strength is abnormal, in one embodiment, with a resulting increase in the risk of fracture. In another embodiment, osteoporosis depletes both the calcium and the protein collagen normally found in the bone, in one embodiment, resulting in either abnormal bone quality or decreased bone density. In another embodiment, bones that are affected by osteoporosis can fracture with only a minor fall or injury that normally would not cause a bone fracture. The fracture can be, in one embodiment, either in the form of cracking (as in a hip fracture) or collapsing (as in a compression fracture of the spine). The spine, hips, and wrists are common areas of osteoporosis-induced bone fractures, although fractures can also occur in other skeletal areas. Unchecked osteoporosis can lead, in another embodiment, to changes in posture, physical abnormality, and decreased mobility.

In one embodiment, the osteoporosis results from androgen deprivation. In another embodiment, the osteoporosis follows androgen deprivation. In another embodiment, the osteoporosis is primary osteoporosis. In another embodiment, the osteoporosis is secondary osteoporosis. In another embodiment, the osteoporosis is postmenopausal osteoporosis. In another embodiment, the osteoporosis is juvenile osteoporosis. In another embodiment, the osteoporosis is idiopathic osteoporosis. In another embodiment, the osteoporosis is senile osteoporosis.

In another embodiment, the primary osteoporosis is Type I primary osteoporosis. In another embodiment, the primary osteoporosis is Type II primary osteoporosis. Each type of osteoporosis represents a separate embodiment of the present invention.

In another embodiment, the methods of the present invention comprise administering a pharmaceutical composition as herein described comprising Compound I-V and/or an isomer, polymorph, pharmaceutically acceptable salt, N-oxide, hydrate or any combination thereof, in combination with calcitonin such as salmon, Elcatonin, SUN-8577 or TJN-135 for treating osteoporosis.

In another embodiment, the methods of treating osteoporosis of the present invention comprise administering a pharmaceutical composition as herein described comprising Compound I-V and/or an isomer, polymorph, pharmaceutically acceptable salt, N-oxide, hydrate or any combination thereof, in combination with a) vitamin D or derivative such as ZK-156979; b) vitamin D receptor ligand and analogues such as calcitriol, topitriol, ZK-150123, TEI-9647, BXL-628, Ro-26-9228, BAL-2299, Ro-65-2299 or DP-035; c) estrogen, estrogen derivative, or conjugated estrogens; d) antiestrogen, progestins, or synthetic estrogen/progestins; e) RANK ligand mAb such as denosumab formerly AMG162 (Amgen); f) ανβ3 Integrin receptor antagonist; g) osteoclast vacuolar ATPase inhibitor; h) antagonist of VEGF binding to osteoclast receptors; i) calcium receptor antagonist; j) PTh (parathyroid hormone) and analogues, PTHrP analogues (parathyroid hormone-related peptide); k) cathepsin K inhibitors (AAE581, etc.); 1) strontium ranelate; m) tibolone; n) HCT-1026, PSK3471; o) gallium maltolate; p) nutropin AQ; q) prostaglandins (for osteo); r) p38 protein kinase inhibitor; s) bone morphogenetic protein; t) inhibitor of BMP antagonism; u) HMG-CoA reductase inhibitor; v)vitamin K or derivative; w) ipriflavone; x) fluoride salts; y) dietary calcium supplement, and z) osteoprotegerin.

In some embodiments, the pharmaceutical composition of this invention comprises the Compound I-V or embodiments thereof or other actives as herein described, in combination with a SERM, as herein described, a bisphosphonate, for example, alendronate, pamidronate, etidronate, alendronate, zolendronate, cimadronate, neridronate, minodronic acid, ibandronate, risedronate, homoresidronate, a calcitonin, for example, salmon, Elcatonin, calcitriol, topitriol, gallium maltolate; Nutropin AQ; prostaglandins, p38 protein kinase inhibitor; a bone morphogenetic protein; an inhibitor of BMP antagonism, an HMG-CoA reductase inhibitor, or any combination thereof. In one embodiment, the pharmaceutical composition of this invention comprises Compound I-V as herein described, in combination with alendronate.

According to this aspect of the invention and in one embodiment, the bone-related disorder is treated with a pharmaceutical composition as herein described, comprising Compound I-V and/or an isomer, polymorph, pharmaceutically acceptable salt, N-oxide, hydrate or any combination thereof. In another embodiment, other bone-stimulating compounds can be provided to the subject, prior to, concurrent with or following administration of a SARM or SARMs and/or pharmaceutical composition comprising the same as herein described. In one embodiment, such a bone stimulating compound may comprise natural or synthetic materials.

In one embodiment, the bone stimulating compound may comprise a bone morphogenetic protein (BMP), a growth factor, such as epidermal growth factor (EGF), a fibroblast growth factor (FGF), a transforming growth factor (TGF, an insulin growth factor (IGF), a platelet-derived growth factor (PDGF) hedgehog proteins such as sonic, indian and desert hedgehog, a hormone such as follicle stimulating hormone, parathyroid hormone, parathyroid hormone related peptide, activins, inhibins, follistatin, frizzled, frzb or frazzled proteins, BMP binding proteins such as chordin and fetuin, a cytokine such as IL-3, IL-7, GM-CSF, a chemokine, such as eotaxin, a collagen, osteocalcin, osteonectin and others, as will be appreciated by one skilled in the art.

In another embodiment, the pharmaceutical composition for use in treating a bone disorder of this invention may comprise a SARM or SARMs and/or a pharmaceutical composition comprising the same as herein described, an additional bone stimulating compound, or compounds, and osteogenic cells. In one embodiment, an osteogenic cell may be a stem cell or progenitor cell, which may be induced to differentiate into an osteoblast. In another embodiment, the cell may be an osteoblast. In another embodiment, nucleic acids which encode bone-stimulating compounds may be administered to the subject, which is to be considered as part of this invention.

In one embodiment, this invention provides for the treatment, prevention, suppression or inhibition of, or the reduction of the risk of developing a skeletal-related event (SRE), such as pathologic fractures, hypercalcemia, bone-related pain, bone fractures, surgery of the bone, radiation of the bone, spinal cord compression, new bone metastasis, bone loss, or a combination thereof in a subject with cancer, comprising administering to a subject Compound I-V and/or its isomer, polymorph, pharmaceutically acceptable salt, N-oxide, hydrate or any combination thereof, or a pharmaceutical composition comprising the same as herein described. The invention relates, inter alia to treatment of an SRE with the pharmaceutical composition comprising Compound I-V in a subject with prostate cancer undergoing or having undergone androgen deprivation therapy (ADT). In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the skeletal-related events treated using the methods provided herein and/or utilizing the pharmaceutical composition provided herein, are fractures, which in one embodiment, are pathological fractures, non-traumatic fractures, vertebral fracture, non-vertebral fractures, morphometric fractures, or a combination thereof. In some embodiments, fractures may be simple, compound, transverse, greenstick, or comminuted fractures. In another embodiment, the pharmaceutical composition comprises Compound I-V. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment, the softgel capsule comprises Compound S-I. In another embodiment the softgel capsule consisting essentially of 3 mg of Compound S-I.

In another embodiment, the invention provides a pharmaceutical composition as herein described, comprising Compound I-V and/or an isomer, polymorph, pharmaceutically acceptable salt, N-oxide, hydrate or any combination thereof, for the treatment, prevention, suppression, inhibition or reduction of the risk of skeletal-related events such as pathologic fractures, spinal cord compression, hypercalcemia, bone-related pain, or their combination.

In another embodiment, the skeletal-related events sought to be treated using the methods provided herein and/or utilizing the pharmaceutical composition provided herein, comprise the necessity for bone surgery and/or bone radiation, which in some embodiments, is for the treatment of pain resulting in one embodiment from bone damage, or nerve compression. In another embodiment, the skeletal-related events sought to be treated using the methods provided herein and/or utilizing the pharmaceutical composition provided herein, comprise spinal cord compression, or the necessity for changes in antineoplastic therapy, including changes in hormonal therapy, in a subject. In some embodiments, skeletal-related events sought to be treated using the methods provided herein and/or utilizing the pharmaceutical composition provided herein, comprise treating, suppressing, preventing, reducing the incidence of, or delaying progression or severity of bone metastases, or bone loss. In one embodiment, bone loss may comprise osteoporosis, osteopenia, or a combination thereof. In one embodiment, skeletal-related events may comprise any combination of the embodiments listed herein.

In one embodiment, the skeletal-related events are a result of cancer therapy. In one embodiment, the skeletal-related events are a result of hormone deprivation therapy, while in another embodiment, they are a product of androgen deprivation therapy (ADT).

In one embodiment, the methods provided herein and/or utilizing the pharmaceutical composition provided herein, are effective in reducing metastases to the bone, such as in terms of number of foci, the size of foci, or a combination thereof.

A person skilled in the art would readily recognize that changes in the antineoplastic therapy according to the methods provided herein, utilizing the pharmaceutical composition provided herein may be conducted as a function of, or adjusted or varied as a function of, inter alia, the severity of the underlying disease, the source of the underlying disease, the extent of the patients' pain and source of the patients' pain, as well as the stage of the disease. The therapeutic changes may include in certain embodiments, changes in the route of administration (e.g. intracavitarily, intraarterially, intratumorally etc.), forms of the pharmaceutical composition administered (e.g. tablets, elixirs, suspensions etc.), changes in dosage and the like. Each of these changes is well recognized in the art and is encompassed by the embodiments provided herein.

In one embodiment, the pharmaceutical composition of this invention is useful in prevention or reversal of androgen-deprivation therapy (ADT) induced side effects such as reduced muscle mass, reduced muscle strength, frailty, hypogonadism, osteoporosis, osteopenia, decreased BMD and/or decreased bone mass.

In males, while the natural decline in sex-hormones at maturity (direct decline in androgens as well as lower levels of estrogens derived from peripheral aromatization of androgens) is associated with the frailty of bones, this effect is more pronounced in males who have undergone androgen deprivation therapy.

In one embodiment, any of the methods of this invention or pharmaceutical composition/compounds as described herein are useful or applicable in a subject, which is a human. In another embodiment, the subject is a mammal. In another embodiment the subject is an animal. In another embodiment the subject is an invertebrate. In another embodiment the subject is a vertebrate. In one embodiment, the subject is male. In another embodiment, the subject is female. In some embodiments, while the methods as described herein may be useful for treating either males or females, females may respond more advantageously to administration of certain compounds, for certain methods, as will be appreciated by one skilled in the art. In some embodiments, while the methods as described herein may be useful for treating either males or females, males may respond more advantageously to administration of certain compounds, for certain methods, as described herein.

In another embodiment of the present invention, a method is provided for hormonal therapy in a patient (i.e., one suffering from an androgen-dependent condition) which includes contacting an androgen receptor of a patient with a pharmaceutical composition comprising compound I-V as herein described, and/or its isomer, polymorph, pharmaceutically acceptable salt, N-oxide, hydrate or any combination thereof, in an amount effective to bind the SARM compound to the androgen receptor and effect a change in an androgen-dependent condition. In another embodiment the pharmaceutical composition comprises capsules comprising 0.1 mg, 0.3 mg, 0.5 mg, 1 mg, 1.5 mg, 2 mg, 2.5, 3 mg, 4 mg, 5 mg, 9 mg,10 mg or 30 mg of Compound I-V.

In another embodiment, the pharmaceutical composition comprises Compound S-I. In another embodiment the pharmaceuticalcomposition is a capsule comprising 0.1 mg, 0.3 mg, 1 mg, 3 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment of this invention, a method is provided for hormone replacement therapy in a patient (i.e., one suffering from an androgen-dependent condition) which includes administering a pharmaceutical composition comprising compound I-V as herein described, and/or its isomer, polymorph, pharmaceutically acceptable salt, N-oxide, hydrate or any combination thereof, to a subject, in an amount sufficient to effect a change in a hormone-dependent condition in the subject. In another embodiment the pharmaceutical composition comprises capsules comprising 0.1 mg, 0.3 mg, 1 mg, 3 mg, 4 mg, 5 mg, 9 mg,10 mg or 30 mg of Compound I-V. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the hormonal disorder, disruption or imbalance comprises an excess of a hormone. In another embodiment, the hormonal disorder, disruption or imbalance comprises a deficiency of a hormone. In one embodiment, the hormone is a steroid hormone. In another embodiment, the hormone is an estrogen. In another embodiment, the hormone is an androgen. In another embodiment, the hormone is a glucocorticoid. In another embodiment, the hormone is a cortico-steroid. In another embodiment, the hormone is Luteinizing Hormone (LH). In another embodiment, the hormone is Follicle Stimulating Hormone (FSH). In another embodiment, the hormone is any other hormone known in the art. In another embodiment, the hormonal disorder, disruption or imbalance is associated with menopause. In another embodiment, the hormonal disorder, disruption or imbalance is associated with andropause, andropausal vasomotor symptoms, andropausal gynecomastia, muscle strength and/or function, bone strength and/or function and anger. In another embodiment, hormone deficiency is a result of specific manipulation, as a byproduct of treating a disease or disorder in the subject. For example, the hormone deficiency may be a result of androgen depletion in a subject, as a therapy for prostate cancer in the subject. Each possibility represents a separate embodiment of the present invention.

In one embodiment, androgen-dependent conditions which may be treated with the compounds and/or pharmaceutical composition as herein described, comprising the methods of the present invention, include those conditions which are associated with aging, hypogonadism, sarcopenia, diminished erythropoiesis, osteoporosis, and any other conditions dependent upon low androgen (e.g., testosterone) or estrogen levels.

In one embodiment, androgen-dependent conditions which may be treated with the compounds and/or pharmaceutical composition as herein described, and comprising a method of the invention, may include conditions characterized by elevated androgen or estrogen levels, including hirsutism, infertility, polycystic ovarian syndrome, endometrial carcinoma, breast cancer, male pattern baldness, prostate cancer, testicular cancer, and others, as will be known to one skilled in the art. For such conditions, the subject may be administered a pharmaceutical composition comprising compound I-V as herein described and/or an isomer, polymorph, pharmaceutically acceptable salt, pharmaceutical product, hydrate, N-oxide or any combination thereof, alone or in combination with another therapeutic agent, as will be appreciated by one skilled in the art.

In some embodiments, this invention provides methods for the treatment or reduction of the incidence of prostate cancer in a subject, comprising the step of administering to the subject a pharmaceutical composition comprising a Compound I-V as herein described and/or an isomer, polymorph, pharmaceutically acceptable salt, pharmaceutical product, hydrate, N-oxide or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, this invention provides methods for the treatment of a precancerous precursor or lesion in a subject, or reduction of incidence of precancerous precursors or lesions in a subject, comprising the step of administering to the subject a pharmaceutical composition comprising a Compound I-V as herein described and/or an isomer, polymorph, pharmaceutically acceptable salt, pharmaceutical product, hydrate, N-oxide or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, such precancerous precursors are found in hormone-responsive tissue or are associated with reproductive tissue in males or females, such as in the prostate, ovary, breast, uterus, testicle, or others. In some embodiments, such precancerous precursors comprise any local intraepithelial neoplasia, for example, of the prostate, the cervix, etc. In some embodiments, such methods are useful in treating neoplasia or pre-neoplasia, dysplasia or hyperplasia in a tissue, such as in reproductive tissue in males or females.

In one embodiment, this invention provides a pharmaceutical composition for use and/or methods of use thereof in treating benign prostate hyperplasia (BPH). “BPH (benign prostate hyperplasia)” is a nonmalignant enlargement of the prostate gland.

In another embodiment of the present invention, the method for treating benign prostate hyperplasia (BPH) in a subject, comprises the step of administering to the subject a pharmaceutical composition comprising Compound I-V as herein described and/or its isomer, polymorph, pharmaceutically acceptable salt, N-oxide, hydrate or any combination thereof, in an amount effective to treat BPH in the subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg,10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides for the use of a pharmaceutical composition as herein described, comprising Compound I-V and/or its isomer, polymorph, pharmaceutically acceptable salt, N-oxide, hydrate or any combination thereof, for 1) treating a muscle wasting disorder; 2) preventing a muscle wasting disorder; 3) treating, preventing, suppressing, inhibiting or reducing muscle loss due to a muscle wasting disorder; 4) treating, preventing, inhibiting, reducing or suppressing muscle wasting due to a muscle wasting disorder; and/or 5) treating, preventing, inhibiting, reducing or suppressing muscle protein catabolism due to a muscle wasting disorder; and/or treating, preventing, inhibiting, reducing or suppressing end stage renal disease; and/or 6) treating, preventing, inhibiting, reducing or suppressing fraility.

In another embodiment the invention is directed to treating sarcopenia or cachexia, and associated conditions related thereto, for example diseases or disorders of the bone.

The prevalence of cachexia increases from 50 percent at presentation to more than 80 percent before death from malignancy. In over 20 percent of cancer patients, cachexia is the cause of death (Bruera E. Anorexia, cachexia and nutrition. Br Med J 1997; 315: 1219-1222). Cancer cachexia leads to shorter survival, decreased response rates and increased toxicity to chemotherapy, weakness, and an overall decreased quality of life (DeWys et al. Prognostic effect of weight loss prior to chemotherapy in cancer patients. Eastern Cooperative Oncology Group. Am J Med. 1980 October; 69(4):491-7).

In one embodiment, this invention provides: a) a method of treating a subject having a muscle wasting disorder associated with cancer, cachexia, kidney disease, disuse or surgery; b) a method of treating a subject suffering from sarcopenia; c) a method of increasing muscle mass in a subject; d) a method of decreasing fat mass in a subject; e) a method of improving functional performance as measure by stair climbing (time and power) in a subject; f) a method of increasing bone mineral density in a subject; and g) a method of reducing lipid profile in a subject, comprising the step of administering to said subject a pharmaceutical composition as herein described comprising compound I-V and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, the present invention provides a method for treating, reducing the incidence, delaying the onset or progression, or reducing and/or abrogating the symptoms associated with a cancer cachexia in a subject, comprising the step of administering to the subject a pharmaceutical composition as herein described comprising Compound I-V and/or its isomer, polymorph, pharmaceutically acceptable salt, N-oxide, hydrate or any combination thereof, in an amount effective to treat, reduce the incidence, delay the onset or progression, or reduce and/or abrogate the symptoms associated with cancer cachexia in said subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, the present invention provides a method for increasing total lean body mass in a subject, comprising the step of administering to the subject a pharmaceutical composition as herein described comprising Compound I-V and/or its isomer, polymorph, pharmaceutically acceptable salt, N-oxide, hydrate or any combination thereof, in an amount effective to increase total lean body mass in said subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, the present invention provides a method for improving the functional performance of a subject, comprising the step of administering to the subject a pharmaceutical composition as herein described comprising Compound I-V and/or its isomer, pharmaceutically acceptable salt, N-oxide, hydrate or any combination thereof, in an amount effective to improve the functional performance of said subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In another embodiment, the improvement in the functional performance of said subject is measured by stair climbing speed and power.

In some embodiments, the present invention provides a method for treating, reducing the incidence, delaying the onset or progression, or reducing and/or abrogating the symptoms associated with a wasting disease in a subject, comprising the step of administering to the subject a pharmaceutical composition as herein described comprising Compound I-V and/or its isomer, polymorph, pharmaceutically acceptable salt, N-oxide, hydrate or any combination thereof, in an amount effective to treat the wasting disease in the subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, wasting diseases comprise muscle injury, bed rest, immobility, nerve injury, neuropathy, diabetic neuropathy, alcoholic neuropathy, anorexia, anorexia nervosa, anorexia associated with cachexia, anorexia associated with aging, subacute combined degeneration of the spinal cord, diabetes, rheumatoid arthritis, motor neurone diseases, Duchenne muscular dystrophy, carpal tunnel syndrome, chronic infection, tuberculosis, Addison's disease, adult sma, limb muscle atrophy, alcoholic neuropathy, anorexia nervosa, back tumour, dermatomyositis, hip cancer, inclusion body myositis, incontinentia pigmenti, intercostal neuralgia, juvenile rheumatoid arthritis, Legg-Calve-Perthes disease, muscle atrophy, multifocal motor neuropathy, nephrotic syndrome, osteogenesis imperfecta, post-polio syndrome, rib tumor, spinal muscular atrophy, reflex sympathetic dystrophy syndrome, or Tay -Sachs.

In some embodiments, the present invention provides a method for treating, reducing the incidence, delaying the onset or progression, or reducing and/or abrogating the symptoms associated with a sarcopenic state in a subject, said method comprises the step of administering to said subject a pharmaceutical composition as herein described comprising compound I-V and/or its isomer, polymorph, pharmaceutically acceptable salt, N-oxide, hydrate or any combination thereof. In one embodiment, the present invention provides a method for treating, reducing the incidence, delaying the onset or progression, or reducing and/or abrogating the symptoms associated with a pharmacotherapy induced sarcopenic state in a subject. In some embodiments, sarcopenia is a significant loss of muscle mass. In one embodiment, sarcopenia definition is having a lean body mass less than two standard deviation below the mean for normal young adults. In some embodiments, sarcopenia is caused by genetic factors, altered circulation, decrease in the capillary:muscle fiber ratio, altered motor neurons, denervation, deterioration of motor end plates, selective reinnervation of Type I fibers, inflammatory responses causing muscle damage, reduced exercise, malnutrition, low dietary protein intake, vitamin D deficiency, age-related decline in vitamin D, oxidative stress, muscle mitochondrial mutations, changes in specific types of muscle fibers, decline in muscle protein, disabling disease, strokes, Alzheimer's disease, Parkinson's disease, osteoporsis, atherosclerosis, diabetes mellitus, hyperinsulimemia, renal failure, or hypogonadism. In one embodiment, the method comprises administering to a subject compound I-V and/or a pharmaceutical composition comprising the same as herein described and an anti-cancer agent, an immunomodulating agent, an antidiabetic agent, an agent treating the cardiovascular system, an agent treating the gastrointestinal system, an agent treating the central nervous system, an agent treating a metabolic disease, an agent treating a wasting disease, a gene therapy agent, an agent treating the endocrine system, an agent treating a dermatological disorder, an anti-infective agent, an agent treating the liver, an agent treating the kidney, vitamins, or a combination thereof.

In one embodiment, the agent treating the endocrine system is a peroxisome proliferator-activated receptor ligand. In some embodiments, peroxisome proliferator-activated receptor ligands include but are not limited to bezafibrate, fenofibrate, gemfibrozil, darglitazone, pioglitazone, rosiglitazone, isaglitazone, rivoglitazone, netoglitazone, naveglitazar, farglitazar, tesaglitazar, ragaglitazar, oxeglitazar, or PN-2034.

In one embodiment, this invention provides a method of treating atherosclerosis and its associated diseases, such as, for example, cardiovascular disorders, cerebrovascular disorders, peripheral vascular disorders, or intestinal vascular disorders in a subject, the method comprising the step of administering to the subject Compound I-V and/or its isomer, polymorph, pharmaceutically acceptable salt, N-oxide, hydrate or any combination thereof, or a pharmaceutical composition comprising the same as herein described. In one embodiment atherosclerosis refers to a slow, complex disease that may begin with damage to the innermost layer of the artery. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the invention provides a method of treating, preventing, reducing the risk of mortality from cardiovascular and/or cerebrovascular disease in a subject, comprising administering a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment cardiovascular disorders comprise of hypertention (HTN), coronary artery disease (CAD) or myocardial perfusion. In another embodiment this invention provides methods of use of the pharmaceutical composition as herein described for promoting aortic smooth muscle cell proliferation. In another embodiment this invention provides methods of use of the pharmaceutical composition as herein described for treating arteriosclerosis. In another embodiment this invention provides methods of use of the pharmaceutical composition as herein described for lowering blood pressure. In another embodiment this invention provides methods of use of the pharmaceutical composition as herein described for treating cardiac diseases and disorders comprising cardiomyopathy, cardiac dysfunctions such as, myocardial infarction, cardiac hypertrophy and cognitive heart failure. In another embodiment this invention provides methods of use of the pharmaceutical composition as herein described for cardioprotection comprising cardioprotection in insulin resistance; treating diabetes type I ans II, metabolic syndrome, syndrome X and/or high blood pressure.

In one embodiment, this invention provides a method of improving the dexterity and movement in a subject, for example, by treating arthritis in the subject, comprising administering a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

The term “arthritis” refers, in another embodiment, to a non-inflammatory degenerative joint disease occurring chiefly in older people, characterized by degeneration of the articular cartilage, hypertrophy of bones and the margins, changes in the synovial membrane, etc. It is accompanied, in other embodiments, by pain and stiffness, particularly after prolonged activity.

In one embodiment, a pharmaceutical composition as herein described is useful in a) treating, preventing, suppressing, inhibiting, or reducing obesity; b) promoting, increasing or facilitating weight loss; c) decreasing, suppressing, inhibiting or reducing appetite; d) altering the body composition; e) altering lean body mass or fat free body mass; f) converting fat mass to lean mass; g) treating, preventing, suppressing, inhibiting, or reducing an obesity-associated metabolic disorder, for example hypertension, osteoarthritis, diabetes mellitus, maturity onset diabetes of the young (MODY), increased blood pressure, stroke, or heart disease; h) decreasing, suppressing, inhibiting or reducing adipogenesis; i) altering stem cell differentiation; and/or j) altering the level of leptin.

In addition, androgens have recently been shown to be involved in commitment of mesenchymal pluripotent cells into myogenic lineage and to block differentiation into adipogenic lineage (Singh et al. , Endocrinology, 2003, Jul. 24). Accordingly, a pharmaceutical composition as herein described can be useful in methods of blocking adipogenesis, and/or altering stem cell differentiation, as described herein.

In another embodiment, this invention relates to a method of decreasing, suppressing, inhibiting or reducing adipogenesis in a subject, comprising the step of administering to the subject a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In another embodiment, this invention relates to a method of altering stem cell differentiation in a subject, comprising the step of administering to the subject a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In another embodiment, this invention relates to a method of altering body composition of a subject, comprising the step of administering to the subject a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, altering the body composition comprises altering the lean body mass, the fat body mass of the subject, or a combination thereof.

In another embodiment, this invention relates to a method of altering lean body mass or fat body mass of a subject, comprising the step of administering to the subject pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the present invention provides for the use of a pharmaceutical composition as herein described for reducing a fat mass in a subject. In another embodiment, the subject has a hormonal imbalance, disorder, or disease. In another embodiment the subject has menopause.

In another embodiment, this invention relates to a method of converting fat mass to lean mass in a subject, comprising the step of administering to the subject a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In another embodiment, this invention relates to a method of treating, preventing, suppressing, inhibiting or reducing an obesity-associated metabolic disorder in a subject, comprising the step of administering to the subject a pharmaceutical composition as herein described comprising Compound I-V and/or an isomer, polymorph, pharmaceutically acceptable salt, N-oxide, hydrate or any combination thereof. In another embodiment, the pharmaceutical composition comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the obesity-associated metabolic disorder is hypertension. In another embodiment, the disorder is osteoarthritis. In another embodiment, the disorder is Type II diabetes mellitus. In another embodiment, the disorder is increased blood pressure. In another embodiment, the disorder is stroke. In another embodiment, the disorder is heart disease.

The term “diabetes”, in one embodiment, refers to a relative or absolute lack of insulin leading to uncontrolled carbohydrate metabolism. Most patients can be clinically classified as having either insulin-dependent diabetes mellitus (IDDM or Type-I diabetes) or non-insulin-dependent diabetes mellitus (NIDDM or Type-II diabetes).

The term “increased blood pressure” or “hypertension” refers, in other embodiments, to a repeatedly high blood pressure above 140 over 90 mmHg Chronically-elevated blood pressure can cause blood vessel changes in the back of the eye, thickening of the heart muscle, kidney failure, and brain damage.

The term “stroke” refers, in other embodiments, to damage to nerve cells in the brain due to insufficient blood supply often caused by a bursting blood vessel or a blood clot. The term “heart disease”, in other embodiments, refers to a malfunction in the heart normal function and activity, including heart failure.

In another embodiment, this invention provides for the use of a pharmaceutical composition as herein described comprising Compound I-V and/or its isomer, polymorph, pharmaceutically acceptable salt, N-oxide, hydrate or any combination thereof, for treating adominal fat accumulation; improving body composition; lowering body fat content; lowering fat mass; improving blood lipid profile, increasing muscle mass/strength/function; increasing bone mass/BMD/strength/function; lowering body fat; congenital hyperinsulinemia; cushing's disease (hypercortisolemia); obesity or diabetes associated with a metabolic syndrome in a subject. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of improving the lipid profile and/or reducing the circulating lipid levels in a subject, said method comprising administering to the subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the subject suffers from atherosclerosis and its associated diseases, premature aging, Alzheimer's disease, stroke, toxic hepatitis, viral hepatitis, peripheral vascular insufficiency, renal disease, hyperglycemia, or any combination thereof.

In one embodiment, compound I-V and a pharmaceutical composition comprising the same reduce LDL and total cholesterol levels. In one embodiment, Compound I-V reduces LDL and total cholesterol levels. In another embodiment, the pharmaceutical composition comprising Compound I-V reduces LDL and total cholesterol levels in a subject. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In another embodiment, compound I-V and/or a pharmaceutical composition comprising the same as herein described are co-administered with HDL-elevating agents. In another embodiment, HDL-elevating agents include niacin. In another embodiment the HDL-elevating agents include fibrates including gemfibrozil (Lopid), thiourea based gemfibrozil analogues, and fenofibrate (TriCor). In another embodiment, HDL-elevating agents include statins. In another embodiment, HDL-elevating agents include 1-hydroxyalkyl-3-phenylthiourea, and analogs thereof. In another embodiment, the pharmaceutical composition comprises Compound S-I.

In some embodiments the term “cholesterol” refers to one or more, whole cholesterol levels, LDL levels, VLDL levels, triglyceride levels and HDL levels or to the ratio of the LDL/HDL levels stayed in the normal range. In some embodiments, “cholesterol” refers to cholesterol plaques which may be present in the intima or walls of arteries in a subject. In another embodiment, “cholesterol” refers to foam cells.

In one embodiment, the pharmaceutical composition as herein described finds utility in treating or halting the progression of, or treating symptoms of diabetes. In another embodiment, the pharmaceutical composition as herein described is useful in treating co-morbidities related to diabetes.

In one embodiment this invention provides a method of treating, suppressing, inhibiting or reducing the incidence of (a) diabetes type I; (b) diabetes type II; (c) glucose intolerance; (d) hyperinsulinemia; (e) insulin resistance (f) nephropathy; (g) diabetic neuropathy; (h) diabetic retinopathy (i) fatty liver conditions (j) MODY and (k) cardiovascular disease in a human subject, said method comprises the step of administering to said subject a pharmaceutical composition comprising Compound I-V and/or its isomer, polymorph. pharmaceutically acceptable salt, N-oxide, hydrate or any combination thereof, as herein described. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating suppressing, inhibiting or reducing the incidence of diabetes is a human subject, comprising the step of administering Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In another embodiment, the diabetes is a Type I diabetes. In another embodiment, the diabetes is a type II diabetes.

In one embodiment, this invention provides a method of treating diabetes type II. Type II diabetes is characterized by insulin resistance and at some stage in pathogenesis of the disease, a relative deficiency of insulin secretion. In absolute terms, the plasma insulin concentration (both fasting and meal-stimulated) usually is increased, although “relative” to the severity of insulin resistance, the plasma insulin concentration is insufficient to maintain normal glucose homeostasis. With time, however, there is progressive beta cell failure and absolute insulin deficiency ensues. Most individuals with type II diabetes exhibit intra abdominal (visceral) obesity, fatty liver, which is closely related to the presence of insulin resistance. The patient's liver becomes insulin resistant and glycogen breakdown is uncontrolled and the result is increased and unphysiological glucose delivery to the bloodstream. The liver generated cholesterol and VLDL particles is also uncontrolled. In addition, hypertension, dyslipidemia (high triglyceride and low HDL-cholesterol levels; postprandial hyperlipemia), and elevated PAI-1 levels often are present in these individuals. This clustering of abnormalities is referred to as the “insulin resistance syndrome”, or the “metabolic syndrome” or obesity related disorders. Because of these abnormalities, patients with type II diabetes are at increased risk of developing macrovascular complications such as myocardial infarction and stroke. In one embodiment, this invention provides a method of treating diabetic nephropathy. In one embodiment, this invention provides a method of treating diabetic neuropathy. In one embodiment, this invention provides a method of treating diabetic retinopathy.

In one embodiment, this invention provides a method of treating a human subject having glucose intolerance, comprising the step of administering Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating a hyperinsulinemia in a human subject, comprising the step of administering to said subject a pharmaceutical composition comprising Compound I-V or its isomer, polymorph, pharmaceutically acceptable salt, pharmaceutical product, hydrate, N-oxide, or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating insulin resistance in a human subject, comprising the step of administering Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating diabetic nephropathy in a human subject, said method comprises the step of administering to said subject a pharmaceutical composition of the selective androgen receptor modulator Compound I-V or its isomer, polymorph, pharmaceutically acceptable salt, pharmaceutical product, hydrate, N-oxide, or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating diabetic retinopathy in a human subject, said method comprises the step of administering to said subject a pharmaceutical composition comprising Compound I-V or its isomer, polymorph, pharmaceutically acceptable salt, pharmaceutical product, hydrate, N-oxide, or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, this invention provides a method of treating fatty liver conditions in a human subject, said method comprises the step of administering to said subject a pharmaceutical composition comprising Compound I-V or its isomer, polymorph, pharmaceutically acceptable salt, pharmaceutical product, hydrate, N-oxide, or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the pharmaceutical composition as described herein is useful in prevention of iatrogenic effects comprising acute fatigue syndrome (post-surgical) or androgen-deprivation therapy (ADT) induced side effects such as reduced muscle mass, reduced muscle strength, frailty, hypogonadism, osteoporosis, osteopenia, decreased BMD and/or decreased bone mass.

In some embodiments, the pharmaceutical composition as described herein may be used for applications and treating diseases in which the improvement of cognition, reduction or treatment of depression, or other neuroportective effects are desired. In another embodiment the pharmaceutical composition comprises capsules comprising 0.1 mg, 0.3 mg, 0.5 mg, 1 mg, 1.5 mg, 2 mg, 2.5 mg, 3 mg, 10 mg or 30 mg of Compound I-V. In another embodiment, the capsules comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, the pharmaceutical composition as described herein may be used for applications and treating diseases in which the improvement of cognition, reduction or treatment of depression, or other neuroprotective effects are desired. In another embodiment the pharmaceutical composition comprises capsules comprising 0.1 mg, 0.3 mg, 0.5 mg, 1 mg, 1.5 mg, 2 mg, 2.5 mg, 3 mg, 10 mg or 30 mg of Compound I-V. In another embodiment, the capsules comprise Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, “cognition” refers to the process of knowing, specifically the process of being aware, knowing, thinking, learning and judging. Cognition is related to the fields of psychology, linguistics, computer science, neuroscience, mathematics, ethology and philosophy. In one embodiment, “mood” refers to a temper or state of the mind As contemplated herein, alterations mean any change for the positive or negative, in cognition and/or mood.

In one embodiment, “depression” refers to an illness that involves the body, mood and thoughts that affects the way a person eats, sleeps and the way one feels about oneself, and thinks about things. The signs and symptoms of depression include loss of interest in activities, loss of appetite or overeating, loss of emotional expression, an empty mood, feelings of hopelessness, pessimism, guilt or helplessness, social withdrawal, fatigue, sleep disturbances, trouble concentrating, remembering, or making decisions, restlessness, irritability, headaches, digestive disorders or chronic pain.

In some embodiments, the pharmaceutical composition as herein described may be used for applications in and/or treating diseases and/or conditions associated with problems with a subject's libido, or erectile dysfunction in a subject. In one embodiment, “libido”, may refer to sexual desire.

In one embodiment, the term “erectile” refers to the ability to be erect or upright. An erectile tissue is a tissue, which is capable of being greatly dilated and made rigid by the distension of the numerous blood vessels, which it contains.

In one embodiment, the pharmaceutical composition as described herein is useful in treating inflammation and related disorders such as: a) prevention, treatment, or reversal of arthritis; b) prevention, treatment, or reveral of an arthritic condition such as Behcet's disease (autoimmune vasculitis), bursitis, calcium pyrophosphate dihydrate crystal (CPPD), deposition disease (or pseudogout), carpal tunnel syndrome, connective tissue disorders, Crohn's dieases, Ehlers-Danlos syndrome (EDS), fibromyalgia, gout, infectious arthritis, inflammatory bowel disease (IBD), juvenile arthritis, systemic lupus erythematosus (SLE), Lyme's disease, Marfan syndrome, myositis, osteoarthritis, polyarteritis nodosa, polymyalgia rheumatica, psoriasis, psoriatic arthritis, Raynaud's phenomenon, reflex sympathetic dystrophy syndrome, Reiter's syndrome, rheumatoid arthritis, scleroderma, Sjogrens' syndrome, tendonitis or ulcerative colitis; c) preventing, treatment, or reversing an autoimmune disease.

In some embodiments, the present invention provides a method for treating, reducing the incidence, delaying the onset or progression, or reducing and/or abrogating the symptoms associated with a behavior mechanism in a subject. In some embodiments, behavior mechanisms comprise aggression, attitude to death, codependency, self-injurious behavior, sexual behavior, or social behavior.

In one embodiment, the pharmaceutical composition as herein described alters the levels of leptin in a subject.

In some embodiments, the present invention provides a method for treating, reducing the incidence, delaying the onset or progression, or reducing and/or abrogating the symptoms associated with a digestive system disease in a subject. In one embodiment, the method comprises administering to a subject a pharmaceutical composition comprising Compound I-V as herein described and an anti-cancer agent, an immunomodulating agent, an antidiabetic agent, an agent treating the central nervous system, an agent treating the gastrointestinal system, an anti-infective agent, an agent treating a metabolic disease, a gene therapy agent, an agent treating the endocrine system, vitamins, or a combination thereof.

In some embodiments, gastrointestinal diseases comprise, but is not limited to, adenomatous polyposis coli, barrett esophagus, biliary atresia, cholangitis, cholecystitis, cholelithiasis, colitis, ulcerative, Crohn's diseaseduodenal ulcer, enterocolitis, pseudomembranosusfecal incontinence, gastritis, gastritis, hypertrophic, gastroenteritis, gastroesophageal reflux, gastroparesis, hemorrhoids, hepatitis, irritable bowel syndrome, lactose intolerance, liver cirrhosis, liver diseases, meckel diverticulum, pancreatic diseases, pancreatic neoplasms, pancreatitis, or Zollinger-Ellison syndrome.

In some embodiments, the present invention provides a method for treating, reducing the incidence, delaying the onset or progression, or reducing and/or abrogating the symptoms associated with a dermatological disorder in a subject, said method comprising administering Compound I-V and/or its isomer, polymorph, pharmaceutically acceptable salt, N-oxide, hydrate or any combination thereof and/or a pharmaceutical composition comprising the same as herein described. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the method comprises administering to a subject a pharmaceutical composition comprising Compound I-V and anti-cancer agent, an immunomodulating agent, an agent treating a dermatological disorder, an anti-infective agent, a gene therapy agent, an agent treating the endocrine system, vitamins, or a combination thereof. In some embodiments, dermatological disorders comprise, but are not limited to, acne, actinic keratosis, alopecia, androgenic alopecia, alopecia areata, alopecia secondary to chemotherapy, alopecia secondary to radiation therapy, alopecia induced by scarring, alopecia induced by stress, angioma, atopic dermatitis, baldness, ichthyosis, impetigo, lichen planus, lichen simplex chronicus, lipoma, lymphadenitis, malignant melanoma, melasma, miliaria, molluscum contagiosum, nummular dermatitis, pemphigus, perioral dermatitis, photoallergy, photosensitivity, psoriasis, raynaud's disease, ring worm, rosacea, scabies, scleroderma, vitiligo, or warts.

In one embodiment, the dermatological disorder is a wound or a burn. In some embodiments, wounds and/or ulcers are found protruding from the skin or on a mucosal surface or as a result of an infarction in an organ. A wound may be a result of a soft tissue defect or a lesion or of an underlying condition. In one embodiment, the term “wound” denotes a bodily injury with disruption of the normal integrity of tissue structures. The term is also intended to encompass the terms “sore”, “lesion”, “necrosis” and “ulcer”. In one embodiment, the term “sore” refers to any lesion of the skin or mucous membranes and the term “ulcer” refers to a local defect, or excavation, of the surface of an organ or tissue, which is produced by the sloughing of necrotic tissue. Lesion generally relates to any tissue defect. Necrosis is related to dead tissue resulting from infection, injury, inflammation or infarctions. All of these are encompassed by the term “wound”, which denotes any wound at any particular stage in the healing process including the stage before any healing has initiated or even before a specific wound like a surgical incision is made (prophylactic treatment).

Examples of wounds which can be prevented and/or treated in accordance with the present invention are, e.g., aseptic wounds, contused wounds, incised wounds, lacerated wounds, non-penetrating wounds (i.e. wounds in which there is no disruption of the skin but there is injury to underlying structures), open wounds, penetrating wounds, perforating wounds, puncture wounds, septic wounds, subcutaneous wounds, etc. Examples of sores are bed sores, canker sores, chrome sores, cold sores, pressure sores etc. Examples of ulcers are, e.g., peptic ulcer, duodenal ulcer, gastric ulcer, gouty ulcer, diabetic ulcer, hypertensive ischemic ulcer, stasis ulcer, ulcus cruris (venous ulcer), sublingual ulcer, submucous ulcer, symptomatic ulcer, trophic ulcer, tropical ulcer, veneral ulcer, e.g. caused by gonorrhoea (including urethritis, endocervicitis and proctitis). In the present context the term “wounds” encompasses the term “ulcer”, “lesion”, “sore” and “infarction”, and the terms are indiscriminately used unless otherwise indicated.

The kinds of wounds to be treated according to the invention include also i) general wounds such as, e.g., surgical, traumatic, infectious, ischemic, thermal, chemical and bullous wounds; ii) wounds specific for the oral cavity such as, e.g., post-extraction wounds, endodontic wounds especially in connection with treatment of cysts and abscesses, ulcers and lesions of bacterial, viral or autoimmunological origin, mechanical, chemical, thermal, infectious and lichenoid wounds; herpes ulcers, stomatitis aphthosa, acute necrotising ulcerative gingivitis and burning mouth syndrome are specific examples; and iii) wounds on the skin such as, e.g., neoplasm, burns (e.g. chemical, thermal), lesions (bacterial, viral, autoimmunological), bites and surgical incisions. Another way of classifying wounds is as i) small tissue loss due to surgical incisions, minor abrasions and minor bites, or as ii) significant tissue loss. The latter group includes ischemic ulcers, pressure sores, fistulae, lacerations, severe bites, thermal burns and donor site wounds (in soft and hard tissues) and infarctions.

In other aspects of the invention, the wound to be prevented and/or treated is selected from the group consisting of aseptic wounds, infarctions, contused wounds, incised wounds, lacerated wounds, non-penetrating wounds, open wounds, penetrating wounds, perforating wounds, puncture wounds, septic wounds and subcutaneous wounds.

Other wounds which are of importance in connection with the present invention are wounds like ischemic ulcers, pressure sores, fistulae, severe bites, thermal burns and donor site wounds.

In one embodiment, the pharmaceutical composition as described herein is useful in wound healing as an adjunct to physical therapy/rehabilitation, as an anabolic agent. In another embodiment, the pharmaceutical composition as described herein is useful in promoting healing of anterior cruciate ligament (ACL) or medial cruciate ligament (MCL) injuries, or accelerating recovery after ACL or MCL surgery. In another embodiment, the pharmaceutical composition as described herein is useful in promoting muscle growth in a subject with muscle atrophy secondary to being in a post surgical state. In another embodiment, the pharmaceutical composition as described herein is useful in enhancing athletic performance. In another embodiment, the pharmaceutical composition as described herein is useful in treating bums. In another embodiment, the pharmaceutical composition as described herein is useful in stimulating cartilage regrowth. In another embodiment, the pharmaceutical composition as described herein is useful in preventing, treating, or reversing of catabolism associated with prolonged critical illness, pulmonary dysfunction, ventilator dependency, aging, AIDS, trauma, surgery, congestive heart failure, cardiac myopathy, bums, cancer, COPD. In another embodiment, the pharmaceutical composition as described herein is useful in preventing or reversing protein catabolism due to trauma. In another embodiment, the pharmaceutical composition as described herein is useful as a) adjunct to cauterization therapy (laser or radio) as is used in surgery to promote wound healing, b) adjunct to cryotherapy to promote wound healing, c) adjunct to chemotherapy to prevent side effects.

In some embodiments, bums are associated with reduced testosterone levels, and hypgonadism is associated with delayed wound healing. In one embodiment, the methods of this invention, provide for treating a subject suffering from a wound or a bum.

In some embodiments, the present invention provides a method for treating, reducing the incidence, delaying the onset or progression, or reducing and/or abrogating the symptoms associated with a kidney disease in a subject, said method comprising administering to a subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the methods of this invention are useful in subjects predisposed to kidney diseases or disorders. In one embodiment, the phrase “predisposed to a kidney disease or disorder” with respect to a subject is synonymous with the phrase “subject at risk”, and includes a subject at risk of acute or chronic renal failure, or at risk of the need for renal replacement therapy, if the subject is reasonably expected to suffer a progressive loss of renal function associated with progressive loss of functioning nephron units. Whether a particular subject is at risk is a determination which may routinely be made by one of ordinary skill in the relevant medical or veterinary art.

In some embodiments, the present invention provides a method for treating, reducing the incidence, delaying the onset or progression, or reducing and/or abrogating the symptoms associated with a connective tissue disease in a subject, said method comprising administering to said subject Compound I-V and/or its isomer, polymorph, pharmaceutically acceptable salt, N-oxide, hydrate or any combination thereof, and/or a pharmaceutical composition comprising the same as herein described. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In one embodiment, the method comprises administering to a subject a pharmaceutical composition comprising a SARM compound and/or a pharmaceutical composition comprising the same as herein described and anti-cancer agent, an immunomodulating agent, an agent treating a dermatological disorder, an anti-infective agent, an agent treating a metabolic disease, an agent treating a wasting disease, a gene therapy agent, an agent treating the endocrine system, vitamins, or a combination thereof. In some embodiments, connective tissue diseases comprise ankylosing spondylitis, Ehlers-Danlos syndrome, Henoch-Schonlein purpura, Kawasaki disease, Marfan syndrome, polyarteritis nodosa, polymyositis, psoriatic arthritis, reactive arthritis, rheumatoid arthritis, scleroderma, Sjogren's syndrome, Still's disease, systemic lupus erythematosus, Takayasu disease, or Wegener's granulomatosis.

In some embodiments, the present invention provides a method for treating, reducing the incidence, delaying the onset or progression, or reducing and/or abrogating the symptoms associated with an ophthalmic disease in a subject, said method comprising administering to a subject Compound I-V and/or its isomer, polymorph, pharmaceutically acceptable salt, N-oxide, hydrate or any combination thereof, and/or a pharmaceutical composition comprising the same as herein described. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments ophthalmic disease comprises, but is not limited to, acute zonal occult outer retinopathy, Adie syndrome, albinism, ocular-amaurosis, fugax, amblyopia, blepharitis, blepharoptosis, blepharospasm, cataract, chalazion, conjunctivitis, corneal diseases, corneal dystrophies, corneal edema, corneal ulcer, diabetic retinopathy, dry eye syndromes, Duane retraction syndrome, ectropion, entropion, esotropia, exfoliation syndrome, exotropia, eye hemorrhage, eye neoplasms, eyelid diseases, floaters, general fibrosis syndrome, glaucoma, gyrate atrophy, hemianopsia, Hermanski-Pudlak syndrome, hordeolum, Homer syndrome, iritis, Kearns-Sayer syndrome, keratitis, keratoconus, lacrimal apparatus diseases, lacrimal duct obstruction, lens diseases, macular degeneration, microphthalmos, myopia, nystagmus, pathologic, ocular motility disorders, oculomotor nerve diseases, ophthalmoplegia, optic atrophies, optic nerve diseases, optic neuritis, optic neuropathy, orbital cellulitis, papilledema, retinal detachment, retinal diseases, retinal vein occlusion, retinitis pigmentosa, retinopathy of prematurity, retinoschisis, scleritis, scotoma, Thygeson's superficial punctate keratitis, trachoma, uveitis, white dot syndrome, vision disorders, or vitreous disorders.

In some embodiments, the present invention provides a method for prevention of statin induced rhabdomyolysis. In some embodiments, the present invention provides a method for prevention of statin induced rhabdomyolysis, organ failure or insufficiency.

In some embodiments, the present invention provides a method for treating, reducing the incidence, delaying the onset or progression, or reducing and/or abrogating the symptoms associated with a stomatognathic disease in a subject, said method comprising administering to said subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the method comprises administering to a subject a pharmaceutical composition comprising compound I-V and/or a pharmaceutical composition comprising the same as herein described and an anti-cancer agent, an immunomodulating agent, an anti-infective agent, an agent treating a wasting disease, a gene therapy agent, an agent treating the endocrine system, vitamins, or a combination thereof. In some embodiments, stomatognathic diseases comprise ankyloglossia, bruxism, burning mouth syndrome, cheilitis, cherubism, cleft lip, dentigerous cyst, gingivitis, glossitis, benign migratory, herpes labialis, Ludwig's angina, macroglossia, Melkersson-Rosenthal syndrome, periodontal diseases, Pierre Robin syndrome, prognathism, salivary gland diseases, sialorrhea, stomatitis, aphthous, temporomandibular joint disorders, temporomandibular joint dysfunction syndrome, or xerostomia.

In some embodiments, the present invention provides a method for treating, reducing the incidence, delaying the onset or progression, or reducing and/or abrogating the symptoms associated with a respiratory tract disease in a subject, said method comprising administering to a subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the method comprises administering to a subject Compound I-V and/or a pharmaceutical composition comprising the same as herein described and an anti-cancer agent, an immunomodulating agent, an agent treating the central nervous system, an agent treating the cardiovascular system, an anti-infective agent, an agent treating a wasting disease, a gene therapy agent, an agent treating the endocrine system, vitamins, or a combination thereof. In some embodiments, respiratory tract diseases comprise airway obstruction, apnea, asbestosis, asthma, atelectasis, berylliosis, bronchial diseases, bronchiectasis, bronchiolitis, bronchiolitis obliterans organizing pneumonia, bronchitis, bronchopulmonary dysplasia, common cold, cough, empyema, pleural, epiglottitis, hemoptysis, hypertension, pulmonary, hyperventilation, Kartagener syndrome, lung abscess, lung diseases, meconium aspiration syndrome, pleural effusion, pleurisy, pneumonia, pneumothorax, pulmonary alveolar proteinosis, pulmonary disease, chronic obstructive, pulmonary edema, pulmonary embolism, pulmonary emphysema, pulmonary fibrosis, respiratory distress syndrome, newborn-respiratory hypersensitivity, respiratory tract infections, rhinoscleroma, scimitar syndrome, severe acute respiratory syndrome, silicosis, sleep apnea, central stridor, tracheal stenosis, Wegener's granulomatosis, or whooping cough.

In some embodiments, the present invention provides a method for treating, reducing the incidence, delaying the onset or progression, or reducing and/or abrogating the symptoms associated with an otorhinolaryngologic disease in a subject, said method comprising administering to the subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the method comprises administering to a subject a pharmaceutical composition comprising Compound I-V and an anti-cancer agent, an immunomodulating agent, an anti-infective agent, an agent treating a wasting disease, a gene therapy agent, an agent treating the endocrine system, vitamins, or a combination thereof. In some embodiments, otorhinolaryngologic diseases comprise cholesteatoma, middle ear, croup, deafness, epistaxis, hearing loss, hyperacusis, labyrinthitis, laryngitis, laryngomalacia, laryngostenosis, mastoiditis, Meniere's disease, nasal obstruction, nasal polyps, otitis, otorhinolaryngologic diseases, otosclerosis, pharyngitis, presbycusis, retropharyngeal abscess, rhinitis, sinusitis, tinnitus, tonsillitis, tympanic membrane perforation, vestibular neuronitis, vocal cord paralysis, or voice disorders.

In one embodiment, a wide variety of injuries of the CNS may be treated by the methods of the present invention. CNS injury may refer, in one embodiment, to a breakdown of the membrane of a nerve cell, or, in another embodiment, to the inability of the nerve to produce and propagate nerve impulses, or in another embodiment, to the death of the cell. An injury includes damage that directly or indirectly affects the normal functioning of the CNS. The injury may be a structural, physical, or mechanical impairment and may be caused by physical impact, as in the case of a crushing, compression, or stretching of nerve fibers. Alternatively, the cell membrane may be destroyed by or degraded by an illness, a chemical imbalance, or a physiological malfunction such as anoxia (e.g., stroke), aneurysm, or reperfusion. A CNS injury includes, for example and without limitation, damage to retinal ganglion cells, a traumatic brain injury, a stroke-related injury, a cerebral aneurism-related injury, a spinal cord injury, including monoplegia, diplegia, paraplegia, hemiplegia and quadriplegia, a neuroproliferative disorder, or neuropathic pain syndrome.

In some embodiments, central nervous system diseases comprise injuries or damage to the central nervous system (CNS). In some embodiments, injuries or damage to the CNS may be associated with muscle wasting disorders, central nerve injury or damage, peripheral nerve injury or damage and spinal cord injury or damage.

Injuries to the spinal cord may arise from compression or other contusion of the spinal cord, or a crushing or severing of the spinal cord. A severing of the spinal cord, also referred to herein as a “transection,” may be a complete severing or, may be an incomplete severing of the spinal cord.

In some embodiments, the methods of treating a subject suffering from a CNS injury or, in other embodiments, spinal cord injury, may be accompanied by treatment of the subject with electrical stimulation of the injured site and the administration of a purine nucleoside, or analog thereof, for example as described in United States Patent Application Publication Number 20040214790A1.

In some embodiments, the present invention provides a method for treating, reducing the incidence, delaying the onset or progression, or reducing and/or abrogating the symptoms associated with an urologic and/or male genital disease in a subject, said method comprising administering to the subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the method comprises administering to a subject a pharmaceutical composition comprising Compound I-V and a second therapeutic compound, as herein described, sutiable for such treatment, as will be appreciated by the skilled artisan.

In some embodiments, the present invention provides a method for treating, reducing the incidence, delaying the onset or progression, or reducing and/or abrogating the symptoms associated with urogenital disease and/or fertility in a subject, said method comprising administering to the subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In one embodiment, the method comprises administering to a subject a pharmaceutical composition comprising a Compound I-V and and a second therapeutic compound, as herein described, sutiable for such treatment, as will be appreciated by the skilled artisan.

In some embodiments, the present invention provides a method for treating, reducing the incidence, delaying the onset or progression, or reducing and/or abrogating the symptoms associated with hemic and/or lymphatic disease in a subject, said method comprising administering to the subject Compound I-V and/or a pharmaceutical composition comprising the same as herein described and/or its isomer,polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I.

In some embodiments, the present invention provides a method for treating, reducing the incidence, delaying the onset or progression, or reducing and/or abrogating the symptoms associated with a congenital, hereditary, or neonatal disease in a subject, said method comprising administering to the subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In some embodiments, congenital, hereditary, and neonatal diseases comprise, but is not limited to, Aicardi syndrome, amniotic band syndrome, anencephaly, branchio-oto-renal syndrome, cat eye syndrome, cerebral gigantism-charge syndrome, chromosome 16 abnormalities, chromosome 18 abnormalities, chromosome 20 abnormalities, chromosome 22 abnormalities, Costello syndrome, cri-du-chat syndrome, cystic fibrosis, de-Lange syndrome, distal trisomy 10q, down syndrome, ectodermal dysplasia, fetal alcohol syndrome, fetal diseases, fragile x syndrome, Freeman-Sheldon syndrome, jaundice, Klinefelter syndrome, monosomy 9p, nail-patella syndrome, neurofibromatoses, neuronal ceroid-lipofuscinosis, ochoa syndrome (urofacial syndrome, hydronephrosis with peculiar facial expression), oculocerebrorenal syndrome, Pallister-Killian syndrome, Prader-Willi syndrome, proteus syndrome, prune belly syndrome, Rett syndrome, Robinow syndrome, Rubinstein-Taybi syndrome, schizencephaly, situs inversus, Smith-Lemli-Opitz syndrome, congenital, trichothiodystrophy, triple-x females, trisomy 13 (Patau syndrome), trisomy 9, turner syndrome, twins, conjoined, Usher syndrome, Waardenburg's syndrome, Werner syndrome, or Wolf-Hirschhorn syndrome.

In some embodiments, the present invention provides a method for enhanced production such as milk, sperm, or egg, said method comprising administering to a subject Compound I-V and/or a pharmaceutical composition comprising the same as herein described and/or its isomer, polymorph, pharmaceutically acceptable salt, N-oxide, hydrate or any combination thereof. In another embodiment, the pharmaceutical composition comprises Compound S-I. In another embodiment the composition is a capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I. In another embodiment, the capsule comprises Compound S-I. In another embodiment the pharmaceutical composition is a tablet comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the tablet consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the tablet comprises Compound S-I. In another embodiment the composition is a softgel capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the softgel capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the composition is a powder-filled capsule comprising 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment the powder-filled capsule consisting essentially of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound I-V. In another embodiment, the softgel capsule comprises Compound S-I. In another embodiment the softgel capsule consisting essentially of 3 mg of Compound S-I. In some embodiments, the present invention provides a method for enhanced production of lean meats or eggs. In some embodiments, the present invention provides a method for increased productivity of feeds or stud livestock, for example, increased sperm count, improved morphology of sperm, etc. In some embodiments, the present invention provides a method for expanding the productive life of farm animals, for example, egg-laying hens, milk-producing cows, etc, and/or enhanced herd health, for example, improved immune clearance, stronger animals.

In another embodiment, this invention provides methods of treatment of cystic fibrosis and induced hypogonadal states as a result of the same, epilepsy and induced hypogonadal and/or hypermetabolic states as a result of the same, hereditary angioedema, lupus erythematosus and decreased BMD as a result of the same, alcohol and smoking induced osteoporosis, in a subject the methods comprising administering a pharmaceutical composition as herein described to the subject.

In another embodiment, this invention provides a method of treating Opioid Induced Androgen Deficiency (OPIAD), the method comprising administering to the subject a SARM as herein described, and optionally opiates, opioids, narcotics, used in treatment of heroin addiction, opiates/opioids used in the treatment of chronic pain of malignancy, opiates/opioids used in the treatment non-malignant of chronic pain syndromes.

In another embodiment, the pharmaceutical composition as described herein is useful in promoting or speeding recovery following a surgical procedure.

In some embodiments, the present invention provides a method for treating, reducing the incidence, delaying the onset or progression, or reducing and/or abrogating the symptoms associated with a combination of diseases and/or disorders in a subject as described hereinabove.

It is to be understood that any method of this invention, as herein described, encompasses the administration of any pharmaceutical composition comprising Compound I-V to the subject, in order to treat the indicated disease, disorder or condition. The methods as herein described each and/or all may further comprise administration of an additional therapeutic agent as herein described, and as will be appreciated by one skilled in the art.

It is to be understood that any use of any of the pharmaceutical composition as herein described in the treatment of any disease, disorder or condition as described herein, is to be considered an embodiment of this invention.

The following examples are presented in order to more fully illustrate the preferred embodiments of the invention. They should in no way be construed, however, as limiting the broad scope of the invention.

### Experimental Details Section

### EXAMPLE 1

### Synthesis of Compound S-I

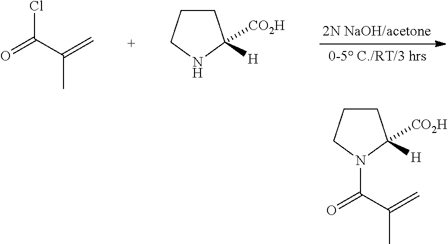

Step 1: (2R)-1-Methacryloylpyrrolidin-2-carboxylic Acid. D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, room temperature (rt)), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) 8 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral canter), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C=0), 1647 (CO, COOH), 1584, 1508, 1459, 1369, 1348, 1178 cm⁻¹; [α]_{D}²⁶ +8° (c=1, MeOH); Anal. Calcd. for C₉H₁₃NO₃: C 59.00, H 7.15, N 7.65. Found: C 59.13, H 7.19, N 7.61.

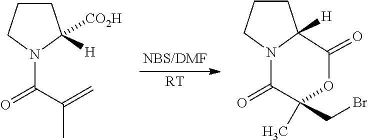

Step 2: (3R,8aR)-3-Bromomethyl-3-methyl-tetrahydro-pyrrolo[2,1-c][1,4]oxazine-1,4-dione. A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at rt, and the resulting mixture was stirred 3 days. The solvent was removed in vacuo, and a yellow solid was precipitated. The solid was suspended in water, stirred overnight at rt, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062cm⁻¹; [α]_{D}²⁶ +124.5° (c=1.3, chloroform); Anal. Calcd. for C₉H₁₂BrNO₃: C 41.24, H 4.61, N 5.34. Found: C 41.46, H 4.64, N 5.32.

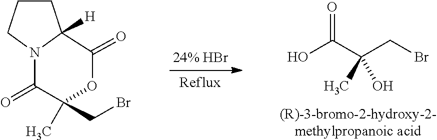

Step 3: (2R)-3-Bromo-2-hydroxy-2-methylpropanoic Acid. A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite, and evaporated in vacuo to dryness. Recrystallization from toluene afforded 10.2 g (86%) of the desired compound as colorless crystals: mp 107-109° C.; ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶ +10.5° (c=2.6, MeOH); Anal. Calcd. for C₄H₇BrO₃: C 26.25, H 3.86. Found: C 26.28, H 3.75.

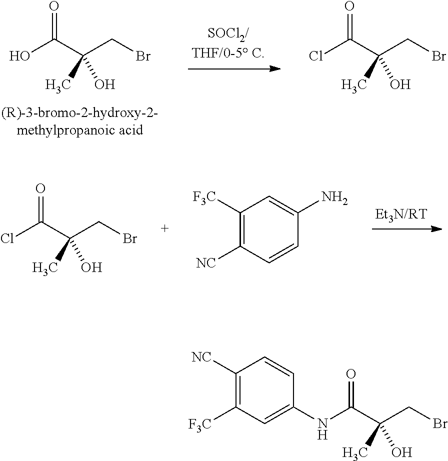

Step 4: Synthesis of (2R)-3-bromo-N-[4-cyano-3-(trifluoromethyl)phenyl]-2-hydroxy-2-methylpropanamide. Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at rt. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid. ¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M−H]⁻ 349.0. M.p.: 124-126° C.

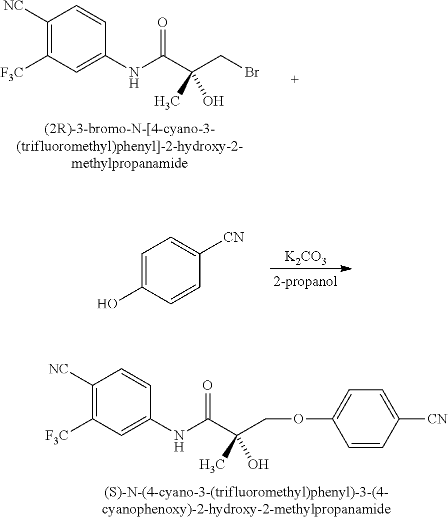

Step 5: Synthesis of (S)—N-(4-cyano-3-(trifluoromethyl)phenyl)-3-(4-cyanophenoxy)-2-hydroxy-2-methylpropanamide (S-I). A mixture of bromoamide ((2R)-3-bromo-N-[4-cyano-3-(trifluoromethyl)phenyl]-2-hydroxy-2-methylpropanamide, 50 g, 0.14 mol), anhydrous K₂CO₃ (59.04 g, 0.43 mol), 4-cyanophenol (25.44 g, 0.21 mol) in 500 mL of 2-propanol was heated to reflux for 3 h and then concentrated under reduced pressure to give a solid. The resulting residue was treated with 500 mL of H₂O and then extracted with EtOAc (2×300 mL). The combined EtOAc extracts were washed with 10% NaOH (4×200 mL) and brine. The organic layer was dried over MgSO₄ and then concentrated under reduced pressure to give an oil which was treated with 300 mL of ethanol and an activated carbon. The reaction mixture was heated to reflux for 1 h and then the hot mixture was filtered through Celite. The filtrate was concentrated under reduced pressure to give an oil. This oil was purified by column chromatography using CH₂Cl₂/EtOAc (80:20) to give an oil which was crystallized from CH₂Cl₂/hexane to give 33.2 g (59.9%) of (S)—N-(4-cyano-3-(trifluoromethyl)phenyl)-3-(4-cyanophenoxy)-2-hydroxy-2-methylpropanamide as a colorless solid (a cotton type). ¹H NMR (CDCl₃/TMS) δ 1.63 (s, 3H, CH₃), 3.35 (s, 1H₂OH), 4.07 (d, J=9.04 Hz, 1H, CH), 4.51 (d, J=9.04 Hz, 1H, CH), 6.97-6.99 (m, 2H, ArH), 7.57-7.60 (m, 2H, ArH), 7.81 (d, J=8.55 Hz, 1H, ArH), 7.97 (dd, J=1.95, 8.55 Hz, 1H, ArH), 8.12 (d, J=1.95 Hz, 1H, ArH), 9.13 (bs, 1H, NH). Calculated Mass: 389.10, [M−H]⁻ 388.1. Mp: 92-94° C.

Thus Compound S-I was synthesized in one embodiment, according to the method hereinabove.

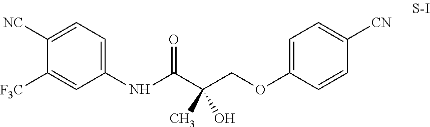 [SMILES: C[C@](O)(COc1ccc(C#N)cc1)C(=O)Nc1ccc(C#N)c(C(F)(F)F)c1]

### Synthesis of Compound S-II

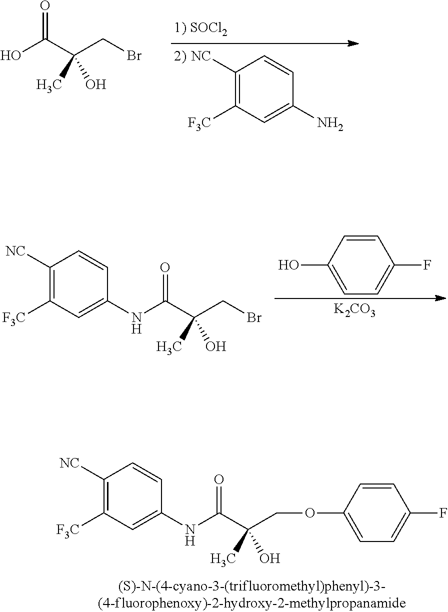

### Step 1: Synthesis of (2R)-3-bromo-N-[4-cyano-3-(trifluoromethyl)phenyl]-2-hydroxy-2-methylpropanamide

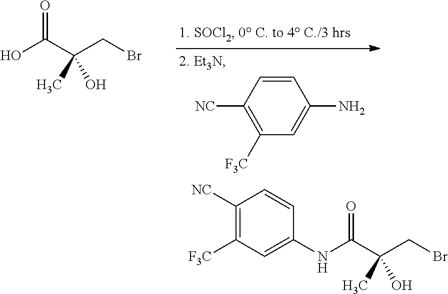

Thionyl chloride (46.02 g, 0.39 mol) was added dropwise to a cooled solution of bromoacid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at rt. The solvent was removed under reduced pressure to give a solid which was treated with 300 mL of H₂O, extracted with EtOAc (2×400 mL). The combined organic extracts were washed with a saturated NaHCO₃ solution (2×300 mL) and brine (300 mL). The organic layer was dried over MgSO₄ and concentrated under reduced pressure to give a solid which was purified by column chromatography using CH₂Cl₂/EtOAc (80:20 v/v) to give a solid. This solid was recrystallized from CH₂Cl₂/hexane to provide 55.8 g of the bromoamide compound (73.9%) as a light-yellow solid. ¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, MS (ESI) m/z 349.0 [M−H]⁻. M.p.: 124-126° C.

### Step 2: Synthesis of (S)—N-(4-cyano-3-(trifluoromethyl)phenyl)-3-(4-fluorophenoxy)-2-hydroxy-2-methylpropanamide

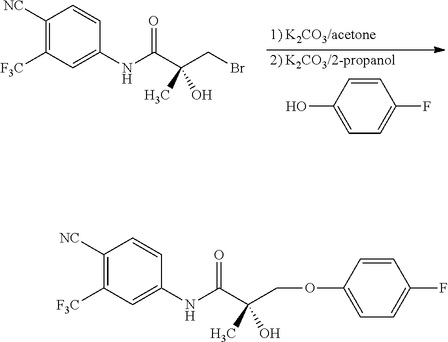

A mixture of bromoamide (18.84 g, 53.66 mmol), anhydrous K₂CO₃ (22.25 g, 160.97 mmol) in 300 mL of acetone was heated to reflux for 1 h and then concentrated under reduced pressure to give a solid. The resulting residue was treated with 4-fluorophenol (9.02 g, 80.49 mmol), anhydrous K₂CO₃ (14.83 g, 107.31 mmol), 300 mL of 2-propanol and then heated to reflux for 2 h. The resulting mixture was concentrated under reduced pressure to give a solid. This solid was treated with 300 mL of H₂O and extracted with EtOAc (2×250 mL). The combined EtOAc extracts were washed with a saturated NaHCO₃ solution (2×250 mL) and brine. The organic layer was dried over MgSO₄ and then concentrated under reduced pressure to give an oil which was purified by column chromatography using CH₂Cl₂/EtOAc (80:20) to give a solid which was recrystallized from CH₂Cl₂/hexane to provide 16.52 g of (S)—N-(4-cyano-3-(trifluoromethyl)phenyl)-3-(4-fluorophenoxy)-2-hydroxy-2-methylpropanamide (80.5%) as a white solid.

¹H NMR (CDCl₃/TMS) δ 1.59 (s, 3H, CH₃), 3.36 (s, 1H₂OH), 3.95 (d, J=9.00 Hz, 1H, CH), 4.43 (d, J=9.00 Hz, 1H, CH), 6.87-6.88 (m, 2H, ArH), 6.96-7.02 (m, 2H, ArH), 7.81 (d, J=8.45 Hz, 1H, ArH), 7.94-7.98 (m, 1H, ArH), 8.10 (d, J=1.79 Hz, 1H, ArH), 9.11 (bs, 1H, NH). Calculated Mass: 382.09, MS (ESI) m/z 380.9 [M−H]⁻. M.p.: 139-140° C.

### Synthesis of (S) Enantiomer of Compound of Formula III (S-III)

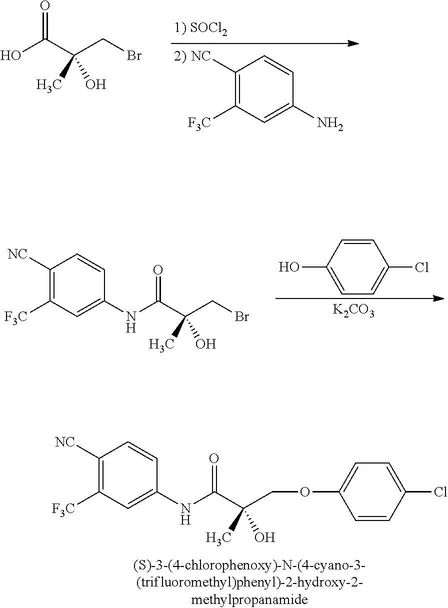

Step 1: Synthesis of (2R)-3-bromo-N-[4-cyano-3-(trifluoromethyl)phenyl]-2-hydroxy-2-methylpropanamide

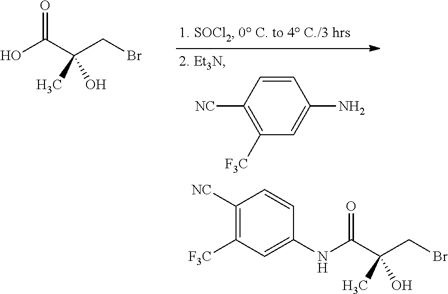

Thionyl chloride (46.02 g, 0.39 mol) was added dropwise to a cooled solution of bromoacid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at rt. The solvent was removed under reduced pressure to give a solid which was treated with 300 mL of H₂O, extracted with EtOAc (2×400 mL). The combined organic extracts were washed with a saturated NaHCO₃ solution (2×300 mL) and brine (300 mL). The organic layer was dried over MgSO₄ and concentrated under reduced pressure to give a solid which was purified by column chromatography using CH₂Cl₂/EtOAc (80:20 v/v) to give a solid. This solid was recrystallized from CH₂Cl₂/hexane to provide 55.8 g of the bromoamide compound (73.9%) as a light-yellow solid. ¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, MS (ESI) m/z 349.0 [M−H]⁻. M.p.: 124-126° C.

### Step 2: Synthesis of (S)-3-(4-chlorophenoxy)-N-(4-cyano-3-(trifluoromethyl)phenyl)-2-hydroxy-2-methylpropanamide

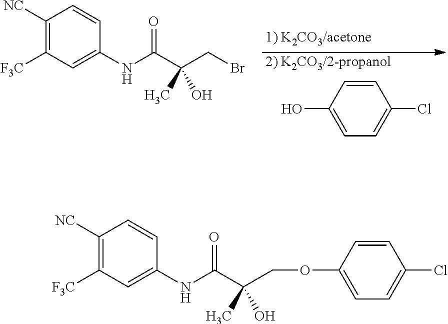

A mixture of bromoamide (13.0 g, 37.02 mmol), anhydrous K₂CO₃ (15.35 g, 111.07 mmol) in 200 mL of acetone was heated to reflux for 1 h and then concentrated under reduced pressure to give a solid. The resulting residue was treated with 4-chlorophenol (7.14 g, 55.54 mmol), anhydrous K₂CO₃ (10.23 g, 74.05 mmol), 200 mL of 2-propanol and then heated to reflux for 2 h. The resulting mixture was concentrated under reduced pressure to give a solid. This solid was treated with 200 mL of H₂O and extracted with EtOAc (2×100 mL). The combined EtOAc extracts were washed with a saturated NaHCO₃ solution (2×100 mL) and brine. The organic layer was dried over MgSO₄ and then concentrated under reduced pressure to give a solid which was purified by column chromatography using CH₂Cl₂/EtOAc (80:20 v/v) to give a solid. This solid was recrystallized from CH₂Cl₂/hexane to provide 8.96 g of (S)-3-(4-chlorophenoxy)-N-(4-cyano-3-(trifluoromethyl)phenyl)-2-hydroxy-2-methylpropanamide (60.7%) as a white solid. ¹H NMR (CDCl₃/TMS) δ 1.59 (s, 3H, CH₃), 3.37 (s, 1H₂OH), 3.97 (d, J=9.03 Hz, 1H, CH), 4.44 (d, J=9.03 Hz, 1H, CH), 6.83-6.86 (m, 2H, ArH), 7.23-7.26 (m, 2H, ArH), 7.80 (d, J=8.54 Hz, 1H, ArH), 7.96 (dd, J=1.95, 8.54 Hz, 1H, ArH), 8.11 (d, J=1.95 Hz, 1H, ArH), 9.12 (bs, 1H, NH). Calculated Mass: 398.06, MS (ESI) m/z 396.9 [M−H]⁻. M.p.: 146-148° C.

### Synthesis of (S) Enantiomer of Compound of Formula IV (S-IV)

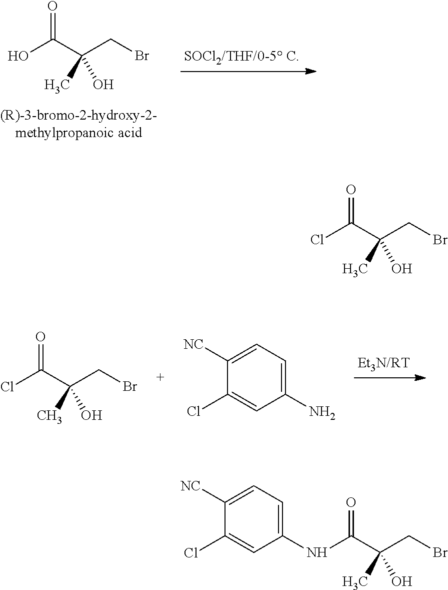

Step 1: Synthesis of (2R)-3-bromo-N-(3-chloro-4-cyanophenyl)-2-hydroxy-2-methylpropanamide. Thionyl chloride (7.8 g, 65 5 mmol) was added dropwise to a cooled solution (less than 4° C.) of (R)-3-bromo-2-hydroxy-2-methylpropanoic acid (9.0 g, 49.2 mol) in 50 mL of THF under an argon atmosphere. The resulting mixture was stirred for 3 h under the same condition. To this was added Et₃N (6.6 g, 65.5 mol) and stirred for 20 min under the same condition. After 20 min, 4-amino-2-chlorobenzonitrile (5.0 g, 32 8 mmol) and 100 mL of THF were added and then the mixture was allowed to stir overnight at rt. The solvent was removed under reduced pressure to give a solid which was treated with 100 mL of H₂O, extracted with EtOAc (2×150 mL). The combined organic extracts were washed with saturated NaHCO₃ solution (2×100 mL) and brine (300 mL), successively. The organic layer was dried over MgSO₄ and concentrated under reduced pressure to give a solid which was purified from column chromatography using EtOAc/hexane (50:50) to give 7.7 g (49.4%) of target compound as a brown solid.

¹H NMR (CDCl₃/TMS) δ 1.7 (s, 3H, CH₃), 3.0 (s, 1H, OH), 3.7 (d, 1H, CH), 4.0 (d, 1H, CH), 7.5 (d, 1H, ArH), 7.7 (d, 1H, ArH), 8.0 (s, 1H, ArH), 8.8 (s, 1H, NH). MS:342.1 (M+23). Mp 129° C.

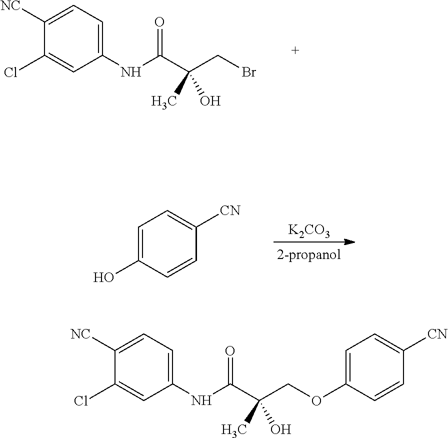

Step 2: Synthesis of (S)—N-(3-chloro-4-cyanophenyl)-3-(4-cyanophenoxy)-2-hydroxy-2-methylpropanamide ((S)-isomer of compound of formula IV (S-IV)). A mixture of bromoamide (2.0 g, 6.3 mmol), anhydrous K₂CO₃ (2.6 g, 18 9 mmol) in 50 mL of acetone was heated to reflux for 2 h and then concentrated under reduced pressure to give a solid. The resulting solid was treated with 4-cyanophenol (1.1 g, 9 5 mmol) and anhydrous K₂CO₃ (1.7 g, 12 6 mmol) in 50 mL of 2-propanol was heated to reflux for 3 h and then concentrated under reduced pressure to give a solid. The residue was treated with 100 mL of H₂O and then extracted with EtOAc (2×100 mL). The combined EtOAc extracts were washed with 10% NaOH (4×100 mL) and brine, successively. The organic layer was dried over MgSO₄ and then concentrated under reduced pressure to give an oil which was purified by column chromatography using EtOAc/hexane (50:50) to give a solid. The solid was recrystallized from CH₂Cl₂/hexane to give 1.4 g (61.6%) of (S)—N-(3-chloro-4-cyanophenyl)-3-(4-cyanophenoxy)-2-hydroxy-2-methylpropanamide as a colorless solid.

¹H NMR (CDCl₃/TMS) δ 1.61 (s, 3H, CH₃), 3.25 (s, 1H₂OH), 4.06 (d, J=9.15 Hz, 1H, CH), 4.50 (d, J=9.15 Hz, 1H, CH), 6.97-6.99 (m, 2H, ArH), 7.53-7.59 (m, 4H, ArH), 7.97 (d, J=2.01 Hz, 1H, ArH), 8.96 (s, 1H, NH). Calculated Mass: 355.1, [M+Na]⁺ 378.0. Mp: 103-105° C.

### Synthesis of (S) Enantiomer of Compound of Formula V (S-V)

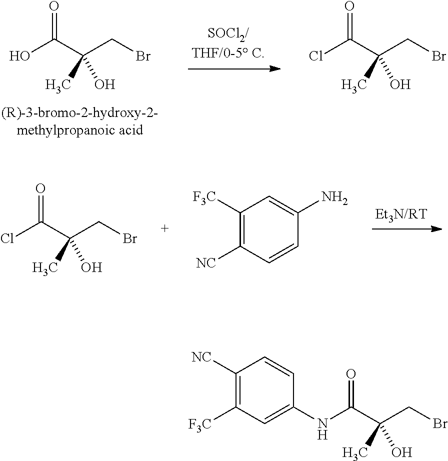

Step 1: Synthesis of (2R)-3-Bromo-N[4-cyano-3-(trifluoromethyl)phenyl]-2-hydroxy-2-methylpropanamide. Thionyl chloride (46.02 g, 0.39 mol) was added dropwise to a cooled solution (less than 4° C.) of R-18 (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at rt. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide (R-18) as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M−H]⁻ 349.0. M.p.: 124-126° C.

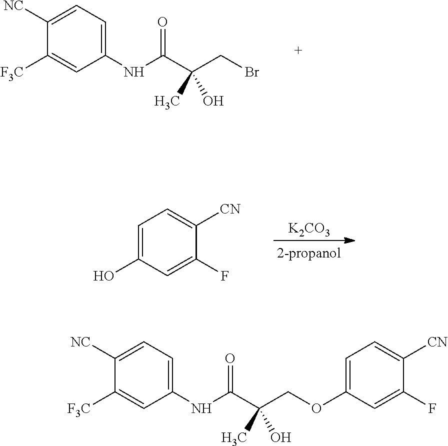

Step 2: Synthesis of (S)—N-(4-cyano-3-(trifluoromethyl)phenyl)-3-(4-cyano-3-fluorophenoxy)-2-hydroxy-2-methylpropanamide ((S)-isomer of compound of formula V (S-V)). A mixture of bromoamide ((2R)-3-bromo-N-[4-cyano-3-(trifluoromethyl)phenyl]-2-hydroxy-2-methylpropanamide (2.0 g, 5.70 mmol), anhydrous K₂CO₃ (2.4 g, 17 1 mmol) in 50 mL of acetone was heated to reflux for 2 h and then concentrated under reduced pressure to give a solid. The resulting solid was treated with 2-fluoro-4-hydroxybenzonitrile (1.2 g, 8.5 mmol) and anhydrous K₂CO₃ (1.6 g, 11 4 mmol) in 50 mL of 2-propanol was heated to reflux for 3 h and then concentrated under reduced pressure to give a solid. The residue was treated with 100 mL of H₂O and then extracted with EtOAc (2×100 mL). The combined EtOAc extracts were washed with 10% NaOH (4×100 mL) and brine, successively. The organic layer was dried over MgSO₄ and then concentrated under reduced pressure to give an oil which was crystallized from CH₂Cl₂/hexane to give 0.5 g (23%) of (S)—N-(4-cyano-3-(trifluoromethyl)phenyl)-3-(4-cyano-3-fluorophenoxy)-2-hydroxy-2-methylpropanamide as a colorless solid.

¹H NMR (CDCl₃/TMS) δ 1.63 (s, 3H, CH₃), 3.34 (bs, 1H₂OH), 4.08 (d, J=9.17 Hz, 1H, CH), 4.50 (d, J=9.17 Hz, 1H, CH), 6.74-6.82 (m, 2H, ArH), 7.50-7.55 (m, 1H, ArH), 7.81 (d, J=8.50 Hz, 1H, ArH), 7.97 (q, J=2.03, 8.50 Hz, 1H, ArH), 8.11 (d, J=2.03 Hz, 1H, ArH), 9.12 (s, 1H, NH). Calculated Mass: 407.1, [M+Na]⁺ 430.0. Mp: 124-125° C.

### EXAMPLE 2

### Crystal forms of S-I Drug Substance

Five polymorphs were identified for API S-I as discussed in U.S. Pat. Nos. 7,977,386 and 7,968,603; and U.S. Ser. No. 13/153,427 which are all incorporated by reference. These polymorphs were:

Form A: an anhydrous crystalline form, which was produced from anhydrous solvents as well as the API manufacturing process;

Form B′: a hydrated para-crystalline form, which was produced from exposure of form A or form B″ to high humidity and temperature (i.e., above 40° C. and 75% RH).

Form B″: an anhydrous para-crystalline form, which was produced by melting either the A or B′ form and rapidly cooling;

Form C: a second anhydrous crystal form, which was produced only by evaporation of a solution of THF; and

Form D: a third anhydrous crystalline form, which was produced from anhydrous solvents under temperature controlled crystallization. Thermal analysis shows Form D as the most thermodynamically stable form (melting point=135° C. by DSC). To date, GMP drug substance manufacturing has produced exclusively polymorph form A.

Drug substance stability studies at 40° C/75% RH demonstrated a conversion from the A form to the B′ form. Drug substance that had undergone conversion from the A form to the B′ form during stability studies failed appearance and moisture testing without increased impurity formation.

Drug substance stability studies at 40° C/75% RH demonstrated a conversion from the A form to the D form. Drug substance that had undergone conversion from the A form to the D form during stability studies showed a change of the DSC endotherm from 75.31° C. to 133.2° C. without increased impurity formation.

No changes in drug substance crystal form were observed at conditions 5° C., 25° C./60% RH or 30° C./75% RH for any of the stability studies.

Drug substance was stored at not more than 25° C. and protected from moisture.

S-I 3 mg powder-filled capsules (manufactured from lot 1391-1391-04-501) were packaged in amber glass bottles with desiccant packs to protect from moisture and stored at 15-25° C. Drug substance used in the manufacture of S-I softgels, 3 mg (manufactured from lot 0802GT401) was dissolved in Polyethylene Glycol 400 (PEG-400) prior to encapsulation.

### EXAMPLE 3

### Pharmaceutical Compositions

### Powder-Filled Capsules Comprising S-I, 0.1 mg and 0.3 mg

Powder blend capsule formulations for Compound S-I, 0.1 mg were prepared using three approaches: (1) high shear dry blend and fill into size three capsules; (2) high sheer wet granulation/excipient dilution and fill into size 3 capsules; (3) direct blend and fill into size three capsules. Capsule formulations consisted of micronized Compound S-I, pregelatinized starch, NF (Starch 1500), lactose monohydrate, NF (Foremost 316 Fast-Flo), microcrystalline cellulose, NF (Avicel PH 102), sodium lauryl sulfate, NF (Spectrum), colloidal silicon dioxide, NF (Cab-O-Sil M5P), magnesium stearate, NF (Non-Bovine Hyqual®), white opaque NP capsule (Size 3 Capsugel), and PVP K-30, NF (Spectrum).

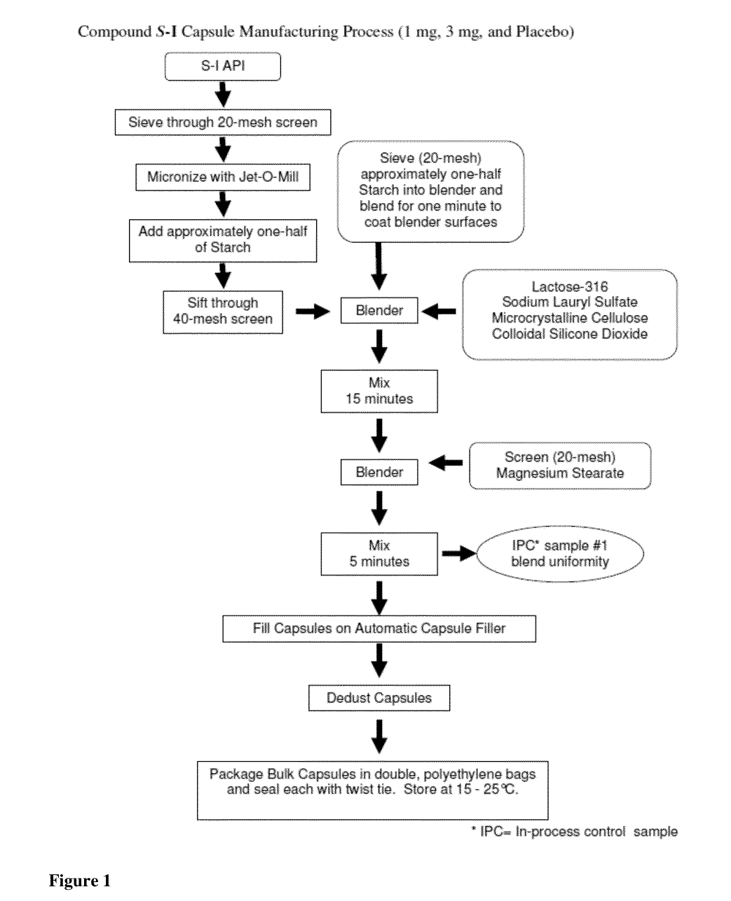

### Micronization (as Depicted in FIG. 1):

Compound S-I was micronized using a Jet-O-Mill Micronizer.

Compound S-I was passed through micronizer twice.

Nozzle Settings: Pushing 60 psi/Grinding 60 psi

Particle size was determined using Malvern Mastersizer S.

Particle sizes before and after Compound S-I micronization are:

| Particle size before | Particle size after |
|---|---|
| micronization | micronization |
| D(v, 0.1)-1.51 μm | D(v, 0.1)-0.76 μm |
| D(v, 0.5)-19.39 μm | D(v, 0.5)-8.43 μm |
| D(v, 0.9)-122.10 μm | D(v, 0.9)-52.81 μm |

In an attempt to evenly distribute the active pharmaceutical ingredient (API), a geometric blend was performed for each of the three experiments. Micronized Compound S-I was combined with a fine powder, Starch 1500 (particle size 30-150 μm, median diameter 52 μm/specific surface area 0.26 m²/g). Of the three diluents in these formulations (lactose monohydrate, microcrystalline cellulose, and starch) the one most agreeable in terms of particle size and surface area was Starch 1500.

### A. High Shear Dry Blend Process

TABLE 1
Quantitative Pharmaceutical Composition for High Shear Dry Blend Process

| | Target Weight/ | Weight/ | | Particle Size |
|---|---|---|---|---|
| Ingredients | 180 mg (mg) | Batch (g) | Function | Distribution (μm) |
| Compound S-I | 0.100 | 0.5500 | Active | ranging from |
| (Micronized) | | | | 1-70 μm |
| | | | | in length.** |
| Pregelatinized | 8.000 | 44.00 | Diluent | 30-150 μm^{φ} |
| Starch, NF | | | | |
| (Starch 1500) | | | | |
| Lactose Monohydrate, NF | 93.30 | 513.1 | Diluent | 75-250 μm^{φ} |
| (Foremost 316 Fast-Flo) | | | | |
| Microcrystalline | 75.00 | 412.5 | Diluent | 60-200 μm^{φ} |
| Cellulose, NF | | | | |
| (Avicel ® PH 102) | | | | |
| Sodium Lauryl | 1.800 | 9.900 | Surfactant | |
| Sulfate, NF | | | | |
| Colloidal Silicon | 0.900 | 4.950 | Glidant | |
| Dioxide, NF | | | | |
| (Cab-O-Sil ® M5P) | | | | |
| Magnesium Stearate, NF | 0.900 | 4.950 | Lubricant | |
| (Non-bovine Hyqual ®) | | | | |
| TOTAL | 180.0 | 990.0 | | |
| White Opaque NP | 1 unit | 1500 units | Dosage | |
| Capsules Size 3 | | | Unit | |
| (Capsugel) | | | | |

**Note:
Particle size was obtained using a Malvern Mastersizer S. D (v, 0.1) = 0.52 μm/D (v, 0.5) = 9.04 μm/D (v, 0.9) = 22.55 μm.
^{φ}Note:
Particle size distribution was determined using a screening method taken form “Handbook of Pharmaceutical Excipients” 4^{th} edition by Raymond C. Rowe, Paul J. Weller, Paul J. Sheskey. APhA Publications (2003). ISBN: 1582120226.

### Procedure:

(1) 0.55 g Compound S-I (micronized) was combined with 1 g Starch 1500 and mixed with a spatula in a weighboat, then passed through a 40-mesh screen. Materials were passed through the sieve without any detectable residue remaining on the screen.

(2) Step 1 material was combined with 2 g of Starch 1500 and mixed with a spatula in a weighboat, then passed through a 40-mesh screen. Again, materials were passed through the sieve without any detectable residue remaining on the screen.

(3) An additional 5 g of Starch 1500 was mixed with the step 2 material in a weigh boat and mixed with a spatula then passed through a 40-mesh screen. Materials were passed through the sieve without any detectable residue remaining on the screen.

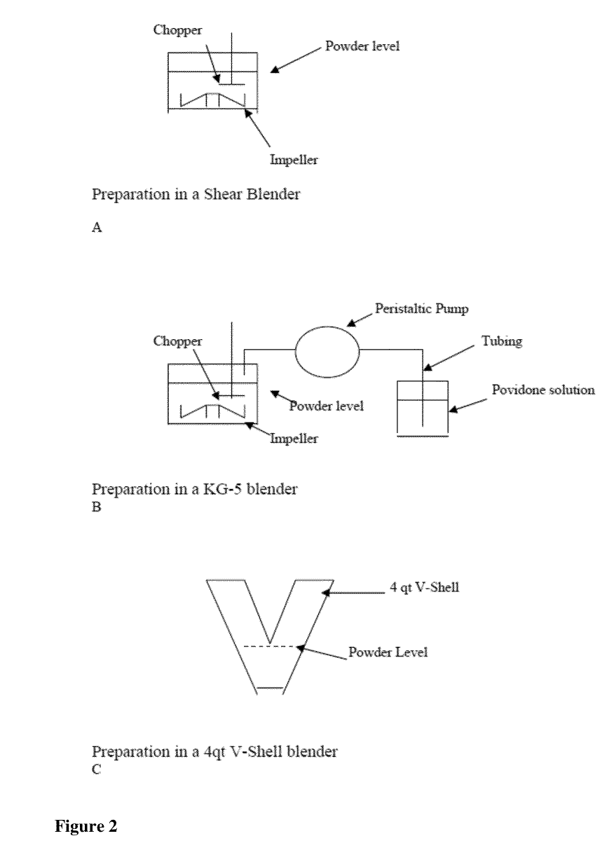

(4) Step 3 material along with all remaining excipients (except magnesium stearate) were sieved through a 20-mesh screen and placed into a KG-5 high shear blender as presented in FIG. 2B.

(5) Powders were blended using an impeller speed of 425 rpm and chopper speed at 1000 rpm for 10 minutes.

(6) Blend samples were taken in duplicate from top, middle, and bottom of the blender.

(7) Using the same settings as step 5 an additional 5 minutes of blending was performed and blend samples were taken in duplicate from top, middle, and bottom of the blender. This was repeated 4 more times (steps 8-11).

Magnesium stearate was screened through a 20-mesh screen and added to the blender. Due to increased powder volume in the KG-5, settings were adjusted to 325 rpm impeller speed and 950 rpm chopper speed and 5 minutes of blending was performed. Blend samples were taken in duplicate from top, middle, and bottom of the blender. A quantity of 1500 Size 3 capsules were filled with ˜180 mg of blend. Max. fill weight for batch=189 mg. Min. fill weight for batch =170 mg.

### B. High Shear Wet Granulation/Excipient Dilution Process

TABLE 2
Quantitative Pharmaceutical Composition for High
Shear Wet Granulation/Excipient Dilution Process

| | Target Weight/ | Weight/ | |
|---|---|---|---|
| INGREDIENTS | 180 (mg) | Batch (g) | Function |
| Compound S-I | 1.000 | 5.000 | Active |
| (Micronized) | | | |
| Pregelatinized | 8.000 | 40.00 | Diluent |
| Starch, NF | | | |
| (Starch 1500) | | | |
| Lactose Monohydrate, NF | 45.00 | 225.0 | Diluent |
| (Foremost 316 Fast-Flo) | | | |
| Microcrystalline | 41.20 | 206.0 | Diluent |
| Cellulose, NF | | | |
| (Avicel ® PH 102) | | | |
| Sodium Lauryl | 1.800 | 9.000 | Surfactant |
| Sulfate, NF | | | |
| PVP K-30, NF | 3.000 | 15.00 | Binder |
| TOTAL | 100.0 | 500.0 | |

Final Blend Additives (Dilution)

| | | | |
|---|---|---|---|
| Granulation Above | 10.00 | 55.00 | Active |
| Microcrystalline | 78.20 | 430.1 | Diluent |
| Cellulose, NF | | | |
| (Avicel ® PH 102) | | | |
| Lactose Monohydrate, NF | 90.00 | 495.0 | Diluent |
| (Foremost 316 Fast-Flo) | | | |
| Colloidal Silicon | 0.900 | 4.950 | Glidant |
| Dioxide, NF | | | |
| (Cab-O-Sil ® M5P) | | | |
| Magnesium Stearate, NF | 0.900 | 4.950 | Lubricant |
| (Non-bovine Hyqual ®) | | | |
| TOTAL | 180.0 | 990.0 | |
| White Opaque NP | 1 unit | 1300 units | Dosage |
| Capsules Size 3 | | | Units |
| (Capsugel) | | | |

TABLE 3
Quantitative Pharmaceutical Composition

| | Target Weight/ | Weight/ | |
|---|---|---|---|
| INGREDIENTS | 170 (mg) | Batch (g) | Function |
| Compound S-I | 1.000 | 5.000 | Active |
| (Micronized) | | | |
| Pregelatinized | 8.000 | 40.00 | Diluent |
| Starch, NF | | | |
| (Starch 1500) | | | |
| Lactose Monohydrate, NF | 45.00 | 225.0 | Diluent |
| (Foremost 316 Fast-Flo) | | | |
| Microcrystalline | 41.20 | 206.0 | Diluent |
| Cellulose, NF | | | |
| (Avicel ® PH 102) | | | |
| Sodium Lauryl | 1.800 | 9.000 | Surfactant |
| Sulfate, NF | | | |
| PVP K-30, NF | 3.000 | 15.00 | Binder |
| TOTAL | 100.0 | 500.0 | |

Final Blend Additives (Dilution)

| | | | |
|---|---|---|---|
| Granulation Above | 10.00 | 55.00 | Active |
| Microcrystalline | 78.20 | 430.1 | Diluent |
| Cellulose, NF | | | |
| (Avicel ® PH 102) | | | |
| Lactose Monohydrate, NF | 80.00 | 440.0 | Diluent |
| (Foremost 316 Fast-Flo) | | | |
| Colloidal Silicon | 0.900 | 4.950 | Glidant |
| Dioxide, NF | | | |
| (Cab-O-Sil ® M5P) | | | |
| Magnesium Stearate, NF | 0.900 | 4.950 | Lubricant |
| (Non-bovine Hyqual ®) | | | |
| TOTAL | 170.0 | 935.0 | |
| White Opaque Hard | 1 unit | 2000 units | Dosage |
| Gelatin Capsules | | | Units |
| Size 3 (Capsugel) | | | |

### Procedure:

5.0 g Compound S-I (micronized) was combined with 5 g Starch 1500 mixed with a spatula in a weighboat and passed through a 40-mesh screen. Materials were passed through the sieve without any detectable residue remaining on the screen.

The material was combined with 10 g of Starch 1500 and mixed with a spatula in a weighboat then passed through a 40-mesh screen. Again, the materials were passed through the sieve without any detectable residue remaining on the screen.

An additional 20 g of Starch 1500 was mixed with the material in a weigh boat and mixed with a spatula then passed through a 40-mesh screen. The mixture was passed through the sieve without any detectable residue remaining on the screen.

The material along with all remaining excipients (except magnesium stearate) was sieved through a 20-mesh screen and placed into a KG-5 high shear blender (as depicted in FIG. 2B). Powders were blended for 2 minutes using a chopper speed of 420 rpm (formulation according to Table 2) or 400 rpm (formulation according to Table 3) and impeller speed of 1000 rpm (formulation according to Table 2) or 800 rpm (formulation according to Table 3).

Polyvinylpyrrolidone (PVP K30) was dissolved in 285 g of purified water (USP water) forming a 5% solution. Using an impeller speed of 500 rpm (in formulation according to Table 2) or 400 rpm (in formulation according to Table 3), a chopper speed of 1000 rpm and a spray rate of 10 mL/min, PVP was added until a sufficient granule was obtained. Solution was added for 9.5 min (formulation according to Table 2) or 11.25 min (formulation according to Table 3). Total amount of solution added was 125 mL (in formulation according to Table 2) or 225 mL (in formulation according to Table 3).

Granules were dried in a fluid bed dryer until a limit of detection (LOD) <3.0% was reached. Final LOD end point 1.96%; total time 40 minutes (formulation according to Table 2) or final LOD end point 1.95%; total time 25 minutes (formulation according to Table 3). Granules were hand screened through a 20-mesh screen.

A portion of the granules (see Table 2 or 3) was placed in a 4 qt V-shell blender, as depicted in FIG. 2C. The final blend dilution was weighed out (see Table 2 or 3), screened through a 20-mesh screen (except magnesium stearate) and placed in the blender.

Materials were blended for 15 minutes. Blend samples were taken in duplicate from top, middle, and bottom of the blender.

Magnesium stearate was screened through a 20-mesh screen and added to blender. Materials were blended for an additional 5 minutes. Blend samples were taken in duplicate from top, middle, and bottom of the blender.

Table 2 shows 1300 size 3, NP capsules were filled with ˜180 mg of blend in each. Min. fill weight=165 mg; Max. fill weight=187 mg

Table 3 shows 2000 size 3, hard gelatin capsules were filled with ˜170 mg of blend in each. Min. fill weight=159 mg; Max. fill weight=178 mg

Samples were stored using the following packaging components:
Bottles: 120 cc Amber, Mfr. Owens-Brockway
Rayon Pharmaceutical Coil: Mfr. Carolina Absorbent Cotton
Cap: 38 mm white, IS, Mfr. Mold-Rite Plastics

C. Direct Blend and Fill into Size 3 Capsules

TABLE 4
Quantitative Pharmaceutical Composition for
Direct Blend and Fill into Size 3 Capsules

| | Target Weight/ | Weight/ | |
|---|---|---|---|
| INGREDIENTS | 180 (mg) | Batch (g) | Function |
| Compound S-I | 0.100 | 0.5500 | Active |
| (Micronized) | | | |
| Pregelatinized | 8.000 | 44.00 | Diluent |
| Starch, NF | | | |
| (Starch 1500) | | | |
| Lactose Monohydrate, NF | 93.30 | 513.1 | Diluent |
| (Foremost 316 Fast-Flo) | | | |
| Microcrystalline | 75.00 | 412.5 | Diluent |
| Cellulose, NF | | | |
| (Avicel ® PH 102) | | | |
| Sodium Lauryl | 1.800 | 9.900 | Surfactant |
| Sulfate, NF | | | |
| Colloidal Silicon | 0.900 | 4.950 | Glidant |
| Dioxide, NF | | | |
| (Cab-O-Sil ® M5P) | | | |
| Magnesium Stearate, NF | 0.900 | 4.950 | Lubricant |
| (Non-bovine Hyqual ®) | | | |
| TOTAL | 180.0 | 990.0 | |
| White Opaque NP | 1 unit | 1000 units | Dosage Unit |
| Capsules | | | |
| Size 3 (Capsugel) | | | |

TABLE 5
Quantitative Pharmaceutical Composition for
Direct Blend and Fill into Size 3 Capsules

| | Target Weight/ | Weight/ | |
|---|---|---|---|
| INGREDIENTS | 180 (mg) | Batch (g) | Function |
| Compound S-I | 0.300 | 0.5500 | Active |
| (Micronized) | | | |
| Pregelatinized | 8.000 | 44.00 | Diluent |
| Starch, NF | | | |
| (Starch 1500) | | | |
| Lactose Monohydrate, NF | 93.10 | 513.1 | Diluent |
| (Foremost 316 Fast-Flo) | | | |
| Microcrystalline | 75.00 | 412.5 | Diluent |
| Cellulose, NF | | | |
| (Avicel ® PH 102) | | | |
| Sodium Lauryl | 1.800 | 9.900 | Surfactant |
| Sulfate, NF | | | |
| Colloidal Silicon | 0.900 | 4.950 | Glidant |
| Dioxide, NF | | | |
| (Cab-O-Sil ® M5P) | | | |
| Magnesium Stearate, NF | 0.900 | 4.950 | Lubricant |
| (Non-bovine Hyqual ®) | | | |
| TOTAL | 180.0 | 990.0 | |
| White Opaque NP | 1 unit | 1000 units | Dosage Unit |
| Capsules | | | |
| Size 3 (Capsugel) | | | |

TABLE 6
Quantitative Pharmaceutical Composition

| | Target Weight/ | Weight/ | |
|---|---|---|---|
| INGREDIENTS | 180 (mg) | Batch (g) | Function |
| Compound S-I | 0.100 | 0.5500 | Active |
| (Micronized) | | | |
| Pregelatinized | 8.000 | 44.00 | Diluent |
| Starch, MF | | | |
| (Starch 1500) | | | |
| Lactose Monohydrate, NF | 83.30 | 458.2 | Diluent |
| (Foremost 316 Fast-Flo) | | | |
| Microcrystalline | 75.00 | 412.5 | Diluent |
| Cellulose, NF | | | |
| (Avicel ® PH 102) | | | |
| Sodium Lauryl | 1.800 | 9.900 | Surfactant |
| Sulfate, MF | | | |
| Colloidal Silicon | 0.900 | 4.950 | Glidant |
| Dioxide, NF | | | |
| (Cab-O-Sil ® M5P) | | | |
| Magnesium Stearate, NF | 0.900 | 4.950 | Lubricant |
| (Non-bovine Hyqual ®) | | | |
| TOTAL | 170.0 | 935.1 | |
| White Opaque Hard | 1 unit | 2000 units | Dosage Unit |
| Gelatin Capsules | | | |
| Size 3 (Capsugel) | | | |

Procedure:

0.5500 g of Compound S-I (micronized) was combined with 1 g Starch 1500 and mixed with a spatula in a weighboat, then passed through a 40-mesh screen. Materials were passed through the sieve without any detectable residue remaining on the screen.

Material was combined with 2 g of Starch 1500 and mixed with a spatula in a weighboat, then passed through a 40-mesh screen. Again, materials were passed through the sieve without any detectable residue remaining on the screen.

An additional 5 g of Starch 1500 was mixed with material in a weigh boat and mixed with a spatula then passed through a 40-mesh screen. Materials were passed through the sieve without any detectable residue remaining on the screen.

Material along with all remaining excipients (except magnesium stearate) was sieved through a 20-mesh screen and placed into a 4 qt V-shell blender (as depicted in FIG. 2C). Powders were blended for 10 minutes. Blend samples were taken in duplicate from top, middle, and bottom of the blender.

Powders were blended for an additional 5 minutes. Blend samples were taken in duplicate from top, middle, and bottom of the blender.

Powders were blended for an additional 5 minutes. Blend samples were taken in duplicate from top, middle, and bottom of the blender.

Powders were blended for an additional 5 minutes. Blend samples were taken in duplicate from top, middle, and bottom of the blender.

Magnesium Stearate was screened through a 20-mesh screen and added to blender. Materials were blended for an additional 5 minutes. Blend samples were taken in duplicate from top, middle, and bottom of the blender.

1000 size 3, NP capsules were filled with ˜180 mg of blend in each (according to the formulation of Tables 4 and 5). Min. fill weight=168 mg; Max. fill weight=189 mg.

2000 size 3, hard gelatin capsules were filled with ˜170 mg of blend in each (According to the formulation of Table 6). Min. fill weight=160 mg; Max. fill weight=177 mg.

Samples were stored using the following packaging components:
Bottles: 120 cc Amber, Mfr. Owens-Brockway
Rayon Pharmaceutical Coil: Mfr. Carolina Absorbent Cotton
Cap: 38 mm white, IS, Mfr. Mold-Rite Plastics.

### 10 mg S-I Capsules

The process of preparation of 10 mg S-I Capsules is as described above using V-shell blender. The 10 mg S-I Capsules is about 340 mg weight (about 260 mg fill weight and about 80 mg empty capsule weight).

The dissolution results of 10 mg S-I Capsules are provided herein below:

| time | Dissolution l.c. %-mean (% RSD) |
|---|---|
| 15 min | 86%, (4.9) |
| 30 min | 90%, (2.5) |
| 45 min | 92%, (1.9) |
| 60 min | 92%, (2.1) |

The moisture report indicated about 4.8% w/w

The certificate of Analysis of 10 mg S-I Capsules, indicated the following:

| Test | Specification |
|---|---|
| Physical Description | Size No. 1, opaque, hard |
| | gelatin capsule containing |
| | off-white to light tan powder |
| | with no visible |
| | contamination |
| Identification by HPLC | The retension time for S-I in |
| | the sample corresponds to |
| | the retention time for S-I in |
| | the reference standard |
| | within ± 0.5 minutes |
| Impurity (HPLC) Content | 99.5% l.c. S-I |
| | The following specific |
| | impurities were not detected: |
| | CK-153; |
| | 4-hydroxybenzonitrile and |
| | 4-cyano-3-(trifluoromethyl)aniline |

Compound S-I was sifted through 20-mesh screen and micronized using Jet-O-Mill micronizer. Half of the Starch 1500 was sifted through a 20-mesh screen and placed in the blender.

Samples were submitted for analysis to obtain content uniformity and for drug product method development purposes. The above described capsules represent embodiments of pharmaceutical composition of this invention.

### EXAMPLE 4

### Pharmaceutical Compositions

### Capsules Comprising Compound S-I, 1 mg and 3 mg

The active ingredient was Compound S-I. The inactive ingredients were pregelatinized starch, lactose monohydrate, microcrystalline cellulose, sodium lauryl sulfate, colloidal silicon dioxide and magnesium stearate The blended active and inactive ingredients are filled into Number-one (#1) capsules. Capsules of Compound S-I were manufactured in accordance with the flow chart depicted in FIG. 1, using the formulations as set forth in Table 7.

TABLE 7
Quantitative Pharmaceutical Composition

| | Composition of Batch (grams) | | | | | |
|---|---|---|---|---|---|---|
| | Placebo | | 1 mg | | 3 mg | |
| | Capsule | Batch | Capsule | Batch | Capsule | Batch |
| Component | (mg) | (g) | (mg) | (g) | (mg) | (g) |
| Compound S-I, | 0 mg | 0 mg | 1.0 | 64.00 | 3.0 | 192.00 |
| micronized | | | | | | |
| Pregelatinized Starch | 80.0 | 5,120 | 80.0 | 5,120 | 80.0 | 5,120 |
| Lactose Monohydrate | 112.5 | 7,200 | 111.5 | 7,136 | 109.5 | 7,008 |
| Microcrystalline | 40.0 | 2,560 | 40.0 | 2,560 | 40.0 | 2,560 |
| Cellulose | | | | | | |
| (Avicel ® PH 102) | | | | | | |
| Sodium Lauryl | 30.0 | 1920 | 30.0 | 1920 | 30.0 | 1920 |
| Sulfate | | | | | | |
| Colloidal Silicon | 1.0 | 64.00 | 1.0 | 64.00 | 1.0 | 64.00 |
| Dioxide | | | | | | |
| (Cab-O-Sil ® MSP) | | | | | | |
| Magnesium Stearate | 1.5 | 96.00 | 1.5 | 96.00 | 1.5 | 96.00 |
| Total | 265.0 | 16,960 | 265.0 | 16,960 | 265.0 | 16,960 |
| Number of Capsules | | 64,000 | | 64,000 | | 64,000 |

Compound S-I was sieved through a 20-mesh screen. A Jet-O-Mill Micronizer is used to produce a nominal particle size of 5 μm. All components, including micronized Compound S-I, were weighed into individual containers. Approximately one-half of the Pregelatinized Starch, NF were sifted through a 20-mesh screen; placed into the 16 qt. v-blender; and mixed for one minute to coat the internal surfaces of the blender. The micronized Compound S-I drug substance is sifted through a 40-mesh sieve and added to the blender. The remaining Pregelatinized Starch, NF, was sifted through a 40-mesh sieve and added to the blender. Lactose monohydrate 316, sodium lauryl sulfate, microcrystalline cellulose colloidal, and silicone dioxide are sifted through a 20-mesh sieve and added to the blender. The combined contents were mixed for approximately 15 minutes. Finally, magnesium stearate was sifted through a 20-mesh sieve and added to the blender (with a portion of the premixed blend, if necessary). The combined contents were mixed for approximately 5 minutes. Once formulated, the mixed components were fed into the automatic capsule filler, Bohannon Capsule Filler. Number-one (#1) capsules were filled to the same target weight (265.0 mg) for the 1 mg and 3 mg actives as well as the placebo.

Weight checks were conducted on randomly selected capsules that are collected in 10-minute intervals. The filled capsules are dedusted using a capsule polisher and visually inspected. Samples from the beginning, approximate middle and end of the filing/capping process are taken for analysis. The remaining capsules are placed into containers lined with two polyethylene bags and stored at 15-25° C.

### EXAMPLE 5

### Pharmaceutical Compositions

### Tablets Comprising Compound S-I, 0.1 mg, 0.3 mg, 1 mg and 3 mg

Tablets of Compound S-I were manufactured using the formulations as set forth in Table 8.

TABLE 8
Quantitative Pharmaceutical Composition

| | 0.1 mg dose | 0.3 mg dose | 1 mg dose | 3 mg dose |
|---|---|---|---|---|
| | (mg/tablet) | (mg/tablet) | (mg/tablet) | (mg/tablet) |
| Material | | | | |
| Compound S-I | 0.100 | 0.300 | 1.000 | 3.000 |
| Pregelatinized | 5.000 | 5.000 | 5.000 | 5.000 |
| Starch, NF | | | | |
| (Starch 1500) | | | | |
| Lactose monohydrate, | 57.90 | 57.70 | 57.00 | 55.00 |
| NF(Foremost 316) | | | | |
| Microcrystalline | 35.00 | 35.00 | 35.00 | 35.00 |
| Cellulose, NF | | | | |
| (Avicel ® PH 102) | | | | |
| Sodium Lauryl | 1.000 | 1.000 | 1.000 | 1.000 |
| Sulfate, NF | | | | |
| (Spectrum) | | | | |
| Colloidal silicon | 0.500 | 0.500 | 0.500 | 0.500 |
| dioxide, NF | | | | |
| (Cab-O-Sil ® M5P | | | | |
| Magnesium Stearate, NF | 0.500 | 0.500 | 0.500 | 0.500 |
| (Non-bovine Hyqual ®) | | | | |
| Ethyl alcohol, USP | 0.000 | 0.000 | 0.000 | 0.000 |
| (evaporated in process) | | | | |
| Total | 100.0 | 100.0 | 100.0 | 100.0 |
| Physical Attributes | | | | |
| Mean hardness | 6.2 | 4.5 | 4.6 | 4.8 |
| (n = 10) (kp) | | | | |
| % Loss Friability | 0.03% | 0.03% | 0.08% | 0.03% |
| Capping in friabilator | No | No | No | No |
| (Y/N) | | | | |
| Disintegration | 1:29 | 0:30 | 1:04 | 1:28 |
| (n = 6)(min:sec) | | | | |
| Mean Weight | 104.3 | 104.0 | 103.6 | 103.5 |
| (n = 10) (mg) | | | | |
| Individual Weight % | 1.24% | 0.94% | 0.85% | 0.70% |
| RSD | | | | |
| Thickness (mm) | 3.91 | 3.97 | 3.93 | 3.99 |

### Procedure:

The ingredients listed in Table 8 were weighed (except the alcohol).

All powders were sifted except the active CabOSil and the magnesium stearate through a 20-mesh screen into the KG-5 granulator. The powders were blended dry with the impeller at 250 rpm for 2 minutes. The active pharmaceutical ingredient (API, Compound S-I) was dissolved in the ethanol, and was granulated by adding the granulation solvent to the powder bed.

The granules were dried in an oven or fluid bed dryer until limit of detection (LOD) <2.0%.

The granules were mill dried using the Quadro Comil with a 0.045″ round hole screen.

The milled granule was placed in a 2-quart V-shell type blender. Magnesium stearate and CabOSil were screened through a 20-mesh screen, added to the blender, and blended for 5 minutes.

The final blend was compressed on a force fed rotary tablet and pressed at a target weight of 100 mg using 5.5 mm round.

### EXAMPLE 6

### Pharmacology of Compound S-I and Alendronate

### SARM Bone Effects Alone and in Combination with the Anti-Resorptive Agent, Alendronate

### Materials and Methods:

Sixty female, virgin, intact Sprague-Dawley rats were obtained from Charles River Laboratories (Wilmington, Mass.) and aged to 23 weeks. The animals were housed 2-3 per cage and acclimated to a 12 h light/dark cycle. Food (7012C LM-485 Mouse/Rat Sterilizable Diet, Harlan Teklad, Madison, Wis.) and water were provided ad libitum.

Sham surgeries or ovariectomies were performed on Day 0. The study was comprised of eight treatment groups as follows: (1) intact +vehicle, (2) intact +Compound S-I, (3) OVX+vehicle (4) OVX+Compound S-I, (5) OVX+dihydrotestosterone (DHT), (6) OVX+estradiol (E2), (7) OVX+alendronate, (8) OVX+alendronate+Compound S-I. Doses were administered daily via oral gavage in a vehicle of DMSO:PEG300 (10:90) beginning on Day 1. Animals were sacrificed on Day 45 of the study. Femurs were removed, cleared of soft tissue, and stored in saline soaked gauze at −20° C. until analysis. Nine animals died during the course of the study. These deaths were attributed to surgical complications arising from the ovariectomies and technical errors during oral dosing (i.e., dosing solution delivered into the lungs). Dose groups are listed in Table 9 below:

TABLE 9

| | Gonadal | | | | Animals/ |
|---|---|---|---|---|---|
| Group | Status | Treatment | Dose | | group |
| 1 | Intact | Vehicle | N/A | | 9 |
| 2 | Intact | Compound S-I | 3 | mg/day | 9 |
| 3 | OVX | Vehicle | N/A | | 7 |
| 4 | OVX | Compound S-I | 3 | mg/day | 8 |
| 5 | OVX | Alendronate | 1 | mg/day | 10 |
| 6 | OVX | Alendronate/ | 1 and 3 | mg/day | 8 |
| | | Compound S-I | | | |

The left femurs were sent to SkeleTech Inc. (Bothell, Wash.) for biomechanical strength (three point bending) and pQCT analysis. A Stratec XCT RM and associated software (Stratec Medizintechnik GmbH, Pforzheim, Germany, software version 5.40 C) were used for the pQCT analysis. The femur was analyzed at both the mid-shaft and distal regions. The mid-shaft analysis was performed on the region at 50% of the length of the femur. The distal analysis was performed on the region at 20% of the length of the femur starting at the distal end. One 0.5 mm slice perpendicular to the long axis of the femur was used for analysis. Total bone mineral content, total bone area, total bone mineral density, cortical bone mineral content, cortical bone area, cortical bone mineral density, cortical thickness, periosteal perimeter (circumference) and endosteal perimeter were determined at the mid-shaft of the femur. At the distal femur, total bone mineral content, total bone area, total bone mineral density, trabecular bone mineral content, trabecular bone area and trabecular bone mineral density were determined Following pQCT analysis, the femoral strength was determined by a three-point bending test. The anterior to posterior diameter (APD) (unit:mm) at the midpoint of the femoral shaft was measured with an electronic caliper. The femur was placed on the lower supports of a three-point bending fixture with the anterior side of the femur facing downward in an Instron Mechanical Testing Machine (Instron 4465 retrofitted to 5500) (Canton, Mass.). The length (L) between the lower supports was set to 14 mm The upper loading device was aligned to the center of the femoral shaft. The load was applied at a constant displacement rate of 6 mm/min until the femur broke. The mechanical testing machine directly measured the maximum load (Fᵤ) (unit:N), stiffness (S) (units:N/mm), and energy absorbed (W) (unit:mJ). The axial area moment of inertia (I) (unit:mm⁴) was calculated by the software during the pQCT analysis of the femoral mid-shaft. Stress (σ) (units:N/mm²), elastic modulus (E) (unit:Mpa), and toughness (T) (units:mJ/m³) were calculated by the following formulas: stress: σ=(Fᵤ*L*(a/2))/(4*I); elastic modulus: E=S*L³/(48*I); and toughness: T=3*W*(APD/2)²/(L*I).

Statistical analysis was performed by Student's T-test. P-values of less than 0.05 were considered as statistically significant differences.

Male rats were subjected to orchiectomy (ORX), and on days 1-119 were administered perorally by gavage a vehicle, different doses of Compound S-I (0.1, 1, and 3 mg/d), with or without alendronate (1 mg/d), and alendronate alone. After sacrifice at the indicated times, mice were sacrificed, femurs removed and subjected to pQCT analysis and a 3-point bending assay. Vertebra were harvested as well, and crush assay of L5 was conducted. Tibias were subjected to static and dynamic histomorphometry (calcein).

### Results:

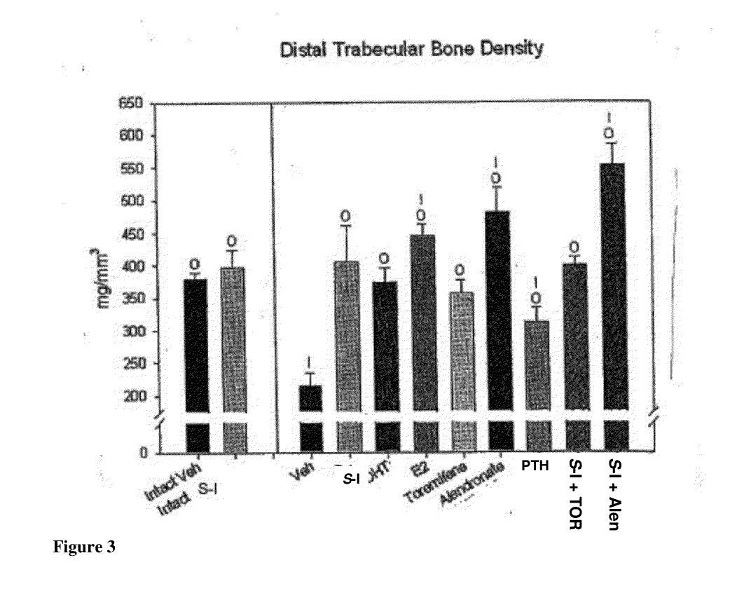

Trabecular bone mineral density was analyzed by pQCT at the distal femur. Results are shown in FIG. 3. Significant trabecular bone loss was observed following OVX. Trabecular bone density decreased from 379 to 215 mg/mm³ in the intact and OVX vehicle control groups, respectively. In intact animals treated with Compound S-I, a slight increase in trabecular bone density to 398 mg/mm³ was observed. In OVX animals treated with Compound S-I, a significant increase was observed over the OVX vehicle control group to 406 mg/mm³ DHT increased trabecular bone density over the OVX vehicle control group to 360 mg/mm³ and estradiol (E2) increased trabecular bone density to 415 mg/mm³ Alendronate increased trabecular bone density to 480 mg/mm³ The combination therapy of alendronate and Compound S-I showed additive effects increasing trabecular bone density to 552 mg/mm³

### EXAMPLE 7

### Pharmaceutical Compositions

### Softgel Capsules Comprising Compound S-I, 0.5 mg, 1 mg, or 3 mg

S-I was developed as a formulated soft gelatin capsule (softgel) with a dosage strength of 3.0 mg for Phase III clinical studies. S-I 3.0 mg softgel drug product was provided as an opaque, white to off-white, size 5, oval softgel with “GTx” imprinted in black on the outer shell. The product was packaged in a 35-count white high-density polyethylene (HDPE) 60 cc bottle with a child-resistant closure and an induction seal.

### Composition of the Drug Product

All components used in the manufacture of the product and their functions are provided in Table 10.

Component

S-I
Polyethylene Glycol 400
Gelatin (Type 195)
Sorbitol Special -
Glycerin Blend A810
Titanium Dioxide
Black Opacode WB
Nitrogen
Lecithin, Unbleached
Fractionated Coconut Oil
Denatured Ethanol with
Isopropyl Alcohol
Phosal ®53 MCT

The composition of S-I 1 mg, 3 mg and placebo (powder-filled) capsules is given in Table 11 (Example 4).

TABLE 11
Composition of S-I, 1 mg, 3 mg, and Placebo Capsules
Component

S-I, micronized
Pregelatinized Starch
Lactose Monohydrate
Microcrystalline
Cellulose (Avicel ® PH 102)
Sodium Lauryl Sulfate
Colloidal Silicon
Dioxide (Cab-O-Sil ® MSP)
Magnesium Stearate

### Quantitative Composition

The quantitative composition of S-I 3.0 mg softgels is described in Table 12.

TABLE 12

| | |
|---|---|
| | Unit Formula |
| Component | (mg/softgel) |

A: Batch Formula S-I 1.0 mg softgels

| | |
|---|---|
| S-I | 1.0 |
| Polyethylene Glycol 400, NF, EP | 299.0 |
| Total Weight | 300.0 |

B: Batch Formula S-I 3.0 mg softgels

| | |
|---|---|
| S-I | 3.0 |
| Polyethylene Glycol 400, NF, EP | 297.0 |
| Total Weight | 300.0 |

C: Batch Formula S-I 0.5 mg softgels

| | |
|---|---|
| S-I | 0.5 |
| Polyethylene Glycol 400, NF, EP | 299.5 |
| Total Weight | 300.0 |

The estimated mass range of soft gelatin shells is presented in Table 13. The actual weight of shell mass may vary based on environmental conditions and the hydrophilic nature of the fill material.

TABLE 13
Composition of Soft Gelatin Shells*

| | Unit Formula |
|---|---|
| Component | (g/softgel) |
| Gelatin (Type 195) NF, EP | 0.0841-0.0988 |
| Sorbitol Special - | 0.0531-0.0624 |
| Glycerin Blend A810 | |
| Titanium Dioxide | 0.0011-0.0013 |

*Estimated dried shell mass

### EXAMPLE 8

### Process of Manufacturing Softgels

### Description of Manufacturing Process and Process Controls for S-I, 3.0 mg, softgels

### Manufacturing Process

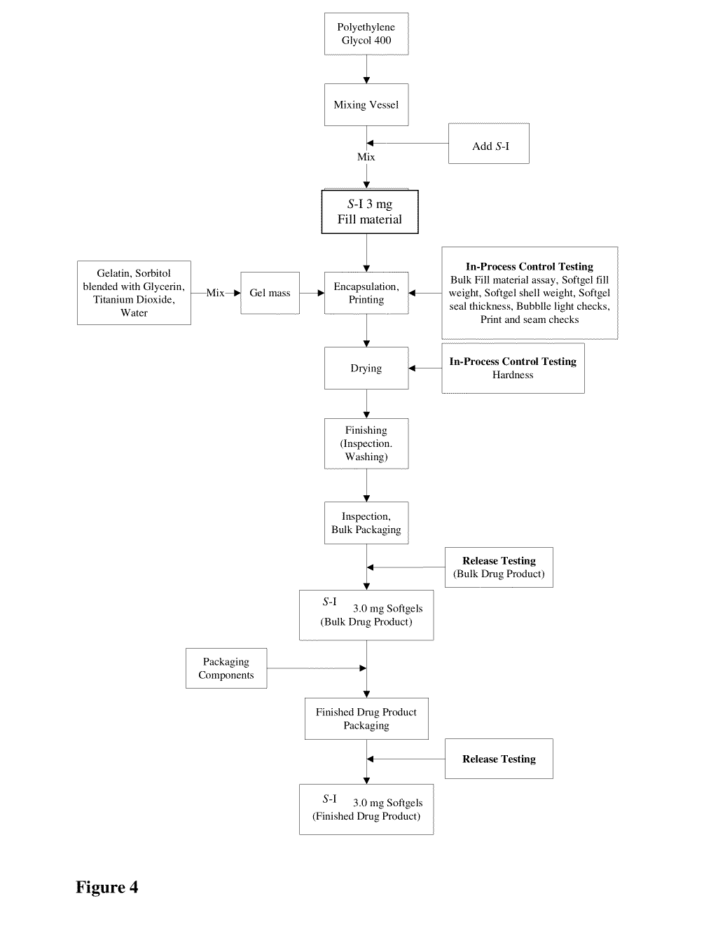

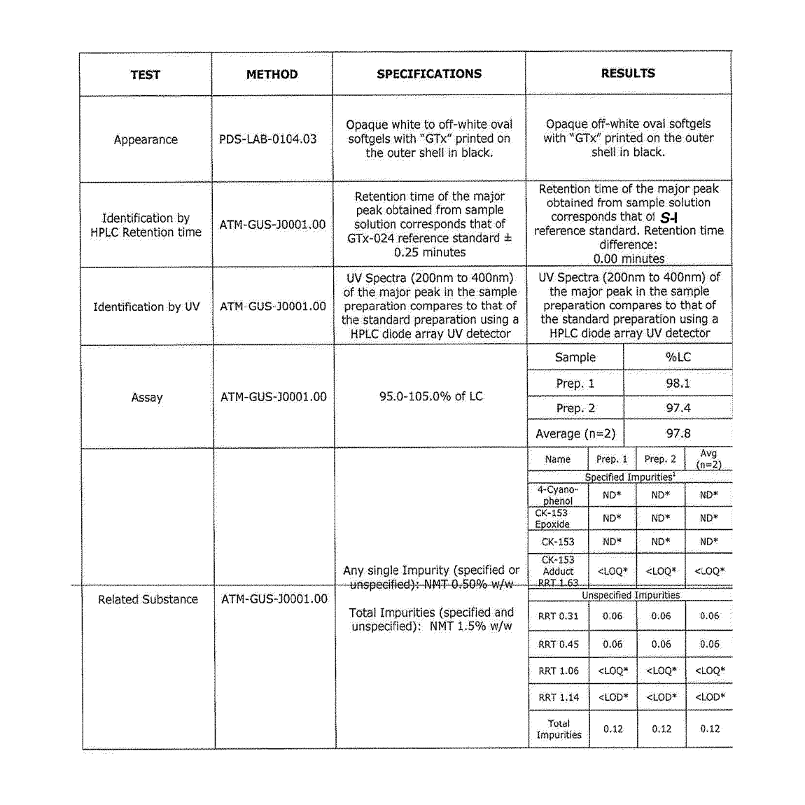

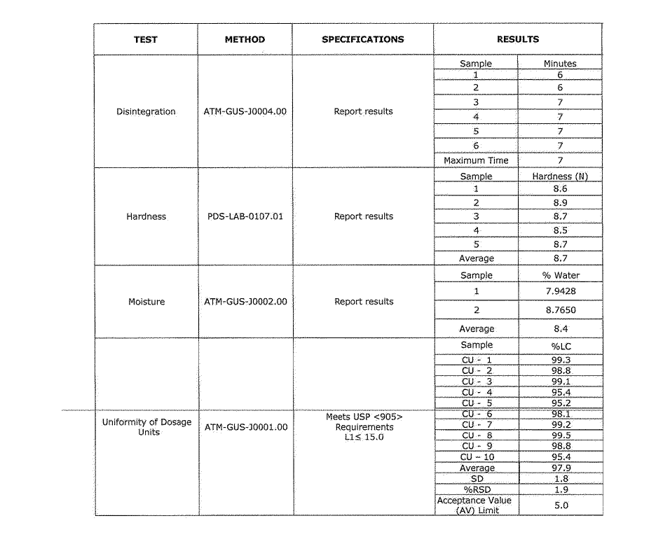

A flow diagram for the manufacturing process for the S-I softgels is provided in FIG. 4.

The manufacturing process of S-I softgels consists of the preparation of gel mass, preparation of S-I fill material, encapsulation and printing, drying, finishing, inspection and bulk packaging.

### 1. Preparation of Gel Mass

Gelatin, sorbitol (blended with glycerin), water and titanium dioxide were mixed together according to Catalent's proprietary formulation and manufacturing process for gel preparation.

### 2. Preparation of S-I Fill Material

Polyethylene Glycol 400 was weighed and placed into an appropriate mixing vessel. S-I was weighed and added to the mixing vessel. The mixture was mixed under vacuum for not less than 30 minutes until S-I was completely dissolved. The mixing was then stopped and the mixture was allowed to stand for deaeration. The mixing vessel was then vented with nitrogen. The mixture was stored under nitrogen blanket prior to encapsulation.

### 3. Encapsulation and Printing

Encapsulation of the S-I fill material was performed on a rotary die encapsulation machine with the target in-process fill weight of 0.306 g. Due to the hygroscopic nature of the fill material, the in-process fill weight was increased by 2% from the theoretical fill weight of 0.300 g. The outer shell of the softgel was then imprinted with “GTx.”

### 4. Drying

The softgels were tray-dried in drying tunnels to the hardness specification of 9.0-11.0 Newton (N).

### 5. Finishing (Inspection and Washing)

Once the required hardness was achieved, the softgels were visually inspected and washed on a spray washer with denatured ethanol/Phosal wash solution.

### 6. Inspection and Bulk Packaging

The washed softgels were graded through a sizing device to remove any gross over and undersized softgels. The graded softgels were then transported across a conveyor for visual inspection, and finally passed through a metal detector. The bulk product was then sampled and packaged in standard cartons with a polyethylene carton liner. Samples were submitted for release testing. The bulk product was stored at 15-30° C. with not more than 50% RH.

### Analytical Procedures

Analytical Procedures (S-I softgels)

### Appearance.

S-I softgels were visually inspected for appearance: color, shape and physical description.

### Identification by HPLC, Assay, Related Substances.

Identification, assay and related substances (impurities) were determined by HPLC analysis using a YMC Pack Pro C18 RS, 4.6 x 150 mm, 5 μm particle size column with UV detection at 210 nm Samples of S-I softgels were prepared at 0.018 mg/mL analytical concentration and chromatographed by reversed phase gradient method using acetonitrile and 25 mM potassium phosphate, monobasic solution. Assay and related substances were quantified on a %w/w basis against an external reference standard. Identity was confirmed with the chromatographic retention time of the major peak obtained from a sample solution corresponding to that of S-I reference standard within ±0.25 minutes.

### Identification by UV.

Identification S-I by UV was conducted as part of the HPLC assay procedure. The UV spectra of the S-I softgels sample was compared with that of the standard to confirm its identity. Positive identification was confirmed when the UV spectra (210-400 nm) of the main peak in the sample solution corresponded to the spectra of S-I in the standard solution. Dissolution.

S-I softgels were analyzed for dissolution using a USP Apparatus II with 500 mL 0.1% Hexadecyl Trimethyl Ammonium Bromide (CTAB) in 0.01 N hydrochloric acid at 37.0° C.±0.5° C., at 50 rpm at time points 10, 20, 30, and 60 minutes. Five-mL aliquots were analyzed by HPLC using a YMC-Pack Pro C18 RS, 150 mm×4.6 mm, S-5 μm, 8 nm column with UV detector at 210 nm, using a reversed phase isocratic method, with acetonitrile and 25 mM potassium phosphate, monobasic solution as a mobile phase.

### Disintegration.

Disintegration of S-I softgels was determined in 0.01 N hydrochloric acid as per current USP disintegration <701>.

### Hardness.

Hardness of S-I softgels was determined using Baresis Durometer. The measurements are reported in Newton (N).

### Moisture.

S-I softgels were analyzed for moisture by Karl Fischer titration.

### Content Uniformity.

Content Uniformity of S-I softgels were analyzed by HPLC using a YMC Pack Pro C18 RS, 4.6×150 mm, 5 μm particle size column with UV detection at 210 nm with a reversed phase gradient method using acetonitrile and 25 mM potassium phosphate, monobasic solution.

### Microbiological Testing.

S-I softgels were tested for microbiological quality. Total aerobic microbial count and total combined yeasts/molds count were controlled in accordance with USP <61>, <62> requirements for Microbiological examination of non-sterile products. Identification of any growth obtained was done to a level that an objectionable organism could be ruled out, following Microbiology Laboratory General Guidelines and Practices as per USP <1111>.

### Analytical Procedures (S-I, Capsules, 1 mg, 3 mg, and Placebo)

Each of the methods used to control the drug substance are listed and summarized below.

### Physical Description.

S-I 1 mg, 3 mg and placebo (powder-filled) capsule shell and contents were visually inspected for appearance, color and physical condition.

### Assay and Impurities Content (HPLC).

Assay and impurities of S-I 1 mg, 3 mg and placebo capsules were quantitated using a Supelco Supelcosil ABZ+PLUS, 5 μm reverse phase column with UV detection at 215 nm Samples of S-I 1 mg and 3 mg capsules were prepared at 0.15 mg/mL and chromatographed using an acetonitrile (1 mg/L TFA) and water (1 mg/L TFA).

### Identification: HPLC.

The chromatographic retention times of the main peak for the S-I 1 mg and 3 mg capsules (for the above analysis) were compared to the reference standard. Identity was confirmed with a retention time of the reference standard peak within 0 5 minute of the main peak for the sample. Placebo capsules were similarly prepared and analyzed in the above analysis (15 placebo capsules in 100 mL diluent). Identity of the placebo capsule was confirmed by the absence of a peak corresponding to S-I.

### Content Uniformity.

Content uniformity of 1 mg and 3 mg S-I capsules was determined as per USP<905>. S-I was quantitated using the above assay and impurity chromatographic conditions with each sample preparation at 0.10 mg/mL.

### Dissolution.

S-I 1 mg and 3 mg capsule dissolution was determined using a USP Rotating Paddle (Apparatus II) at 75 rpm with 900 mL of 0.25% hexadecyltrimethylammonium bromide in pH 6.8 phosphate buffer. Ten ml aliquots were removed at 15, 30, 45 and 60 minutes and analyzed with the above Assay and Impurities HPLC method.

### Moisture.

S-I 1 mg, 3 mg and placebo capsules were analyzed for moisture by Karl Fischer titration.

### Batch Analysis

Batch Analysis (S-I 3.0 mg softgels)

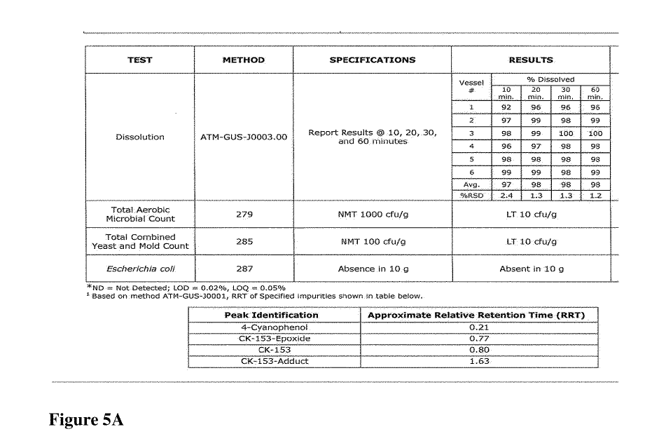

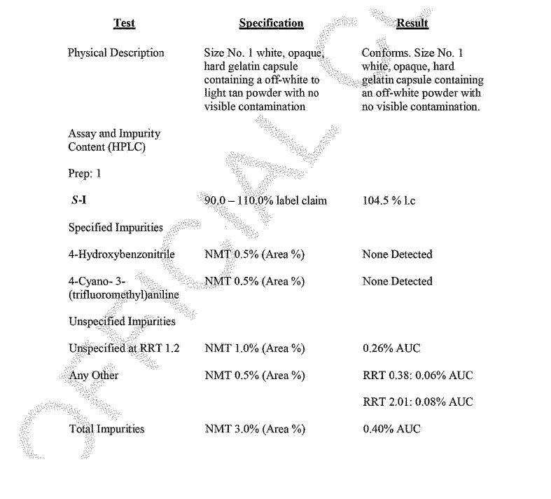

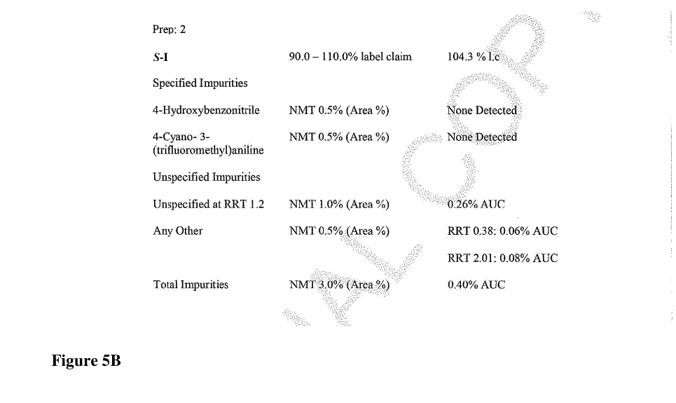

Batch analysis data for S-I 3.0 mg softgels are provided in Table 14. The batch was manufactured according to the details described in the section of Example 8 titled “Description of Manufacturing Process and Process Controls” presented above, and tested by the methods described in the section of Example 8 titled “Analytical Procedures” presented above. The Certificate of Analysis is shown in FIG. 5A.

TABLE 14
Batch Analysis Data for S-I 3.0 mg Softgels

| | | Development, | Clinical, |
|---|---|---|---|
| Usage | | Stability | Stability |
| Test | Specification | Batch Results | Batch Results |
| Identification | Retention time of the major | — | Conforms to |
| by HPLC | peak obtained from sample | | reference |
| Retention time | solution corresponds that of | | standard with a |
| | S-I reference standard ± 0.25 | | difference |
| | minutes | | of 0.0 minutes |
| Identification | UV Spectra (200 nm to 400 nm) | — | Conforms |
| by UV | of the major peak in the | | |
| | sample preparation compares | | |
| | to that of the standard | | |
| | preparation using a HPLC | | |
| | diode array UV detector | | |
| Assay (HPLC) | 95.0-105.0% label claim | 101.8% | 98.1% |
| | | 102.6% | 97.4% |
| | | Average = 102.2% | Average = 97.8% |
| Total Impurities | Total Impurities(specified | 0.20 | 0.12, 0.12 |
| (% w/w) | and unspecified): NMT 1.5% w/w | | |
| Dissolution | | | |
| 10 minutes | Report Results | 93, 98, 96, 96, 97, 95 | 92, 97, 98, 96, 98, 99 |
| (% Dissolved) | | Average = 96 | Average = 97 |
| | | % RSD = 1.9 | % RSD = 2.4 |
| 20 minutes | Report Results | Not performed at 20 | 96, 99, 99, 97, 98, 99 |
| (% Dissolved) | | minutes | Average = 98 |
| | | At 15 minute (% | % RSD = 1.3 |
| | | dissolved): 97, 98, | |
| | | 97, 97, 97, 96 | |
| | | Average = 97 | |
| | | % RSD = 0.5 | |
| 30 minutes | Report Results | 97, 98, 97, 97, 97, 97 | 96, 98, 100, 98, 98, 98 |
| (% Dissolved) | | Average = 97 | Average = 98 |
| | | % RSD = 0.5 | % RSD = 1.3 |
| 60 minutes | Report Results | 98, 98, 98, 98, 98, 98 | 96, 99, 100, 98, 98, 99 |
| (% Dissolved) | | Average = 98 | Average = 98 |
| | | % RSD = 0.3 | % RSD = 1.2 |
| Disintegration | Report Results | — | 6, 6, 7, 7, 7, 7 |
| (minutes) | | | Maximum time 7 minutes |
| Hardness (N) | Report Results | 9.1, 9.1, 9.2, 8.9, 8.8 | 8.6, 8.9, 8.7, 8.5, 8.7 |
| | | Average = 9.0 | Average = 8.7 |
| Moisture (% w/w) | Report Results | 7.5%, 7.5% | 7.9%, 8.8% |
| | | Average = 7.5% | Average = 8.4% |
| Content Uniformity | Meets USP <905> | — | 99.3, 98.8, 99.1, 95.4, |
| | Requirements | (Assay of fill | 95.2, 98.1, 99.2, 99.5, |
| | | material prior to | 98.8, 95.4% l.c. |
| | | encapsulation: | Average = 97.9% l.c. |
| | | 100.1% l.c., | % RSD = 1.9 |
| | | 100.0% l.c., | Acceptance Value (AV) |
| | | 99.8% l.c.) | Limit: 5.0 |
| Microbiological | | | |
| Testing | | | |
| Total Aerobic | NMT 1000 cfu/g | NT | LT 10 cfu/g |
| Microbial Count | | | |
| Total Combined | NMT 100 cfu/g | NT | LT 10 cfu/g |
| Yeast and Mold | | | |
| Count | | | |
| Escherichia coli | Absence in 10 g | NT | Absence in 10 g |

NT = Not Tested
* ND = Not Detected; LOD = 0.03%; LOQ = 0.1%; NQ = Not Quantifiable (≧0.03% but ≦0.1%); these LOD and LOQ levels were used to report impurities for the development Lot 10MC-45.
** ND = Not Detected; LOD = 0.02%; LOQ = 0.05%
l.c. = label claim
LT = Less than

### EXAMPLE 9

### Clinical Trials

### Phase I Clinical Trials, Relative Bioavailability and Food Effect Study of S-I

### Protocol Summary

### Title: Phase I, Relative Bioavailability and Food Effect Study of S-I

Primary Objectives: To assess the relative bioavailability of a softgel formulation of S-I with a formulated dry powder capsule that has previously been used in the early phase clinical development program
To assess the effect of food on the pharmacokinetics of S-I softgel, 3 mg
   Secondary Objective: To assess the safety and tolerability of S-I.
   Design: This is a single center, randomized, open label, three period, crossover relative bioavailability and food effect study.
   Planned Number of Subjects: A total of 27 subjects were enrolled into the trial.
   Treatment duration: Eligible subjects received three single doses of S-I 3 mg over a 15 day period where doses were administered on Day 1, Day 8, and Day 15
   Treatments: S-I softgel, 3 mg, without food
S-I Capsule, 3 mg, without food
S-I softgel, 3 mg, with food

### Introduction

### Background, Target Indication and Pharmacologic Activity

The prevalence of cachexia increases from 50 percent at presentation to more than 80 percent before death from malignancy. In over 20 percent of cancer patients, cachexia is the cause of death (Bruera E. Anorexia, cachexia and nutrition. Br. Med. J. 1997; 315: 1219-1222). Cancer cachexia leads to shorter survival, decreased response rates and increased toxicity to chemotherapy, weakness, and an overall decreased quality of life (DeWys et al. Prognostic effect of weight loss prior to chemotherapy in cancer patients. Eastern Cooperative Oncology Group. Am J Med. 1980 October; 69(4): 491-497).

A Phase II double-blind, placebo-controlled study was performed with patients with cancer that had experienced at least a 2% total body weight loss in the 6 months prior to the study. The subjects received S-I or matching placebo for 4 months. In this study, no exercise program was mandated and no diet control or monitoring was conducted. The primary endpoint of this study was total lean body mass. The key secondary endpoint of this study was physical function (stair climb speed and power). The findings of this study at the 3 mg dose are summarized below:
a statistically significant increase in total lean body mass was observed (p=0.015 compared to placebo, p=0.020 compared to baseline, by central radiology read),
a statistically significant decrease in the time it took the subjects to climb 12 steps was observed (p=0.007 compared to baseline, p=0.028 compared to placebo), and
a statistically significant increase in the power subjects were able to exert climbing 12 steps was also observed (p=0.001 compared to baseline, p=0.058 compared to placebo).

During the course of this study, 437.5 mL of blood was taken from each subject.

### Study Objectives

### Primary Objectives

To assess the relative bioavailability of a softgel formulation of S-I compared to a formulated dry powder filled capsule (Capsule).

To assess the effect of food on the pharmacokinetics of S-I softgel, 3 mg

### Secondary Objectives

To assess the safety and tolerability of S-I.

### Study Design

This was a single center, randomized, open label, three period, crossover relative bioavailability and food effect study. The eligible subjects (healthy young males from 19 to 45 years of age) received either S-I softgel, 3 mg, without food, S-I Capsule, 3 mg, without food, or S-I softgel, 3 mg, with food in a three period crossover design.

### Endpoints

To compare the AUC_{0-T} of the S-I softgel, 3 mg, without food with the S-I Capsule, 3 mg, without food.

To compare the AUC_{0-T} of the S-I softgel, 3 mg, without food with the S-I softgel, 3 mg, with food

### Treatment Groups and Allocation of Subjects

A total of 27 subjects (healthy males aged 19-45 years, having a bone mass index (BMI) between 18 and 32) were enrolled into this study. The subjects were randomized into one of three groups. The treatment groups and allocation of dosing within the periods is provided in the table below.

TABLE 15
Treatment groups and allocation of subjects.

| | Period 1 | Period 2 | Period 3 |
|---|---|---|---|
| Group 1 (n = 9) | Treatment A | Treatment B | Treatment C |
| Group 2 (n = 9) | Treatment B | Treatment C | Treatment A |
| Group 3 (n = 9) | Treatment C | Treatment A | Treatment B |

Treatment A = S-I softgel, 3 mg, without food
Treatment B = S-I Capsule, 3 mg, without food
Treatment C = S-I softgel, 3 mg, with food

### Study Duration

Each subject randomized into this study received three doses of S-I. The doses were administered on Day 1, Day 8, and Day 15. A follow up visit was conducted on Day 21. All 27 subjects were dosed on the same days. The total duration of the study from date of first dose to follow up visit was 21 days.

### Study Medication

### Randomizations and Blinding

The study was a randomized, open label study with 27 subjects randomized to one of three groups (9 subjects/group).

### Pharmacokinetic Samples

### Blood Sampling Times

Samples of venous blood for the determination of plasma S-I and related metabolite concentrations were obtained at 0 (just prior to dose), 0.5, 1, 2, 3, 4, 6, 8, 12, 16, 24, 36, 48, 72, 96, and 120 hours post-dose on Days 1, 8, and 15.

### Urine Sample Collection

Samples of urine were collected for 24 hours after the first dose on Day 1 for the qualitative and/or quantitative analysis of S-I and related metabolites.

### Statistical and Analytical Methods

### Calculation of Pharmacokinetic (PK) Variables

Pharmacokinetic variables were calculated from the plasma concentration data using standard, noncompartmental methods. The definition and method of determination for each pharmacokinetic variable is summarized in the following table.

TABLE 16
Pharmacokinetic Variables

| Variable | Definition |
|---|---|
| Cₘₐₓ | Maximum plasma concentration; the highest |
| | concentration observed during a dosage interval |
| Tₘₐₓ | The time that Cₘₐₓ was observed |
| Cₜ | The last measured plasma concentration; the last |
| | concentration above the lower LOQ (Limit of |
| | Quantitation) following a dose |
| λ_{z} (Ke) | The terminal elimination rate constant; |
| | calculated using linear regression on the |
| | terminal portion of the Ln-concentration versus |
| | time curve |
| T½ | Terminal elimination half-life; calculated as |
| | 0.693/λ_{z} |
| AUC_{T} | Area under the concentration versus time curve |
| | from time 0 to the last measured concentration |
| | (Cₜ); calculated using linear trapezoid rule |
| AUC₀₋₂₄ | Area under the concentration versus time curve |
| | from time 0 to the 24 hour measured concentration; |
| | calculated using the linear trapezoidal rule |
| AUC | Area under the concentration versus time curve |
| | from time 0 to infinity; calculated as AUC_{T} + |
| | Cₜ/λ_{z} |

### Assessment of Relative Bioavailability

Comparison of mean AUC₀₋₂₄, AUC_{T} and Cₘₐₓ was conducted between S-I Capsule, 3 mg and S-I softgel, 3 mg.

The standard 90 percent confidence interval (CI) for the ratio of population geometric means between the each capsule formulation and oral solution, based on log-transformed data, is contained in the bioequivalence (BE) limits of 80-125 percent for AUC and Cₘₐₓ was used to establish relative bioavailability of S-I Capsule, 3 mg and S-I softgel, 3 mg.

The Tₘₐₓ values were compared observationally for similarity between S-I Capsule, 3 mg and S-I softgel, 3 mg.

### Assessment of the Effect of Food

Comparison of mean AUC₀₋₂₄, AUC_{T} and Cₘₐₓ were conducted between the fasting and fed data from the S-I softgel, 3 mg.

The standard 90 percent CI for the ratio of population geometric means between the fasting and fed data within each sequence, based on log-transformed data, is contained in the BE limits of 80-125 percent for AUC and Cₘₐₓ was used to establish the effect of food on the bioavailability of S-I softgel, 3 mg.

The Tₘₐₓ values were compared observationally for similarity between the fasting and fed data within each sequence.

### Summary of PK Results

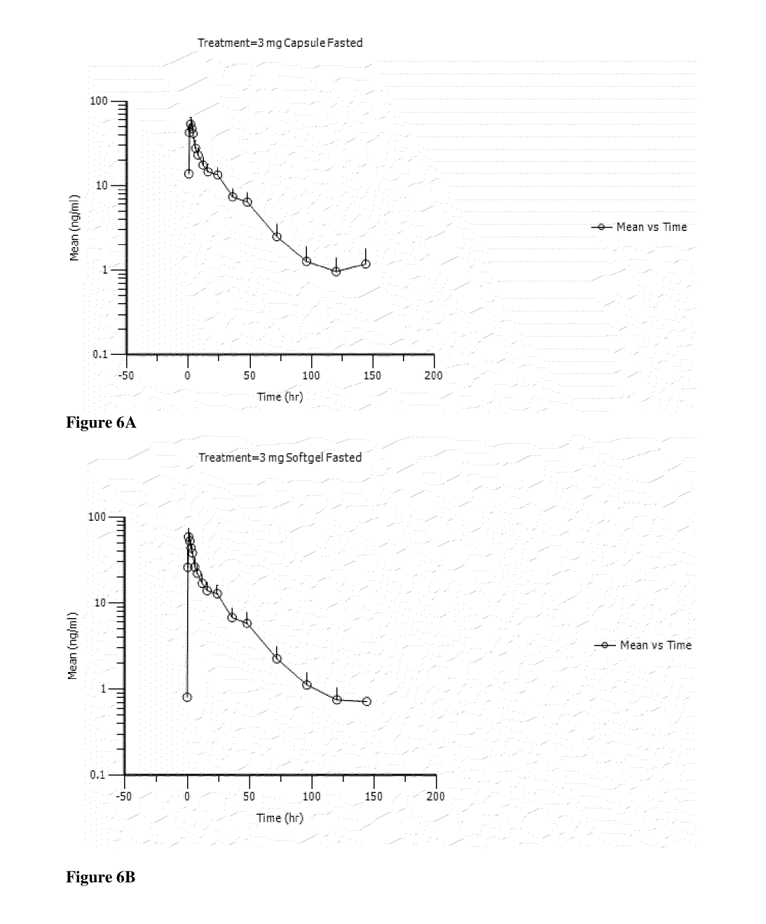

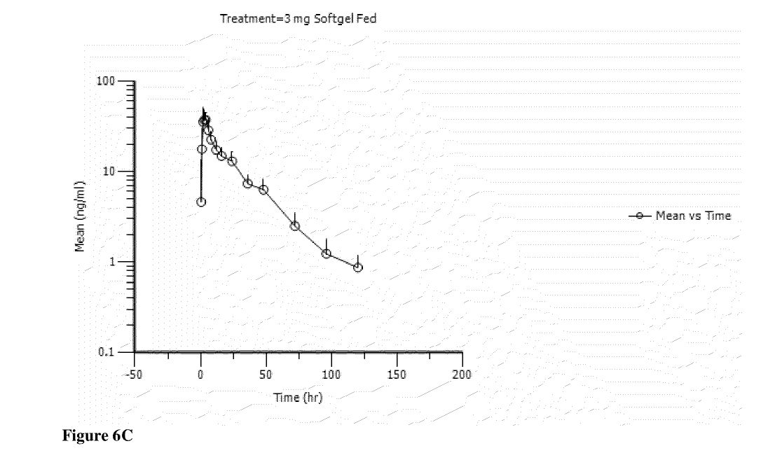

Summary of the PK data from the study is presented in FIG. 6 and Table 17 below.

Tₘₐₓ, Cₘₐₓ were calculated for each dosage form taken by each patient. AUCₐₗₗ was calculated for each dosage form taken by each patient using the linear trapezoidal method incorporating all time points with measureable plasma concentrations. Geometric means and 90% confidence intervals for the Cₘₐₓ and AUCₐₗₗ parameters were calculated whereas the arithmetic mean and standard deviation were used to describe the Tₘₐₓ parameter. The similar means for AUCₐₗₗ with broadly overlapping confidence intervals suggest the extent of absorption was not different between dosage forms. This finding was supported by the determination of equivalence according to FDA guidelines (Balthasar J. P., Bioequivalence and Bioequivalency Testing, American Journal of Pharmaceutical Education, Vol. 63, 1999, 194-198). Though the 90% confidence intervals for the Cₘₐₓ parameter do not overlap between dosage forms, they too were determined equivalent using FDA methodology. The similarity in AUCₐₗₗ parameters between fed and fasted states in patients administered the softgel formulation suggests the food effect was not relevant.

TABLE 17
Summary of the PK data from the Phase I clinical study

| | | | | | CI 90% | CI 90% |
|---|---|---|---|---|---|---|
| | | N | Mean | SD | Lower | Upper |
| | | 3 mg Capsule Fasted | | | | |
| AUCₐₗₗ | hr*ng/mL | 26 | 885.884 | — | 823.499 | 952.996 |
| Cₘₐₓ | ng/mL | 26 | 53.46 | — | 50.117 | 57.025 |
| Tₘₐₓ | hr | 26 | 2.04 | 0.45 | — | — |
| | | 3 mg Softgel Fasted | | | | |
| AUCₐₗₗ | hr*ng/mL | 25 | 847.059 | — | 783.314 | 915.992 |
| Cₘₐₓ | ng/mL | 25 | 60.827 | — | 57.037 | 64.868 |
| Tₘₐₓ | hr | 25 | 1.27 | 0.53 | — | — |
| | | 3 mg Softgel Fed | | | | |
| AUCₐₗₗ | hr*ng/mL | 26 | 822.191 | — | 761.48 | 887.75 |
| Cₘₐₓ | ng/mL | 26 | 41.703 | — | 38.802 | 44.82 |
| Tₘₐₓ | hr | 26 | 2.885 | 1.107 | — | — |

### EXAMPLE 10

### Delivery System of S-I

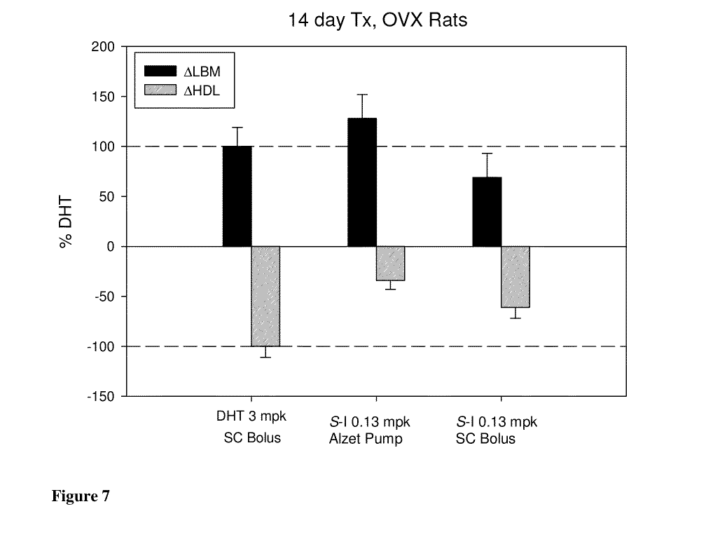

Female rats were ovariectomized and randomized by body weight into groups of 10. Treatment groups included: (1) 3 mg/kg/day DHT administered daily via subcutaneous injection, (2) 0.13 mg/kg/day S-I administered daily via subcutaneous injection, and (3) 0.13 mg/kg/day S-I administered at a constant rate via subcutaneous infusion by an Alzet pump. Baseline measurements of fasting serum HDL as determined by enzymatic assay were performed via retroorbital blood draw for all animals. Baseline lean body mass (LBM) as determined by magnetic resonance was determined for all animals. After 14 days of treatment, changes from baseline in lean body mass (Δ LBM) and fasting serum HDL (Δ HDL) were determined and represented as a percentage of DHT treated animals. Unexpectedly, as can be shown in FIG. 7, the administration of S-I by constant rate subcutaneous infusion caused a larger increase in LBM than daily subcutaneous injections, but was associated with a smaller decrease in serum HDL, suggesting sustained subcutaneous delivery of the drug (e.g., topical, transdermal, or infusion) may maximize efficacy and lessen effects on serum lipids.

The exposure of S-I in female rats was AUCinf of 147,000 ng*hr/ml following a single 10 mg per kg IV dose. With the reasonable assumption that the SC Alzet pump dose was completely absorbed, a 0.13 mg per kg dose should result in an exposure of −1911 ng*hr/mL in rats. This exposure corresponds to the steady-state AUC(0-t) of 1752 ng*hr/mL resulting from 10 mg daily doses in healthy young men (G100402, MAD). In other words, the 0.13 mg per kg per day Alzet dose in rats is equivalent to a 10 mg per day continuous release dose in humans, such as might be accomplished by a mechanical or osmotic pump, or transdermal patch, or the like.

### EXAMPLE 11

### Pharmaceutical Compositions

### Softgel Capsules Comprising Compound S-I, 0.5 mg

S-I was developed as a formulated soft gelatin capsule (softgel) with a dosage strength of 0.5 mg. S-I 0.5 mg softgel drug product was provided as an opaque, off-white oval softgel with “GTx” printed in black on the outer shell.

### Quantitative Composition

The quantitative composition of S-I 0.5 mg softgels is described in Table 18.

TABLE 18
Batch Formula S-I 3.0 mg Softgels

| | Unit Formula |
|---|---|
| Component | (mg/softgel) |
| S-I | 0.5 |
| Polyethylene Glycol 400, NF, EP | 299.5 |
| Total Weight | 300.0 |

### Batch Analysis (S-I 0.5 mg Softgels)

Batch analysis data for S-I 0.5 mg softgels are provided in Table 19. The batch was manufactured according to the details described in the section of Example 8 titled “Description of Manufacturing Process and Process Controls” presented above, and tested by the methods described in the section of Example 8 titled “Analytical Procedures” presented above.

TABLE 19
Batch Analysis Data for S-I 0.5 mg Softgels

| Usage | | Development, Stability | | | | |
|---|---|---|---|---|---|---|
| Test | Specification | Batch Results | | | | |
| Identification | Retention time of the major peak | Retention time of the major peak | | | | |
| by HPLC | obtained from sample solution | obtained from sample solution | | | | |
| Retention time | corresponds that of S-I reference | corresponds that of S-I reference | | | | |
| | standard ± 0.25 minutes | standard. Retention time difference: | | | | |
| | | 0.00 minutes | | | | |
| Identification | UV Spectra (200 nm to 400 nm) of | UV Spectra(200 nm to 400 nm) of | | | | |
| by UV | the major peak in the sample | the major peak in the sample | | | | |
| | preparation compares to that of | preparation compares to that of | | | | |
| | the standard preparation using a | the standard preparation using a | | | | |
| | HPLC diode array UV detector | HPLC diode array UV detector. | | | | |
| Assay (HPLC) | 95.0-105.0% of label claim | Sample | % LC | | | |
| | | Prep. 1 | 98% | | | |
| | | Prep. 2 | 98% | | | |
| | | Average | 98% | | | |
| | | (n = 2) | | | | |
| Disintegration | | Sample | Minutes | | | |
| (minutes) | | 1 | 7 | | | |
| | | 2 | 7 | | | |
| | | 3 | 7 | | | |
| | | 4 | 7 | | | |
| | | 5 | 7 | | | |
| | | 6 | 7 | | | |
| | | Max | 7 | | | |
| | | time | | | | |
| Hardness (N) | | Sample | Newtons | | | |
| | | 1 | 9.0 | | | |
| | | 2 | 9.0 | | | |
| | | 3 | 8.9 | | | |
| | | 4 | 8.7 | | | |
| | | 5 | 8.8 | | | |
| | | Average | 8.9 | | | |
| Moisture | | Sample | % water | | | |
| (% w/w) | | 1 | 8.5% | | | |
| | | 2 | 7.6% | | | |
| | | Average | 8.1% | | | |
| | | (n = 2) | | | | |
| Content | Meets USP <905> | 98.8, 98.9, 98.8, 98.7, 98.7, 98.6, | | | | |
| Uniformity | Requirements | 97.3, 97.6, 96.9, 96.8. | | | | |
| | | Average = 98.1% LC; | | | | |
| | | % RSD = 0.8; | | | | |
| | | Acceptance Value (AV) Limit: 2.4 | | | | |
| Dissolution | Report results at 10, 20, 30 | | % Dissolved | | | |
| | and 60 minutes | | 10 | 20 | 30 | 60 |
| | | Sample | min | min | min | min |
| | | 1 | 95 | 97 | 98 | 98 |
| | | 2 | 52 | 94 | 96 | 96 |
| | | 3 | 96 | 96 | 95 | 96 |
| | | 4 | 97 | 97 | 96 | 96 |
| | | 5 | 96 | 97 | 96 | 97 |
| | | 6 | 96 | 97 | 97 | 97 |
| | | Avg. | 89 | 96 | 96 | 97 |
| | | % RSD | 20.4 | 1.2 | 0.9 | 0.7 |
| Microbiological | | | | | | |
| Testing | | | | | | |

| | | |
|---|---|---|
| Total Aerobic | NMT 1000 cfu/g | LT 10 cfu/g |
| Microbial Count | | |
| Total Combined | NMT 100 cfu/g | LT 10 cfu/g |
| Yeast and Mold | | |
| Count | | |
| Escherichia coli | Absence in 10 g | Absent in 10 g |

NT = Not Tested
* ND = Not Detected; LOD = 0.02%; LOQ = 0.05%;
LC = label claim
LT = Less than

It will be appreciated by a person skilled in the art that the present invention is not limited by what has been particularly shown and described hereinabove. Rather, the scope of the invention is defined by the claims that follow:

What is claimed is:

1. A pharmaceutical composition comprising a softgel capsule comprising the S-isomer of Compound I: 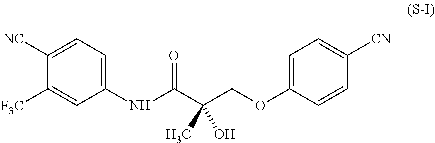 [SMILES: C[C@](O)(COc1ccc(C#N)cc1)C(=O)Nc1ccc(C#N)c(C(F)(F)F)c1]
or its isomer, polymorph, pharmaceutically acceptable salt, N-oxide, hydrate or any combination thereof, and a carrier, diluent, or a combination thereof.

2. The pharmaceutical composition of claim 1, wherein said carrier or diluent is a polyethylene glycol, or mixtures thereof.

3. The pharmaceutical composition of claim 1, further comprising a solubilizer or filler.

4. The pharmaceutical composition of claim 3, wherein said solubilizer or filler is polyethylene glycol.

5. The pharmaceutical composition of claims 1, further comprising one or more additives selected from a capsule shell polymer, placticizer, colorant or opacifier, binder, a disintegrant, a buffer, a surfactant, an emulsifier, a stabilizing agent, a viscosity increasing agent, a sweetener, a film forming agent, or any combination thereof.

6. The pharmaceutical composition of claim 5, wherein said capsule shell polymer is gelatin.

7. The pharmaceutical composition of claim 5, wherein said plasticizer is sorbitol.

8. The pharmaceutical composition of claim 7, wherein said sorbitol is sorbitol special—glycerin blend.

9. The pharmaceutical composition of claim 5, wherein said colorant or opacifier is titanium dioxide.

10. The pharmaceutical composition of claim 1, wherein said softgel capsule comprises Compound S-I, polyethylene glycol, gelatin, sorbitol, and titanium dioxide.

11. The pharmaceutical composition of claim 1, wherein said softgel capsule consists essentially of 3 mg of Compound S-I.

12. The pharmaceutical composition of claim 1, wherein said softgel capsule consists essentially of 1 mg of Compound S-I.

13. The pharmaceutical composition of claim 1, wherein said softgel capsule consists essentially of 0.5 mg of Compound S-I.

14. A pharmaceutical composition comprising a micronized S-isomer of Compound I: 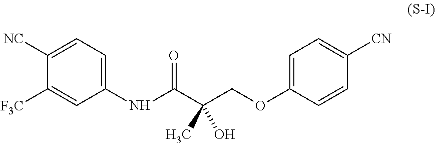 [SMILES: C[C@](O)(COc1ccc(C#N)cc1)C(=O)Nc1ccc(C#N)c(C(F)(F)F)c1]
or its isomer, polymorph, pharmaceutically acceptable salt, N-oxide, hydrate or any combination thereof, and a carrier, diluent, or a combination thereof.

15. The pharmaceutical composition of claim 14, having a micronized mean particle size of between about 0.5-200 μm, or its isomer, polymorph, pharmaceutically acceptable salt, N-oxide, hydrate or any combination, and a carrier, diluent, or any combination thereof.

16. The pharmaceutical composition of claim 14 wherein said carrier or diluent is a pregelatinized starch, lactose monohydrate, a cellulosic material, or mixtures thereof.

17. The pharmaceutical composition of claim 16, wherein said cellulosic material is microcrystalline cellulose.

18. The pharmaceutical composition of claim 14, further comprising a lubricant.

19. The pharmaceutical composition of claim 18, wherein said lubricant is magnesium stearate.

20. The pharmaceutical composition of claim 14, further comprising a glidant.

21. The pharmaceutical composition of claim 20, wherein said glidant is colloidal silicon oxide.

22. The pharmaceutical composition of claims 14, further comprising one or more additives selected from a binder, a disintegrant, a buffer, a surfactant, a solubilizing agent, a plasticizer, an emulsifier, a stabilizing agent, a viscosity increasing agent, a sweetener, a film forming agent, or any combination thereof.

23. The pharmaceutical composition of claim 22, wherein said surfactant is sodium lauryl sulfate.

24. The pharmaceutical composition of claim 14, wherein said composition is in a solid form.

25. The pharmaceutical composition of claim 24, wherein said composition is in the form of a capsule.

26. The pharmaceutical composition of claim 25, wherein said capsule comprises Compound S-I, pregelatinized starch, lactose monohydrate, microcrystalline cellulose, sodium lauryl sulfate, colloidal silicon dioxide and magnesium stearate.

27. The pharmaceutical composition of claim 25, wherein said capsule comprises 0.5 mg of Compound S-I.

28. The pharmaceutical composition of claim 25, wherein said capsule comprises 1 mg of Compound S-I.

29. The pharmaceutical composition of claim 25, wherein said capsule comprises 3 mg of Compound S-I.

30. The pharmaceutical composition according of claim 24, wherein said composition is in the form of a tablet.

31. The pharmaceutical composition of claim 30, wherein said tablet comprises Compound S-I, pregelatinized starch, lactose monohydrate, microcrystalline cellulose, sodium lauryl sulfate, colloidal silicon dioxide and magnesium stearate.

32. The pharmaceutical composition of claim 30, wherein said tablet comprises 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.

33. The pharmaceutical composition of claim 32, wherein said tablet comprises 0.1 mg of Compound S-I.

34. A sustained release pharmaceutical composition comprising an S-isomer of Compound I: 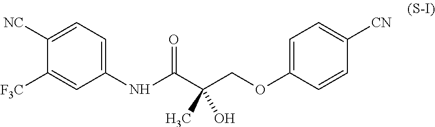 [SMILES: C[C@](O)(COc1ccc(C#N)cc1)C(=O)Nc1ccc(C#N)c(C(F)(F)F)c1]
or its isomer, polymorph, pharmaceutically acceptable salt, N-oxide, hydrate or any combination thereof, and a carrier, diluent, or a combination thereof.

35. The sustained release pharmaceutical composition of claim 34, wherein said composition is administered topically, transdermally, orally or by infusion.

36. The sustained release pharmaceutical composition of claim 35, wherein said topical administration is via an ointment, a cream, an oil or any combination thereof.

37. The sustained release pharmaceutical composition of claim 35, wherein said transdermal administration is via a transdermal patch.

38. The sustained release pharmaceutical composition of claim 35, wherein said oral administration is via a controlled release oral dosage form.

39. The sustained release pharmaceutical composition of claim 35, wherein said infusion is via an osmotic pump or mechanized pump.

40. The pharmaceutical composition of claim 34, wherein said composition comprises 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.

41. The pharmaceutical composition of claim 34, wherein said composition comprises a daily delivery rate of 0.1 mg, 0.3 mg, 0.5 mg, 1 mg, 1.5 mg, 2 mg, 2.5 mg, 3 mg, 4 mg, 5 mg, 9 mg, 10 mg or 30 mg of Compound S-I.
